(19) United States
(12) Patent Application Publication
(10) Pub. No.: US 2010/0172874 A1
(43) Pub. Date: Jul. 8, 2010

(54) GUT MICROBIOME AS A BIOMARKER AND THERAPEUTIC TARGET FOR TREATING OBESITY OR AN OBESITY RELATED DISORDER

(75) Inventors: Peter J. Turnbaugh, St. Louis, MO (US); Ruth E. Ley, St. Louis, MO (US); Michael A. Mahowald, St. Louis, MO (US); Jeffrey I. Gordon, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: THE WASHINGTON UNIVERSITY, St. Louis, MO (US)

(21) Appl. No.: 12/519,958

(22) PCT Filed: Dec. 10, 2007
(86) PCT No.: PCT/US2007/087003

§ 371 Date: Nov. 12, 2009

Related U.S. Application Data
(60) Provisional application No. 60/870,533, filed on Dec. 18, 2006.
(60) Provisional application No. 60/870,785, filed on Dec. 19, 2006.
(60) Provisional application No. 60/871,049, filed on Dec. 20, 2006.

(51) Int. Cl. A61K 35/74; A61P 3/04; C12Q 1/02; C40B 30/06; C40B 30/02

(52) U.S. Cl. 424/93.4; 435/29; 506/10; 506/8

(57) ABSTRACT

The present invention relates to the gut microbiome as a biomarker and therapeutic target for energy harvesting, weight loss or gain, and/or obesity in a subject. In particular, the invention provides methods of altering and monitoring the relative abundance of Bacteroides and Firmicutes in the gut microbiome of a subject.

### FIELD OF THE INVENTION

The present invention relates to the gut microbiome as a biomarker and therapeutic target for energy harvesting, weight loss or gain, and/or obesity in a subject.

### BACKGROUND OF THE INVENTION

According to the Center for Disease Control (CDC), over sixty percent of the United States population is overweight, and greater than thirty percent are obese. This translates into more than 50 million adults in the United States with a Body Mass Index (BMI) of 30 or above. Obesity is also a worldwide health problem with an estimated 500 million overweight adult humans [body mass index (BMI) of 25.0-29.9 kg/m²] and 250 million obese adults (Bouchard, C (2000) N Engl J. Med. 343, 1888-9). This epidemic of obesity is leading to worldwide increases in the prevalence of obesity-related disorders, such as diabetes, hypertension, as well as cardiac pathology, and non-alcoholic fatty liver disease (NAFLD; Wanless, and Lentz (1990) Hepatology 12, 1106-1110. Silverman, et al, (1990). Am. J. Gastroenterol. 85, 1349-1355; Neuschwander-Tetri and, Caldwell (2003) Hepatology 37, 1202-1219). According to the National Institute of Diabetes, Digestive and Kidney Diseases (NIDDK) approximately 280,000 deaths annually are directly related to obesity. The NIDDK further estimated that the direct cost of healthcare in the U.S. associated with obesity is $51 billion. In addition, Americans spend $33 billion per year on weight loss products. In spite of this economic cost and consumer commitment, the prevalence of obesity continues to rise at alarming rates. From 1991 to 2000, obesity in the U.S. grew by 61%.

Although the physiologic mechanisms that support development of obesity are complex, the medical consensus is that the root cause relates to an excess intake of calories compared to caloric expenditure. While the treatment seems quite intuitive, dieting is not an adequate long-term solution for most people; about 90 to 95 percent of persons who lose weight subsequently regain it. Although surgical intervention has had some measured success, the various types of surgeries have relatively high rates of morbidity and mortality.

Pharmacotherapeutic principles are limited. In addition, because of undesirable side effects, the FDA has had to recall several obesity drugs from the market. Those that are approved also have side effects. Currently, two FDA-approved anti-obesity drugs are orlistat, a lipase inhibitor, and sibutramine, a serotonin reuptake inhibitor. Orlistat acts by blocking the absorption of fat into the body. An unpleasant side effect with orlistat, however, is the passage of undigested oily fat from the body. Sibutramine is an appetite suppressant that acts by altering brain levels of serotonin. In the process, it also causes elevation of blood pressure and an increase in heart rate. Other appetite suppressants, such as amphetamine derivatives, are highly addictive and have the potential for abuse. Moreover, different subjects respond differently and unpredictably to weight-loss medications.

Because surgical and pharmacotherapy treatments are problematic, new non-cognitive strategies are needed to prevent and treat obesity and obesity-related disorders.

### BRIEF DESCRIPTION OF THE FIGURES

FIG. 1 depicts a graph showing the effect of decreasing e-value cut-offs on ECT assignments to the KEGG database from pyrosequencer and capillary sequencer datasets. Points indicate the average number of KO assignments per kb of microbiome sequence. Mean values±s.e.m. are plotted. The GS20 pyrosequencer and the 3730xl capillary sequencer both resulted in an average 0.3 KO (KEGG orthology) assignments per kb of sequence at an e-value cutoff <10⁻⁵. However, the number of EGTs present in the pyrosequencer-derived datasets rapidly decays as the e-value cutoff is decreased, whereas the number of EGTs present in the capillary sequencer datasets is relatively stable to <10⁻³.

FIG. 2 depicts a graph and tables showing the comparison of datasets obtained from the cecal microbiomes of obese and lean littermates. (A) Number of observed orthologous groups in each cecal microbiome. Black indicates the number of observed groups. Grey indicates the number of predicted missed groups. (B) Relative abundance of a subset of COG categories (BLASTX, e-value <10⁻⁵) in the lean1 (black) and ob1 (white) cecal microbiome, characterized by capillary- and pyro-sequencers (square, and triangles, respectively). A subset of COG categories (C) and all KEGG pathways (D) consistently enriched or depleted in the cecal microbiomes of both obese mice compared to their lean littermates. Red denotes enrichment and green indicates depletion based on a cumulative binomial test (brightness indicates level of significance). Black indicates pathways whose representation is not significantly different. Asterisks indicate groups that were consistently enriched or depleted between both sibling pairs using a more stringent EGT assignment strategy (e-value<10⁻⁸).

FIG. 3 depicts graphs showing the taxonomic assignments of EGTs and 16S rRNA gene fragments. Relative abundance of EGTs (reads assigned to NR, BLASTX with an e-value<10⁻⁵) in each cecal microbiome confirms the presence of the indicated bacterial divisions in addition to Euryarcheota. Metazoan sequences (including Mus musculus and fungi) are also present at low abundance. Bacterial divisions with greater than 1% representation in at least three microbiomes are shown. (B) Alignment of 16S rRNA gene fragments (black) confirms our previous PCR-derived 16S rRNA gene sequence-based survey (white). Comparisons include all microbiomes sampled with the capillary sequencer (square) and the two microbiomes sampled with the pyrosequencer (triangle).

FIG. 4 depicts a graph showing that microbiomes cluster according to host genotype. (A) Clustering of cecal microbiomes of obese and lean sibling pairs based on reciprocal TBLASTX comparisons. All possible reciprocal TBLASTX comparisons of microbiomes (defined by capillary sequencing) were performed from both lean and obese sibling pairs. A distance matrix was then created using the cumulative bitscore for each comparison and the cumulative score for each self-self comparison. Microbiomes were subsequently clustered using NEIGHBOR (PHYLIP version 3.64). (B) Principal Component Analysis (PCA) of KEGG pathway assignments. A matrix was constructed containing the number of EGTs assigned to each KEGG pathway in each microbiome (includes KEGG pathways with >0.6% relative abundance in at least two microbiomes, and a standard deviation >0.3 across all microbiomes), PCA was performed using Cluster3.0, and the results graphed along the first two components.

FIG. 5 depicts KEGG pathways that are enriched or depleted in the cecal microbiomes of both obese versus lean sibling pairs, as indicated by bootstrap analysis of relative gene content. Pathways that are consistently enriched or depleted in the pyrosequencer-based comparison of ob1 versus lean1 littermates, and the capillary sequencer-based comparison of ob2 versus leant littermates are shown. Red indicates enrichment and green indicates depletion (brightness denotes level of significance). Black indicates groups that are not significantly changed.

FIG. 6 depicts graphs showing the biochemical analysis and microbiota transplantation experiments confirm that the ob/ob microbiome has an increased capacity for dietary energy harvest (A) Gas chromatography-mass spectrometry quantification of SCFAs in the ceca of lean (black; +/+, ob/+; n=4) and obese (white; ob/ob; n=5) conventionally-raised C57BL/6J mice. (B) Bomb calorimetry of the fecal gross energy content (kcal/g) of lean (black; +/+, ob/+; n=9) and obese (white; ob/ob; n=13) conventionally-raised C57BL/6J mice. (C) Colonization of germ-free wild-type C57BL/6J mice with a cecal microbiota harvested from obese donors (white; ob/ob; n=9 recipients) results in a significantly greater percentage increase in total body fat than colonization with a microbiota from lean donors (black; +/+; n=10 recipients). Total body fat content was measured before and after a two-week colonization, using dual-energy x-ray absorptiometry. Mean values±s.e.m. are plotted. Asterisks indicate significant differences (two-tailed Student's t-Test of all datapoints, *p<0.05, **p<0.01, ***p<0.001).

FIG. 7 depicts analyses of microbial communities harvested from obese (ob/ob) and lean (+/+) C57BL/6J donor mice and colonized gnotobiotic recipients. Online Unifrac clustering of microbial community structure, based on 4,157 16S rRNA gene sequences (see Table 7 for number of sequences per sample; ARB tree available at http://gordonlab.wustl.edu/supplemental/Turnbaugh/obob/). Nodes denoted by a black square are robust to sequence number (jackknife values >0.70, representing the number of times the node was present when 166 sequences were randomly chosen for each mouse for n=100 replicates). Pie charts indicate the average relative abundance of Firmicutes (black), Bacteroidetes (white), and other (grey; includes Verrucomicrobia, Proteobacteria, Actinobacteria, TM7, and Cyanobacteria) in the donor and recipient microbial communities.

FIG. 8 depicts a graph of the relative abundance of COG categories (percentage of total EGTs assigned to COG using BLASTX and e-value<10⁻⁵) in the lean1 (black square), ob1 (white square), lean2 (black triangle), and ob2 (white triangle) cecal microbiomes. Microbiomes were characterized by capillary sequencing.

FIG. 9 depicts COGs that are enriched or depleted in the cecal microbiornes of both obese versus lean sibling pairs, as indicated by binomial comparisons of relative gene content. The COGs shown are enriched or depleted in the pyrosequencer-based comparison of ob1 versus lean1 littermates and the capillary sequencer-based comparison of ob2 versus lean2 littermates. Red indicates enrichment and green indicates depletion (brightness denotes level of significance). Black indicates groups that are not significantly changed.

FIG. 10 depicts the correlation between weight loss and gut microbial ecology. Clustering of 16S rRNA gene sequence libraries of fecal microbiota for each subject (color) and time point (T0=baseline, T1=12 weeks, T2=26 weeks, T3=52 weeks of diet therapy) in the two treatment groups, based on UniFrac analysis of the 18,348-sequence phylogenetic tree. (B) Relative abundance of the Bacteroidetes and Firmicutes. For each time point, the values from all available samples were averaged (n=11 or 12 per time point). Lean controls include 4 stool samples from two subjects taken 1 year apart, plus 3 stool samples published. Mean values±SE are plotted. (C) Change in Bacteroidetes relative abundance and weight loss above a threshold of 6% for the CARB-R diet and 2% for the FAT-R diet.

FIG. 11 depicts an illustration of the experimental design. (A) Diet-induced obesity (DIO) in germ-free mice colonized with a complex microbial community. (B) Conventionally-raised (CONV-R) wild-type mice fed a Western or CHO diet. (C) Specific dietary shifts after two months on the Western diet. (D) Microbiota transplantation experiments from donor mice on multiple diets to lean germ-free CHO-fed recipients. Numbers in parentheses refer to the age of mice at each step in the protocol. Mouse diets are labeled Western, FAT-R, CARB-R, and CHO (see Tables 11 and 12).

FIG. 12 depicts data showing that diet-induced obesity alters gut microbial ecology in conventionalized mice. Adult C57BL/6J conventionalized mice were fed a low-fat high-polysaccharide (CHO) or high-fat/high-sugar (Western) diet. 16S rRNA gene sequence-based surveys were performed on the distal gut (cecal) contents of ten mice (n=5 mice/group) and the cecal contents from the donor mouse. UniFrac-based analysis of community membership (who's there) indicates that the communities cluster based on diet: the community from CHO fed recipients clusters with the CHO fed donor cecal microbiota, whereas the community from Western diet fed recipients has been altered. Black boxes indicate nodes that were reproduced in >70% of all jackknife replications (n=96 sequences). The relative abundance of the Firmicutes is increased in the Western diet microbiota, corresponding to a bloom in the Mollicutes class. Pie charts show the average relative abundance of bacterial lineages in the CHO diet versus Western diet cecal microbiota (n=5 mice/group). The asterisk indicates that the sample was also analyzed based on whole community shotgun sequencing.

FIG. 13 depicts graphs showing that diet-induced obesity (DIO) is linked to changes in gut microbial ecology, resulting in an increased capacity of the distal gut microbiota to promote host adiposity. (A) The relative abundance (% of total 16S rRNA gene sequences) of the Firmicutes and Bacteroidetes divisions in the distal gut (cecal) microbiota of conventionalized, wild-type C57BL/6J mice fed a standard low-fat high-polysaccharide chow diet (CHO; n=5) or a high-fat/high-sugar Western diet (n=5). (B) DIO is associated with a marked reduction in the overall diversity of the cecal bacterial community. The Shannon index of diversity was calculated at multiple phylotype cutoffs (defined by % identity of 16S rRNA gene sequences) for each individual cecal dataset using DOTUR [13]. The average diversity at each cutoff is plotted for mice fed the CHO and Western diets. (C) DIO is linked to a bloom of the Mollicutes class of bacteria within the Firmicutes division. The relative abundance of the Mollicutes is shown for conventionalized mice fed the CHO or Western diet. (D) Microbiota transplantation experiments reveal that the DIO community has an increased capacity to promote host fat deposition. Total body fat was measured using dual-energy x-ray absorptiometry (DEXA) before and after a two-week colonization of adult germ-free CHO-fed C57BL/6J wild-type mice with a cecal microbiota harvested from mice maintained on CHO or Western diet (n=14 mice/treatment group). Mean values±SEM are shown. Asterisks in panels A-D indicate that the differences are statistically significant (Student's t-test, p<0.05), after using the Bonferroni correction to limit false positives.

FIG. 14 depicts the phylogeny of selected representatives from the Firmicutes division, including the Mollicute bloom and closely related human strains. 16S rRNA gene sequences for previously sequenced Firmicute genomes and Mollicute strains isolated from the human gut were identified in the RDP database [34]. All Mollicute sequences obtained from conventionalized C57BL/6J mice fed a CHO or Western diet (n=801 sequences) and from our previous survey of obese humans (length>1250 nucleotides; n=571 sequences) [9] were separately binned into phylotypes using DOTUR (99% identity) [13]. One representative of each of the six dominant mouse phylotypes was chosen (together comprising 81% of the mouse Mollicute sequences) in addition to one representative of each of the ten dominant human phylotypes. Likelihood parameters were determined using Modeltest [35] and a maximum-likelihood tree was generated using PAUP [36]. Bootstrap values represent nodes found in >70 of 100 repetitions. Phylotypes from the Mollicute bloom are shown in blue; wedge size is proportional to the indicated relative abundance (% of Mollicute 16S rRNA gene sequences). The Mollicute bloom and relatives are shaded in blue, previously sequenced Mollicutes (including the obligate parasites, Mycoplasma, and Mesoplasma florum) are shaded in yellow, and recently sequenced Firmicutes found in the normal distal human gut microbiota are shaded in red. Akkermansia muciniphila, a Verrucomicrobia, was used to root the tree (shaded in green).

FIG. 15 depicts a graph showing the Mollicute bloom occurs in conventionally-raised wild-type C57BL/6J mice as well as in mice without an intact innate or adaptive immune system. Wild-type (+/+), MyD88−/−, or Rag1−/− C57BL/6J mice were weaned onto a standard low-fat polysaccharide-rich (CHO) or high-fat/high-sugar (Western) diet. 16S rRNA gene sequence-based surveys were performed; sequences were aligned [41], and inserted into an ARB neighbor-joining tree [42]. Asterisks indicate significant differences (Student's t-test p<0.001).

FIG. 16 depicts a graph showing mice with diet-induced obesity that are switched to a FAT-R or CARBR diet exhibit stabilization of weight, decreased caloric intake and reduced adiposity. (A) Weight gain (g) and (B) percentage epidydymal fat-pad weight to body weight in wild type C57BL/6J mice that were initially weaned onto a Western diet for 8 weeks, and then maintained on the Western diet, or switched to a FAT-R or CARB-R diet for four weeks (n=5-6 mice/treatment group). Weight was monitored during the four week period. (C) Chow consumption (kcal/d) is decreased in mice switched to a FAT-R or CARB-R diet. Data are represented as mean±SEM. Asterisks indicate significant differences (ANOVA of FAT-R or CARB-R versus Western, *p<0.05, **p<0.01, ***p<0.0001).

FIG. 17 depicts data showing that switching from a Western to FAT-R or CARB-R diet results in a division-wide increase in the relative abundance of Bacteroidetes, and a decrease in the relative abundance of Mollicutes. UniFrac-based analysis of bacterial community membership shows an impact of diet on gut microbial ecology: cecal communities analyzed from two families of C57BL/6J wild-type mice (Table 13) generally cluster based on host diet (Western, FAT-R, and CARB-R). The average relative abundance (% of total 16S rRNA gene sequences) of bacterial lineages within the cecal microbiota of all mice fed a Western, FAT-R, or CARB-R diet is displayed as pie charts. Black boxes indicate nodes that were reproduced in >50% of all jackknife replications (n=126 sequences were randomly re-sampled). Asterisks indicate cecal samples that were analyzed by whole community shotgun sequencing.

FIG. 18 depicts charts showing the taxonomic assignments of metagenomic sequencing reads from seven cecal microbiome datasets based on BLAST homology searches, and by alignment of 16S rRNA gene fragments. (A) The cecal microbiome is dominated by sequences homologous to Bacteria. Sequencing reads were trimmed based on quality and vector sequence and the resulting datasets were used as queries against the NCBI non-redundant database (e-value<10-5). Sequences were assigned to the lowest taxonomic group that would include all significant hits, using MEGAN [18]. Pie charts are shown for each individual dataset and for the average of all datasets. Colors indicate assignments to bacteria (red), archaea (green), eukarya (yellow), viruses (blue), sequences that could not be confidently assigned to a group (purple), and sequences with no significant BLASTX matches (orange). (B) Relative abundance of microbiome sequences homologous to genomes from four bacterial divisions: Bacteroidetes (red), Proteobacteria (yellow), Actinobacteria (orange), and Firmicutes (blue). All divisions observed at >1% relative abundance are shown. (C) Relative abundance of microbiome sequences homologous to genomes from bacterial classes within the Firmicutes division: Bacilli (dark blue), Clostridia (yellow), and Mollicutes (light blue). (D) Taxonomic assignments of 16S rRNA gene fragments obtained from cecal microbiome datasets. 16S rRNA gene fragments were identified by querying the Ribosomal Database Project (RDP) database (version 9.33; BLASTN e-value <10-5) [34]. 16S rRNA gene fragments were aligned with NAST [41] and added to an ARB neighbor-joining tree [42]. 16S rRNA gene fragments from the Bacteroidetes (red), Proteobacteria (yellow), Verrucomicrobia (green), Mollicutes (light blue), and other Firmicutes (dark blue) are shown.

FIG. 19 depicts an illustration showing the metabolic reconstructions of the Eubacterium dolichum genome and the Western diet microbiome. Predicted gene presence calls for the Western diet microbiome and/or the E. dolichum genome are displayed in the upper right. Fermentation end-products and cellular biomass are highlighted in white ellipses. Note that culture based studies of E. dolichum have demonstrated its ability to produce lactate, acetate, and butyrate [37], suggesting that the apparent gap in the pathway for generating butyrate reflects the draft nature of the genome assembly or the possibility that this organism uses novel enzymes to generate this end-product of anaerobic fermentation. Abbreviations for enzymes (in boldface): Pgi, phosphoglucose isomerase; Pfk, phosphofructokinase; Fba, fructose-1,6-bisphosphate aldolase; Tpi, triose-phosphate isomerase; Gap, glyceraldehyde-3-phosphate dehydrogenase; Pgk, phosphoglycerate kinase; Pgm, phosphoglycerate mutase; Eno, enolase; Pyk, pyruvate kinase; EI, PTS enzyme I; HPr, PTS protein HPr; EIIA/B/C, PTS proteins; DXPS, 1-deoxy-D-xylulose-5-phosphate synthase; DXPR, DXP-reductoisomerase; MEPC, MEP cytidylyltransferase; MEK, CDPME kinase; MECS, MECDP-synthase; MDPS, 4-hydroxy-3-methylbut-2-en-1-yl diphosphate synthase; MDPR, 4-hydroxy-3-methylbut-2-enyl diphosphate reductase; Ldh, L-lactate dehydrogenase; Pfl, pyruvate formate-lyase; Pat, phosphate acetyltransferase; Ak, acetate kinase; Aca, acetyl-CoA C-acetyltransferase; Bhbd, 3-hydroxybutyryl-CoA dehydrogenase; Ech, enoyl-CoA hydratase; Bcd, butyryl-CoA dehydrogenase; Ptb, phosphotransbutyrylase; Bk, butyrate kinase; 1-Pfk, 1-phosphofructokinase; Npd, N-acetylglucosamine-6-phosphate deacetylase; Gpi, phosphoglucosamine isomerase; Fbf, fructan beta-fructosidase.

FIG. 20 depicts an illustration showing the assembly of metagenomic sequence data reveals physical linkage between the Mollicute phosphotransferase system (PTS) and other genes involved in carbohydrate metabolism. The pooled mouse gut microbiome dataset was assembled using ARACHNE [24] (n=7 combined datasets; see Tables S6 and S7 for assembly statistics). The contig length is shown as a solid black bar. Arrows indicate predicted proteins. Functional assignments were derived from the NCBI annotations and verified by BLASTP comparisons of each predicted protein with the STRING-extended COG database [19] and the KEGG database [20], in addition to Hidden Markov Model (HMM)-based protein domain searching with InterProScan [31]. Contigs 23 and 73 are >98% identical over the region in pink (234/238 nucleotides): they are likely different ends of the same gene that were not joined due to the relatively stringent assembly parameters employed.

FIG. 21 depicts a graph showing the concentration of bacterial fermentation end-products in the ceca of Western, FAT-R, and CARB-R mice. Acetate and butyrate levels (μmol per g wet weight cecal contents) were measured by gas chromatography mass spectrometry. Lactate levels (mM per kg protein) were measured using established microanalytic methods (see Examples). Data are represented as mean±SEM. Asterisks indicate significant differences (Student's t-test of Western versus CARB-R, *p<0.05, **p<0.01).

FIG. 22 depicts graphs showing principal component analysis (PCA) of sequenced Firmicute genomes. (A) PCA analysis of 14 previously sequenced Mollicute genomes (mostly Mycoplasma) and draft genome assemblies of nine human gut-associated Firmicutes (http://genome.wustl.edu/pub/). MetaGene was used to predict proteins from each genome [25]. Proteins were then assigned to KEGG orthologous groups based on homology (BLASTP e-value<10-5; KEGG version 40) [20]. Genomes were clustered based on the relative abundance of KEGG metabolic pathways (number of assignments to a given pathway divided by total number of pathway assignments). Only pathways found at >0.6% relative abundance in at least two genomes were included. The first two components are shown, representing 17% and 8% of the variance respectively. Abbreviations: Mca, Mycoplasma capricolum; Mfl, Mesoplasma forum L1; Mga, Mycoplasma gallisepticum R, Mge, Mycoplasma genitalium G37; Mhy232, Mycoplasma hyopneumoniae 232; Mhy7448, Mycoplasma hyopneumoniae 7448; MhyJ, Mycoplasma hyopneumoniae J; Mmo, Mycoplasma mobile 163K; Mmy, Mycoplasma mycoides subsp. mycoides SC str. PG1; Mpe, Mycoplasma penetrans HF-2; Mpn, Mycoplasmapneumoniae M129; Mpu, Mycoplasma pulmonis UAB CTIP; Msy, Mycoplasma synoviae 53; Upa, Ureaplasma parvum; E. dolichum, Eubacterium dolichum; CL250, Clostridium sp. L2-50; C. symbiosum, Clostridium symbiosum; Dlo, Dorea longicatena; Eel, Eubacterium eligens; Ere, Eubacterium rectale; Eve, Eubacterium ventriosum; Rob, Ruminococcus obeum; and Rto, Ruminococcus torques. (B) KEGG pathway relative abundance has a significant correlation with genome size. A linear regression was performed comparing PCA1 to genome size (or draft assembly size). PCA1 has a significant correlation to genome size (R2=0.9, p<0.05). (C) Metabolic pathways in E. dolichum. Pathways are marked partial if most genes are present and absent if genes are present.

FIG. 23 depicts the KEGG metabolic pathways significantly enriched in the human gut-derived Eubacterium dolichum strain DSM 3991 genome relative to eight human gut-associated Firmicutes. Pathways whose relative representation is significantly different between the E. dolichum genome and the pooled gut Firmicute genomes (n=8) were identified using a bootstrap comparison of the abundance of sequences assigned to all KEGG pathways (xipe version 2.4; confidence level=0.98, sample size=10,000) [32]. The relative abundance of all KEGG pathways with significantly different representation found at a relative abundance >0.6% in at least two microbiome datasets was transformed into a z-score and clustered by genome and pathway using a Euclidean distance metric [47]. Enrichment (yellow) and depletion (blue) are defined as a relative abundance greater or less than the mean for all datasets (i.e. a z-score greater or less than zero, respectively). For full strain names see FIG. 22.

FIG. 24 depicts a STRING-based protein network analysis of the predicted E. dolichum proteome. MetaGene [25] was used to predict proteins from the E. dolichum deep draft assembly. Proteins were subsequently assigned to COGs based on homology (BLASTP e-value<10⁻⁵) [19]. Annotated COG interactions were used to organize the protein network, including interactions based on neighborhood, gene fusion, co-occurrence, homology, co-expression, experiments, databases, and text mining (Medusa Java appet) [38]. Nodes, each representing a different orthologous group, are colored as follows: green, present in all analyzed Firmicute genomes (including the mycoplasma); blue, present in all recently sequenced gut Firmicute genomes; red, present in the Western dietassociated cecal microbiome (based on BLAST homology searches, e-value<10⁻⁵ and the deposited annotations in the STRING database, version 7). 89% of the COGs found in the E. dolichum genome were also found in the Western diet microbiome. Most of the COGs in green are involved in essential cellular functions such as transcription and translation (56% of the COG category assignments are to ‘Information storage and processing’). Some clusters of interest are highlighted, including the phosphotransferase system (PTS), the 2-methyl-D-erythritol 4-phosphate pathway for isoprenoid biosynthesis (MEP), cell wall biosynthesis, ABC transporters, and V-type ATPases for H⁺ import.

### SUMMARY OF THE INVENTION

One aspect of the present invention encompasses a method for decreasing energy harvesting, decreasing body fat, or for promoting weight loss in a subject. The method comprises altering the microbiota population in the subject's gastrointestinal tract by increasing the relative abundance of Bacteroidetes.

Another aspect of the invention encompasses a composition comprising an antibiotic having efficacy against Firmicutes but not against Bacteroidetes, and a probiotic comprising Bacteroidetes.

Yet another aspect of the invention encompasses a method for selecting a compound for treating obesity or an obesity-related disorder in a host. The method comprises providing a microbiome profile from the host and providing a plurality of reference microbiome profiles, each associated with a compound. The host profile and each reference profile has a plurality of values, each value representing the abundance of a microbiome biomolecule. The method further comprises selecting the reference profile most similar to the host microbiome profile, thereby selecting a compound for treating obesity or an obesity-related disorder in the host.

Still another aspect of the invention encompasses a method to determine whether a compound has efficacy for treatment of obesity or an obesity-related disorder in a host. The method comprises comparing a plurality of biomolecules of the host's microbiome before and after administration of a drug for the treatment of obesity, such that if the abundance of biomolecules associated with obesity decreased after treatment, the compound is efficacious in treating obesity in a host.

An additional aspect of the invention encompasses a method of predicting risk for obesity or an obesity-related disorder in a host. The method comprises providing a microbiome profile from said host and providing a plurality of reference microbiome profiles. The host profile and each reference profile has a plurality of values, each value representing the abundance of a microbiome biomolecule. The method further comprises selecting the reference profile most similar to the host microbiome profile, such that if the host's microbiome is most similar to a reference obese microbiome, the host is at risk for obesity or an obesity-related disorder.

Another additional aspect of the invention encompasses a computer-readable medium comprising a plurality of digitally encoded profiles wherein each profile of the plurality has a plurality of values, each value representing the abundance of a biomolecule in an obese host microbiome.

A further aspect of the invention encompasses a kit for evaluating a drug, or for diagnosing or prognosing a gut microbiome associated with increased energy harvesting, increased body fat, and/or weight gain. The kit comprises an array comprising a substrate, the substrate having disposed thereon at least one biomolecule that is modulated in an obese host microbiome compared to a lean host microbiome, and a computer-readable medium having a plurality of digitally-encoded profiles wherein each profile of the plurality has a plurality of values, each value representing the abundance of biomolecule in a host microbiome detected by the array.

Another further aspect of the invention encompasses at method for decreasing body fat or for promoting weight loss in a subject. The method comprising altering the activity of the microbiota population in the subject's gastrointestinal tract by altering the microbiota population.

Other aspects and iterations of the invention are described more thoroughly below.

### DETAILED DESCRIPTION OF THE INVENTION

It has been discovered, as demonstrated in the Examples, that there is a relationship between the diversity of the gut microbiota and obesity. In particular, an obese subject typically has fewer Bacteroidetes and more Firmicutes compared to a lean subject. Taking advantage of these discoveries, the present invention provides compositions and methods to regulate energy balance in a subject. The invention also provides tools utilizing the gut microbiome as a diagnostic or prognostic biomarker for obesity risk, a biomarker for drug discovery, a biomarker for the discovery of therapeutic targets involved in the regulation of energy balance, and a biomarker for the efficacy of a weight loss program.

### I. Modulation of Energy Balance in a Subject

The energy balance of a subject may be modulated by altering the subject's gut microbiota population. Generally speaking, to decrease energy harvesting, decrease body fat, or promote weight loss, the relative abundance of bacteria within the Bacteroidetes division is increased and optionally, the relative abundance of bacteria within the Firmicutes division is decreased. Alternatively, to increase energy harvesting, to increase body fat, or promote weight gain, the relative abundance of Bacteroidetes is decreased and optionally, the relative abundance of Firmicutes is increased. Additional agents may also be utilized to achieve either weight loss or weight gain. Examples of these agents are detailed in section I(c).

(a) Altering the abundance of Bacteroides and/or Firmicutes

The relative abundance of Bacteroidetes may be altered by increasing or decreasing the presence of one or more Bacteroidetes species that reside in the gut. Non-limiting examples of species may include the species listed in Table A. Additionally, non-limiting examples of species may include B. thetaiotaomicron, B. vulgatus, B. ovatus, B. distasonis, B. uniformis, B. stercoris, B. eggerthii, B. merdae, and B. caccae. In one embodiment, the population of B. thetaiotaomicron is altered. In still another embodiment, the population of B. vulgatus is altered. In an additional embodiment, the population of B. ovatus is altered. In another embodiment, the population of B. distasonis is altered. In yet another embodiment, the population of B. uniformis is altered. In an additional embodiment, the population of B. stercoris is altered. In a further embodiment, the population of B. eggerthii is altered. In still another embodiment, the population of B. merdae is altered. In another embodiment, the population of B. caccae is altered. In a further embodiment, the species within the division Bacteroidetes may be as of yet unnamed.

TABLE A

| Number | Divisions | Genus | Species | Strain ID |
|---|---|---|---|---|
| 1 | Bacteroidetes | Alistepes | putredinis | ATCC 29800 |
| 2 | Bacteroidetes | Bacteroides | caccae | ATCC 43185T |
| 3 | Firmicutes | Clostridium | leptum | ATCC 29065 |
| 4 | Firmicutes | Clostridium | boltaea | ATCC BAA-613 |
| 5 | Firmicutes | Peptostreptococcus | micros | ATCC 33270 |
| 6 | Firmicutes | Eubacterium | ventriosum | ATCC 27560 |
| 7 | Firmicutes | Eubacterium | halii | ATCC 27751 |
| 8 | Firmicutes | Ruminococcus | gnavus | ATCC 29149 |
| 9 | Firmicutes | Coprococcus | catus | ATCC 27761 |
| 10 | Firmicutes | Eubacterium | siraeum | ATCC 29066 |
| 11 | Firmicutes | Ruminococcus | obeum | ATCC 29174 |
| 12 | Firmicutes | Ruminococcus | torques | ATCC 27756 |
| 13 | Firmicutes | Subdoligranulum | variabile | CCUG 47106 |
| 14 | Firmicutes | Dorea | formicigenerans | ATCC 27755 |
| 15 | Firmicutes | Dorea | longicatena | CCUG 45247 |
| 16 | Firmicutes | Faecalibacterium | prausnitzii | ATCC 27768 |
| 17 | Bacteroidetes | Bacteroides | sp. | CCUG 39913 |
| 18 | Bacteroidetes | Bacteroides | sp. | Smarlab 3301186 |
| 19 | Bacteroidetes | Bacteroides | ovatus | ATCC 8483T |
| 20 | Bacteroidetes | Bacteroides | salyersiae | ATCC BAA-997 |
| 21 | Bacteroidetes | Alistepes | finegoldii | CCUG 46020 |
| 22 | Bacteroidetes | Bacteroides | sp. | MPN isolate group 6 |
| 23 | Bacteroidetes | Bacteroides | sp. | DSM 12148 |
| 24 | Bacteroidetes | Bacteroides | merdae | ATCC 43184T |
| 25 | Bacteroidetes | Bacteroides | stercosis | ATCC 43183T |
| 26 | Bacteroidetes | Bacteroides | uniformis | ATCC 8492 |
| 27 | Bacteroidetes | Bacteroides | WH302 | Gordon Lab |
| 28 | Firmicutes | Bulleidia | moorei | ATCC BAA-170 |
| 29 | Firmicutes | Bacteroides | capillosus | ATCC 29799 |
| 30 | Firmicutes | Ruminococcus | bromii | ATCC 27255 |
| 31 | Firmicutes | Clostridium | symbiosum | ATCC 14940 |
| 32 | Firmicutes | Clostridium | sp. | DSM 6877(FS41) |
| 33 | Firmicutes | Clostridium | sp. | A2-207 |
| 34 | Firmicutes | Anaerofustis | stercorihominis | CCUG 47767T |
| 35 | Firmicutes | Clostridium | scindens | ATCC 35704 |
| 36 | Firmicutes | Clostridium | spiroforme | DSM 1552 |
| 37 | Firmicutes | Ruminococcus | callidus | ATCC 27760 |
| 38 | Firmicutes | Coprococcus | eutactus | ATCC 27759 |
| 39 | Firmicutes | Gemella | haemolysans | ATCC 10379 |
| 40 | Firmicutes | Clostridium | sp. | A2-183 |
| 41 | Firmicutes | Clostridium | sp. | SL6/1/1 |
| 42 | Firmicutes | Roseburia | intestinalis | DSM 14610 |
| 43 | Firmicutes | Clostridium | sp. | GM2/1 |
| 44 | Firmicutes | Clostridium | sp. | A2-194 |
| 45 | Firmicutes | Clostridium | sp. | 14774 |
| 46 | Firmicutes | Clostridium | sp. | A2-166 |
| 47 | Firmicutes | Clostridium | sp. | A2-175 |
| 48 | Firmicutes | Roseburia | faecalis | M6/1 |
| 49 | Firmicutes | Catenibacterium | mitsuokai | JCM 10609 |
| 50 | Firmicutes | Clostridium | sp. | SR1/1 |
| 51 | Firmicutes | Clostridium | sp. | L1-83 |
| 52 | Firmicutes | Clostridium | sp. | L2-6 |
| 53 | Firmicutes | Clostridium | sp. | A2-231 |
| 54 | Firmicutes | Clostridium | sp. | A2-165 |
| 55 | Firmicutes | Dialister | sp. | E2_20 |
| 56 | Firmicutes | Clostridium | sp. | SS2/1 |
| 57 | Firmicutes | Anaerotruncus | colihominis | CCUG 45055T |
| 58 | Firmicutes | Eubacterium | plautii | ATCC 29863 |
| 59 | Firmicutes | Clostridium | bartlettii | CCUG 48940 |
| 60 | Firmicutes | Lactobacilllus | lactis | Ssp. IL1403 |

The present invention also includes altering various combinations of species, such as at least two species, at least three species, at least four species, at least five species, at least six species, at least seven species, at least eight species, at least nine species, or at least ten species. For example, the combination of B. thetaiotaomicron, B. vulgatus, B. ovatus, B. distasonis, and B. uniformis may be altered.

In an exemplary embodiment, the relative abundance of Bacteroidetes is increased to decrease energy harvesting, decrease body fat, or promote weight loss in a subject. Increased abundance of Bacteroidetes in the gut may be accomplished by several suitable means generally known in the art. In one embodiment, a food supplement that increases the abundance of Bacteroidetes may be administered to the subject. By way of example, one such food supplement is psyllium husks as described in U.S. Patent Application Publication No. 2006/0229905, which is hereby incorporated by reference in its entirety. In an exemplary embodiment, a probiotic comprising Bacteroidetes may be administered to the subject. The amount of probiotic administered to the subject can and will vary depending upon the embodiment. The probiotic may be present at a level of from about one thousand to about ten billion cfu/g (colony forming units per gram) of the total composition or of the part of the composition comprising the probiotic. In one embodiment, the probiotic may be present at a level of from about one hundred million to about 10 billion organisms. The probiotic microorganism may be in any suitable form, for example in a powdered dry form. In addition, the probiotic microorganism may have undergone processing in order for it to increase its survival. For example, the microorganism may be coated or encapsulated in a polysaccharide, fat, starch, protein or in a sugar matrix. Standard encapsulation techniques known in the art can be used, and for example, as discussed in U.S. Pat. No. 6,190,591, which is hereby incorporated by reference in its entirety.

Alternatively, the relative abundance of Bacteroidetes is decreased to increase energy harvesting, increase body fat, or promote weight gain in a subject. Decreased abundance of Bacteroidetes in the gut may be accomplished by several suitable means generally known in the art. In one embodiment, an antibiotic having efficacy against Bacteroidetes may be administered. Generally speaking, antimicrobial agents may target several areas of bacterial physiology: protein translation, nucleic acid synthesis, folic acid metabolism, or cell wall synthesis. In an exemplary embodiment, the antibiotic will have efficacy against Bacteriodetes but not against Firmicutes. The susceptibility of the targeted species to the selected antibiotics may be determined based on culture methods or genome screening.

It is contemplated that the abundance of gut Bacteroidetes within an individual subject may be altered (i.e., increased or decreased) from about a one fold difference to about a ten fold difference or more, depending on the desired result (i.e., increased energy harvesting (weight gain) or decreased energy harvesting (weight loss)) and the individual subject. In one embodiment, the abundance may be altered from about a one fold difference to about a ten fold difference. For weight loss, the abundance may be altered by an increase of about a two fold difference to about a ten fold difference, of about a three fold difference to about a ten fold difference, of about a four fold difference to about a ten fold difference, of about a five fold difference to about a ten fold difference, or of about a six fold difference to about a ten fold difference. A method for determining the relative abundance of gut Bacteroidetes is described in the examples, alternatively, an array of the invention, described below, may be used to determine the relative abundance.

Stated another way, it is contemplated that the abundance of gut Bacteroidetes within an individual subject may be altered (i.e., increased or decreased) from about 1% to about 100% or more depending on the desired result (i.e., increased energy harvesting (weight gain) or decreased energy harvesting (weight loss)) and the individual subject. For weight loss, the abundance may be altered by an increase of from about 20% to about 100%, from about 30% to about 100%, from about 40% to about 100%, from about 50% to about 100%, from about 60% to about 100%, from about 70% to about 100%, from about 80% to about 100%, or from about 90% to 100%. A method for determining the relative abundance of gut Bacteroidetes is described in the examples, alternatively, an array of the invention, described below, may be used to determine the relative abundance.

### (b) Altering the Abundance of Firmicutes

The relative abundance of Firmicutes may be altered by increasing or decreasing the presence of one or more species that reside in the gut. Non-limiting examples of species may include the species listed in Table A Representative species include species from Clostridia, Bacilli, and Mollicutes. In one embodiment, the relative abundance of one or more Clostridia species is altered. In another embodiment, the relative abundance of one or more Bacilli species is altered. In yet another embodiment, the relative abundance of one or more Mollicutes species is altered. It is also contemplated that the relative abundance of several species of Firmicutes may be altered without departing from the scope of the invention. By way of non-limiting examples, a combination of one or more Clostridia species, one or more Bacilli species, and one or more Mollicutes species may be altered. In a further embodiment, the species within the division Firmicutes may be as of yet unnamed.

In some embodiments, the Mollicutes class is altered. For instance, E. dolichum, E. cylindroides, or E. biforme may be altered. In one embodiment, the species of the Mollicutes class may posses the genetic information to create a cell wall. In another embodiment, the species of the Mollicutes class may produce a cell wall. In a further embodiment, the species within the class Mollicutes may be as of yet unnamed.

In an exemplary embodiment, the relative abundance of Firmicutes is decreased to decrease energy harvesting, decrease body fat, or promote weight loss in a subject. Decreased abundance of Firmicutes in the gut may be accomplished by several suitable means generally known in the art. In one embodiment, an antibiotic having efficacy against Firmicutes may be administered. In an exemplary embodiment, the antibiotic will have efficacy against Firmicutes but not against Bacteriodetes. In another exemplary embodiment, the antibiotic will have efficacy against Mollicutes, but not Bacteriodetes. The susceptibility of the targeted species to the selected antibiotics may be determined based on culture methods or genome screening.

Alternatively, the relative abundance of Firmicutes is increased to increase energy harvesting, increase body fat, or promote weight gain in a subject. Increased abundance of Firmicutes in the gut may be accomplished by several suitable means generally known in the art. In an exemplary embodiment, a probiotic comprising Firmicutes may be administered to the subject.

It is contemplated that the abundance of gut Firmicutes may be altered (i.e., increased or decreased) from about a one fold difference to about a ten fold difference or more, depending on the desired result (i.e., increased energy harvesting (weight gain) or decreased energy harvesting (weight loss)). For weight loss, the abundance may be altered by a decrease of about a one fold difference to about a ten fold difference, a two fold difference to about a ten fold difference, of about a three fold difference to about a ten fold difference, of about a four fold difference to about a ten fold difference, of about a five fold difference to about a ten fold difference, or of about a six fold difference to about a ten fold difference. A method for determining the relative abundance of gut Firmicutes is described in the examples.

Stated another way, it is contemplated that the abundance of gut Firmicutes may be altered (i.e., increased or decreased) from about 1% to about 100% or more depending on the desired result (i.e., increased energy harvesting (weight gain) or decreased energy harvesting (weight loss)). For weight loss, the abundance may be altered by a decrease of from about 20% to about 100%, from about 30% to about 100%, from about 40% to about 100%, from about 50% to about 100%, from about 60% to about 100%, from about 70% to about 100%, from about 80% to about 100%, or from about 90% to 100%. A method for determining the relative abundance of gut Firmicutes is described in the examples.

### (c) Additional Weight Modulating Agents

Another aspect of the invention encompasses a combination therapy to regulate fat storage, energy harvesting, and/or weight loss or gain in a subject. In an exemplary embodiment, a combination for decreasing energy harvesting, decreasing body fat or for promoting weight loss is provided. For this embodiment, a composition comprising an antibiotic having efficacy against Firmicutes but not against Bacteroidetes; and a probiotic comprising Bacteroidetes may be administered to the subject. Additionally, an anti-archea compound may be included in the aforementioned composition. Other agents that may be included with the aforementioned composition are detailed below.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means. The actual effective amounts of compounds comprising a weight loss composition of the invention can and will vary according to the specific compounds being utilized, the mode of administration, and the age, weight and condition of the subject. Dosages for a particular individual subject can be determined by one of ordinary skill in the art using conventional considerations. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

i. Fiaf Polypeptide

A composition of the invention for promoting weight loss may optionally include either increasing the amount of a Fiaf polypeptide or the activity of a Fiaf polypeptide. Typically, a suitable Fiaf polypeptide is one that can substantially inhibit LPL when administered to the subject. Several Fiaf polypeptides known in the art are suitable for use in the present invention. Generally speaking, the Fiaf polypeptide is from a mammal. By way of non-limiting example, suitable Fiaf polypeptides and nucleotides are delineated in Table B.

TABLE B

| Species | PubMed Ref. |
|---|---|
| Homo sapiens | NM_139314 |
| | NM_016109 |
| Mus musculus | NM_020581 |
| Rattus norvegicus | NM_199115 |
| Sus scrofa | AY307772 |
| Bos taurus | AY192008 |
| Pan troglodytes | AY411895 |

In certain aspects, a polypeptide that is a homolog, ortholog, mimic or degenerative variant of a Fiaf polypeptide is also suitable for use in the present invention. In particular, the subject polypeptide will typically inhibit LPL when administered to the subject. A variety of methods may be employed to determine whether a particular homolog, mimic or degenerative variant possesses substantially similar biological activity relative to a Fiaf polypeptide. Specific activity or function may be determined by convenient in vitro, cell-based, or in vivo assays, such as measurement of LPL activity in white adipose tissue or in the heart. In order to determine whether a particular Fiaf polypeptide inhibits LPL, the procedure detailed in the examples of U.S. Patent Application No. 20050239706, which is hereby incorporated by reference in its entirety, may be followed.

Fiaf polypeptides suitable for use in the invention are typically isolated or pure and are generally administered as a composition in conjunction with a suitable pharmaceutical carrier, as detailed below. A pure polypeptide constitutes at least about 90%, preferably, 95% and even more preferably, at least about 99% by weight of the total polypeptide in a given sample.

The Fiaf polypeptide may be synthesized, produced by recombinant technology, or purified from cells using any of the molecular and biochemical methods known in the art that are available for biochemical synthesis, molecular expression and purification of the Fiaf polypeptides [see e.g., Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York)].

The invention also contemplates use of an agent that increases Fiaf transcription or its activity. For example, an agent may be delivered that specifically activates Fiaf expression: this agent may be a natural or synthetic compound that directly activates Fiaf gene transcription, or indirectly activates expression through interactions with components of host regulatory networks that control Fiaf transcription. Suitable agents may be identified by methods generally known in the art, such as by screening natural product and/or chemical libraries using the gnotobiotic zebrafish model described in the examples of U.S. Patent Application No. 20050239706. In another embodiment, a chemical entity may be used that interacts with Fiaf targets, such as LPL, to reproduce the effects of Fiaf (e.g., in this case inhibition of LPL activity). In an alternative of this embodiment, administering a Fiaf agonist to the subject may increase Fiaf expression and/or activity. In one embodiment, the Fiaf agonist is a peroxisome proliferator-activated receptor (PPARs) agonist. Suitable PPARs include PPARα, PPARβ/δ, and PPARγ. Fenofibrate is another suitable example of a Fiaf agonist. Additional suitable Fiaf agonists and methods of administration are further described in Manards, et al., J. Biol Chem, 279, 34411 (2004), and U.S. Patent Publication No. 2003/0220373, which are both hereby incorporated by reference in their entirety.

ii. Other Compounds

The compositions of the invention that decrease energy harvesting, decrease body fat, or promote weight loss may also include several additional agents suitable for use in weight loss regimes. Generally speaking, exemplary combinations of therapeutic agents may act synergistically to decrease energy harvesting, decrease body fat, or promote weight loss. Using this approach, one may be able to achieve therapeutic efficacy with lower dosages of each agent, thus reducing the potential for adverse side effects. In one embodiment, acarbose may be administered with a composition of the invention. Acarbose is an inhibitor of α-glucosidases and is required to break down carbohydrates into simple sugars within the gastrointestinal tract of the subject. In another embodiment, an appetite suppressant, such as an amphetamine, or a selective serotonin reuptake inhibitor, such as sibutramine, may be administered with a composition of the invention. In still another embodiment, a lipase inhibitor such as orlistat, or an inhibitor of lipid absorption such as Xenical, may be administered with a composition of the invention.

iii. Restricted Calorie Diet

Optionally, in addition to administration of a composition of the invention for weight loss, a subject may also be placed on a restricted calorie diet. As shown in the example, restricted calorie diets are helpful for increasing the relative abundance of Bacteroidetes and decreasing the relative abundance of Firmicutes. Several restricted calorie diets known in the art are suitable for use in combination with the compositions of the invention. Representative diets include a reduced fat diet, reduced protein, or a reduced carbohydrate diet.

iv. Alteration of the Gastrointestinal Archaeon Population

An anti-archea compound may be included in a composition of the invention to decrease energy harvesting, decrease fat storage, and/or decrease weight gain. To promote weight loss in a subject, the archaeon population is altered such that microbial-mediated carbohydrate metabolism or its efficiency is decreased in the subject, whereby decreasing microbial-mediated carbohydrate metabolism or its efficiency promotes weight loss in the subject.

Accordingly, in one embodiment, the subject's gastrointestinal archaeon population is altered so as to promote weight loss in the subject. Typically, the presence of at least one genera of archaeon that resides in the gastrointestinal tract of the subject is decreased. In most embodiments, the archaeon is generally a mesophilic methanogenic archaea. In one alternative of this embodiment, the presence of at least one species from the genera Methanobrevibacter or Methanosphaera is decreased. In another alternative embodiment, the presence of Methanobrevibacter smithii is decreased. In still another embodiment, the presence of Methanosphaera stadtmanae is decreased. In yet another embodiment, the presence of a combination of archaeon genera or species is decreased. By way of non-limiting example, the presence of Methanobrevibacter smithii and Methanosphaera stadtmanae is decreased.

To decrease the presence of any of the archaeon detailed above, methods generally known in the art may be utilized. In one embodiment, a compound having anti-microbial activities against the archaeon is administered to the subject. Non-limiting examples of suitable anti-microbial compounds include metronidzaole, clindamycin, timidazole, macrolides, and fluoroquinolones. In another embodiment, a compound that inhibits methanogenesis by the archaeon is administered to the subject. Non-limiting examples include 2-bromoethanesulfonate (inhibitor of methyl-coenzyme M reductase), N-alkyl derivatives of para-aminobenzoic acid (inhibitor of tetrahydromethanopterin biosynthesis), ionophore monensin, nitroethane, lumazine, propynoic acid and ethyl 2-butynoate. In yet another embodiment, a hydroxymethylglutaryl-CoA reductase inhibitor is administered to the subject. Non-limiting examples of suitable hydroxymethylglutaryl-CoA reductase inhibitors include lovastatin, atorvastatin, fluvastatin, pravastatin, simvastatin, and rosuvastatin. Alternatively, the diet of the subject may be formulated by changing the composition of glycans (e.g., polyfructose-containing oligosaccharides) in the diet that are preferred by polysaccharide degrading bacterial components of the microbiota (e.g., Bacteroides spp) when in the presence of mesophilic methanogenic archaeal species such as Methanobrevibacter smithii.

Generally speaking, when the archaeon population in the subject's gastrointestinal tract is decreased in accordance with the methods described above, the polysaccharide degrading properties of the subject's gastrointestinal microbiota is altered such that microbial-mediated carbohydrate metabolism or its efficiency is decreased. Typically, depending upon the embodiment, the transcriptome and the metabolome of the gastrointestinal microbiota is altered. In one embodiment, the microbe is a saccharolytic bacterium. In one alternative of this embodiment, the saccharolytic bacterium is a Bacteroides species. In a further alternative embodiment, the bacterium is Bacteroides thetaiotaomicron. Typically, the carbohydrate will be a plant polysaccharide or dietary fiber. Plant polysaccharides include starch, fructan, cellulose, hemicellulose, and pectin.

The compounds utilized in this invention to alter the archaeon population may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

The actual effective amounts of compound described herein can and will vary according to the specific composition being utilized, the mode of administration and the age, weight and condition of the subject. Dosages for a particular individual subject can be determined by one of ordinary skill in the art using conventional considerations. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

### II. Biomarkers Comprising the Gut Microbiome

Another aspect of the invention encompasses use of the gut microbiome as a biomarker for obesity. The biomarker may be utilized to construct arrays that may be used for several applications including as a diagnostic or prognostic tool to determine obesity risk, judging efficacy of existing weightloss regimes, drug discovery, for the identification of additional biomarkers involved in obesity or an obesity related disorder, and for the discovery of therapeutic targets involved in the regulation of energy balance. Generally speaking, the array may comprise biomolecules from an obese host microbiome, including a diet-induced obese host microbiome, or a lean host microbiome.

### (a) Array

The array may be comprised of a substrate having disposed thereon at least one biomolecule that is modulated in an obese host microbiome compared to a lean host microbiome. Several substrates suitable for the construction of arrays are known in the art, and one skilled in the art will appreciate that other substrates may become available as the art progresses. The substrate may be a material that may be modified to contain discrete individual sites appropriate for the attachment or association of the biomolecules and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an exemplary embodiment, the substrates may allow optical detection without appreciably fluorescing.

A substrate may be planar, a substrate may be a well, i.e. a 364 well plate, or alternatively, a substrate may be a bead. Additionally, the substrate may be the inner surface of a tube for flow-through sample analysis to minimize sample volume. Similarly, the substrate may be flexible, such as a flexible foam, including closed cell foams made of particular plastics.

The biomolecule or biomolecules may be attached to the substrate in a wide variety of ways, as will be appreciated by those in the art. The biomolecule may either be synthesized first, with subsequent attachment to the substrate, or may be directly synthesized on the substrate. The substrate and the biomolecule may be derivatized with chemical functional groups for subsequent attachment of the two. For example, the substrate may be derivatized with a chemical functional group including, but not limited to, amino groups, carboxyl groups, oxo groups or thiol groups. Using these functional groups, the biomolecule may be attached using functional groups on the biomolecule either directly or indirectly using linkers.

The biomolecule may also be attached to the substrate non-covalently. For example, a biotinylated biomolecule can be prepared, which may bind to surfaces covalently coated with streptavidin, resulting in attachment. Alternatively, a biomolecule or biomolecules may be synthesized on the surface using techniques such as photopolymerization and photolithography. Additional methods of attaching biomolecules to arrays and methods of synthesizing biomolecules on substrates are well known in the art, i.e. VLSIPS technology from Affymetrix (e.g., see U.S. Pat. No. 6,566,495, and Rockett and Dix, “DNA arrays: technology, options and toxicological applications,” Xenobiotica 30(2):155-177, all of which are hereby incorporated by reference in their entirety).

In one embodiment, the biomolecule or biomolecules attached to the substrate are located at a spatially defined address of the array. Arrays may comprise from about 1 to about several hundred thousand addresses. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment, the array may be comprised of at least 5,000 addresses. In a further embodiment, the array may be comprised of less than 500 addresses. In yet a further embodiment, the array may be comprised of at least 500 addresses.

A biomolecule may be represented more than once on a given array. In other words, more than one address of an array may be comprised of the same biomolecule. In some embodiments, two, three, or more than three addresses of the array may be comprised of the same biomolecule. In certain embodiments, the array may comprise control biomolecules and/or control addresses. The controls may be internal controls, positive controls, negative controls, or background controls.

The array may be comprised of biomolecules indicative of an obese host microbiome. Alternatively, the array may be comprised of biomolecules indicative of a lean host microbiome. A biomolecule is “indicative” of an obese or lean microbiome if it tends to appear more often in one type of microbiome compared to the other. Additionally, the array may be comprised of biomolecules that are modulated in the obese host microbiome compared to the lean host microbiome. As used herein, “modulated” may refer to a biomolecule whose representation or activity is different in an obese host microbiome compared to a lean host microbiome. For instance, modulated may refer to a biomolecule that is enriched, depleted, up-regulated, down-regulated, degraded, or stabilized in the obese host microbiome compared to a lean host microbiome.

In one embodiment, the array may be comprised of a biomolecule enriched in the obese host microbiome compared to the lean host microbiome. In another embodiment, the array may be comprised of a biomolecule depleted in the obese host microbiome compared to the lean host microbiome. In yet another embodiment, the array may be comprised of a biomolecule up-regulated in the obese host microbiome compared to the lean host microbiome. In still another embodiment, the array may be comprised of a biomolecule down-regulated in the obese host microbiome compared to the lean host microbiome. In still yet another embodiment, the array may be comprised of a biomolecule degraded in the obese host microbiome compared to the lean host microbiome. In an alternative embodiment, the array may be comprised of a biomolecule stabilized in the obese host microbiome compared to the lean host microbiome.

Generally speaking, an array of the invention may comprise at least one biomolecule indicative or, or modulated in, an obese host microbiome compared to a lean host microbiome. In one embodiment, the array may comprise at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 biomolecules indicative of, or modulated in, an obese host microbiome compared to a lean host microbiome. In another embodiment, the array may comprise at least 200, at least 300, at least 400, at least 500, or at least 600 biomolecules indicative of, or modulated in, an obese host microbiome compared to a lean host microbiome.

As used herein, “biomolecule” may refer to a nucleic acid, an oligonucleic acid, an amino acid, a peptide, a polypeptide, a protein, a lipid, a metabolite, or a fragment thereof. Nucleic acids may include RNA, DNA, and naturally occurring or synthetically created derivatives. A biomolecule may be present in, produced by, or modified by a microorganism within the gut.

Biomolecules that are enriched in the obese microbiome compared to the lean microbiome may include biomolecules derived from the following Kyoto Encyclopedia of Genes and Genomes (KEGG) Categories: Carbohydrate Metabolism, Amino Acid Metabolism, Metabolism of Other Amino Acids, Glycan Biosynthesis and Metabolism, Biosynthesis of Polyketides and Nonribosomal Peptides, Transcription, Folding/Sorting/Degradation, Signal Transduction, and Cell Growth and Death. In certain embodiments, the biomolecules derived from the KEGG categories above may include biomolecules from a corresponding KEGG pathway (see Examples). Additionally, biomolecules that are enriched in the obese microbiome compared to the lean microbiome may include nucleic acids encoding proteins or portions of proteins derived from the following Clusters of Orthologous Genes (COGs): Transcription, Replication/recombination/repair, Nuclear structure, signal transduction, cell wall/membrane/envelope biogenesis, Energy production, Nucleotide, Ion, and cell motility.

Alternatively, biomolecules that are depleted in the obese microbiome compared to the lean microbiome may be biomolecules derived from the following KEGG categories: Carbohydrate Metabolism, Energy Metabolism, Lipid Metabolism, Nucleotide Metabolism, Amino Acid Metabolism, Glycan Biosynthesis and Metabolism, Metabolism of Cofactors and Vitamins, Translation, and Folding/Sorting/Degradation. In certain embodiments, the biomolecules encoding proteins or portions of proteins derived from the KEGG categories above may include biomolecules from a corresponding KEGG pathway (see Examples). Additionally, biomolecules that are depleted in the obese microbiome compared to the lean microbiome may include biomolecules encoding proteins or portions of proteins derived from the following COGs: Translation, Defense Mechanisms, Energy Production, Nucleotide, Coenzyme, Ion, and Posttranslational modification/protein turnover/chaperones.

Biomolecules indicative of, or modulated in, an obese host microbiome compared to a lean host microbiome may include biomolecules associated with di- and poly-saccharide (fructoside) degradation, such as ‘fructan beta-fructosidase’ (K03332), a gene that allows the degradation of sucrose, inulin, and/or levan, or a biomolecule associated with the KEGG pathway for fructose and mannose metabolism. Additionally, the array may include biomolecules associated with the import of mono- and di-saccharides via the Phosphotransferase system (PTS), such as biomolecules for importing and metabolizing fructose, glucose, N-acetyl-glucosamine, and N-acetyl-galactosamine. Also, the array may include biomolecules associated with the Metabolism of imported carbohydrates, such as biomolecules associated with the KEGG pathway for Glycolysis, including biomolecules to process imported carbohydrates to phosphoenolpyruvate (PEP). The array may further include biomolecues associated with anaerobic fermentation, such as biomolecules associated with the pathways for the fermentation of carbohydrates to acetate, butyrate, and lactate. In each of the above embodiments, the biomolecules are indicative of, or modulated in, an obese host microbiome compared to a lean host microbiome.

In some embodiments, the biomolecules of the array may be selected from biomolecules involved in polysaccharide degradation. For instance, the array may comprise biomolecules involved in polysaccharide degradation that are indicative of, or modulated in, an obese host microbiome compared to a lean host microbiome. In particular, the array may comprise glycoside hydrolases that are indicative of, or modulated in, an obese host microbiome compared to the lean host microbiome. In one embodiment, the array may comprise biomolecules from the CAZy familes 2, 4, 27, 31, 35, 36, 42, and 68 that are indicative of or modulated in an obese host microbiome compared to a lean host microbiome. In another embodiment, the array may comprise biomolecules from the CAZy families 2, 4, 27, 31, 35, 36, 42, and 68 that are up-regulated or enriched in an obese host microbiome compared to a lean host microbiome. The CAZy database describes the families of structurally-related catalytic and carbohydrate-binding modules (or functional domains) of enzymes that degrade, modify, or create glycosidic bonds, and may be accessed at http://www.cazy.org/index.html. In another embodiment, the array may comprise alpha-galactosidases, beta-galactosidases, alpha-amylases and amylomaltases that are indicative of, or modulated in, an obese host microbiome compared to a lean host microbiome. Additionally, the array may comprise biomolecules selected from the KEGG pathways for starch and sucrose metabolism, galactose metabolism, and butanoate metabolism that are indicative of, or modulated in, an obese host microbiome compared to a lean host microbiome (See Tables Z, Y, and X).

In other embodiments, the biomolecules of the array may be selected from biomolecules involved in carbohydrate import that are indicative of, or modulated in, an obese host microbiome compared to a lean host microbiome. For instance, the biomolecules may be ABC transporters (See Table V). In yet another embodiment, the biomolecules may be selected from biomolecules involved in acetogenesis, or the generation of acetate from CO₂ (See Table W). For instance, the biomolecule may be a formate-tetrahydrofolate ligase.

In still other embodiments, the biomolecules may be selected from biomolecules involved in anaerobic fermentation that are indicative of, or modulated in, an obese host microbiome compared to a lean host microbiome. For instance, the biomolecules may be selected from biomolecules involved in the fermentation of carbohydrates to acetate and butyrate. Specifically, the biomarker may comprise pyruvate formate-lyase. Alternatively, the biomarker may comprise biomolecules in the KEGG butanoate metabolism pathway (See Table X).

In certain embodiments, the biomolecules of the array may be selected from the nucleic acid sequences represented by GenBank project accession numbers AATA00000000-AATF00000000, i.e. including the AATB, AATC, AATD, and AATE accession numbers. Alternatively, the biomolecules may be selected from the proteins encoded by the nucleic acid sequences represented by GenBank project accession numbers AATA00000000-AATF00000000, i.e. including the AATB, AATC, AATD, and AATE accession numbers. In some embodiments, the biomolecules may be selected from the nucleic acid sequences represented by GenBank project accession numbers AATA00000000-AATF00000000, i.e. including the AATB, AATC, AATD, and AATE accession numbers that are modulated in the obese host microbiome compared to the lean host microbiome. In another alternative, the biomolecules may be selected from the proteins encoded by the nucleic acid sequences represented by GenBank project accession numbers AATA00000000-AATF00000000, i.e. including the AATB, AATC, AATD, and AATE accession numbers that are modulated in the obese host microbiome compared to the lean host microbiome.

In several embodiments, the biomolecules of the array may be selected from the biomolecules represented by the accession numbers listed in Tables Z-V. Table Z represents the accession numbers of 629 biomolecules involved in starch and sucrose metabolism that are enriched in the obese host microbiome compared to the lean host microbiome. Table Y represents the accession numbers of 205 biomolecules involved in galactose metabolism that are enriched in the obese host microbiome compared to the lean host microbiome. Table X represents the accession numbers of 124 biomolecules involved in butanoate metabolism that are enriched in the obese host microbiome compared to the lean host microbiome. Table W represents the accession numbers of 14 biomolecules involved in acetogenesis that are enriched in the obese host microbiome compared to the lean host microbiome. Table V represents the accession numbers of 869 biomolecules involved in carbohydrate import that are enriched in the obese host microbiome compared to the lean host microbiome.

Additionally, the biomolecule may be at least 70, 75, 80, 85, 90, or 95% homologous to a biomolecule derived from an accession number detailed above. In one embodiment, the biomolecule may be at least 80, 81, 82, 83, 84, 85, 86, 87, 88, or 89% homologous to a biomolecule derived from an accession number detailed above. In another embodiment, the biomolecule may be at least 90, 91, 92, 93, 94, 95, 96, 97, 98, or 99% homologous to a biomolecule derived from an accession number detailed above.

In determining whether a biomolecule is substantially homologous or shares a certain percentage of sequence identity with a sequence of the invention, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See http://www.ncbi.nlm.nih.gov for more details.

For each of the above embodiments, methods of determining biomolecules that are indicative or, or modulated in, an obese host microbiome compared to a lean host microbiome may be determined using methods detailed in the Examples.

TABLE Z
Starch and sucrose metabolism

| | | | |
|---|---|---|---|
| AATA01000367.1 | AATA01000378.1 | AATA01000604.1 | AATA01000619.1 |
| AATA01000626.1 | AATA01000812.1 | AATA01000861.1 | AATA01001081.1 |
| AATA01001162.1 | AATA01001279.1 | AATA01001315.1 | AATA01001352.1 |
| AATA01001552.1 | AATA01001626.1 | AATA01001645.1 | AATA01001835.1 |
| AATA01001927.1 | AATA01002235.1 | AATA01002243.1 | AATA01002245.1 |
| AATA01002354.1 | AATA01002406.1 | AATA01002523.1 | AATA01002663.1 |
| AATA01002708.1 | AATA01002712.1 | AATA01002826.1 | AATA01002865.1 |
| AATA01002884.1 | AATA01002939.1 | AATA01002955.1 | AATA01002994.1 |
| AATA01003014.1 | AATA01003144.1 | AATA01003220.1 | AATA01003314.1 |
| AATA01003592.1 | AATA01003657.1 | AATA01003741.1 | AATA01003877.1 |
| AATA01004137.1 | AATA01004174.1 | AATA01004366.1 | AATA01004387.1 |
| AATA01004465.1 | AATA01004518.1 | AATA01004607.1 | AATA01004681.1 |
| AATA01004688.1 | AATA01004723.1 | AATA01004736.1 | AATA01004871.1 |
| AATA01004904.1 | AATA01004932.1 | AATA01005085.1 | AATA01005122.1 |
| AATA01005201.1 | AATA01005300.1 | AATA01005319.1 | AATA01005501.1 |
| AATA01005538.1 | AATA01005692.1 | AATA01005728.1 | AATA01006002.1 |
| AATA01006129.1 | AATA01006149.1 | AATA01006278.1 | AATA01006286.1 |
| AATA01006335.1 | AATA01006431.1 | AATA01006513.1 | AATA01006879.1 |
| AATA01006985.1 | AATA01007186.1 | AATA01007348.1 | AATA01007637.1 |
| AATA01007781.1 | AATA01008402.1 | AATA01008580.1 | AATA01008670.1 |
| AATA01008918.1 | AATA01009070.1 | AATA01009141.1 | AATA01009144.1 |
| AATA01009153.1 | AATA01009379.1 | AATA01009421.1 | AATA01009439.1 |
| AATA01009527.1 | AATA01009635.1 | AATA01009673.1 | AATA01009760.1 |
| AATA01009879.1 | AATA01010032.1 | AATA01010127.1 | AATA01010306.1 |
| AATA01010423.1 | AATB01000541.1 | AATB01000828.1 | AATB01000866.1 |
| AATB01001143.1 | AATB01001302.1 | AATB01001307.1 | AATB01001311.1 |
| AATB01001359.1 | AATB01001422.1 | AATB01001587.1 | AATB01001641.1 |
| AATB01001707.1 | AATB01001953.1 | AATB01001986.1 | AATB01001991.1 |
| AATB01002005.1 | AATB01002085.1 | AATB01002213.1 | AATB01002368.1 |
| AATB01002391.1 | AATB01002599.1 | AATB01002717.1 | AATB01002889.1 |
| AATB01002892.1 | AATB01003163.1 | AATB01003217.1 | AATB01003396.1 |
| AATB01003515.1 | AATB01003596.1 | AATB01003671.1 | AATB01003715.1 |
| AATB01003838.1 | AATB01003979.1 | AATB01004028.1 | AATB01004958.1 |
| AATB01005356.1 | AATB01006000.1 | AATB01006136.1 | AATB01006159.1 |
| AATB01006233.1 | AATB01006389.1 | AATB01006647.1 | AATB01006754.1 |
| AATB01006908.1 | AATB01006919.1 | AATB01006926.1 | AATB01006935.1 |
| AATB01007750.1 | AATB01007943.1 | AATB01008155.1 | AATB01008432.1 |
| AATB01008453.1 | AATB01008635.1 | AATB01008636.1 | AATB01009265.1 |
| AATB01009430.1 | AATB01009668.1 | AATB01009708.1 | AATB01009907.1 |
| AATB01009949.1 | AATB01010152.1 | AATB01010429.1 | AATB01010485.1 |
| AATB01010566.1 | AATB01010591.1 | AATB01010614.1 | AATB01010703.1 |
| AATB01011114.1 | AATB01011135.1 | AATC01000258.1 | AATC01000287.1 |
| AATC01000304.1 | AATC01000371.1 | AATC01000530.1 | AATC01000565.1 |
| AATC01000603.1 | AATC01000608.1 | AATC01000684.1 | AATC01000687.1 |
| AATC01000731.1 | AATC01000774.1 | AATC01000842.1 | AATC01000892.1 |
| AATC01000911.1 | AATC01000998.1 | AATC01001054.1 | AATC01001100.1 |
| AATC01001151.1 | AATC01001177.1 | AATC01001184.1 | AATC01001227.1 |
| AATC01001267.1 | AATC01001350.1 | AATC01001408.1 | AATC01001426.1 |
| AATC01001459.1 | AATC01001480.1 | AATC01001552.1 | AATC01001685.1 |
| AATC01001711.1 | AATC01001747.1 | AATC01001759.1 | AATC01001846.1 |
| AATC01001990.1 | AATC01002000.1 | AATC01002009.1 | AATC01002024.1 |
| AATC01002090.1 | AATC01002127.1 | AATC01002172.1 | AATC01002210.1 |
| AATC01002299.1 | AATC01002329.1 | AATC01002344.1 | AATC01002357.1 |
| AATC01002445.1 | AATC01002465.1 | AATC01002560.1 | AATC01002578.1 |
| AATC01002607.1 | AATC01002624.1 | AATC01002648.1 | AATC01002727.1 |
| AATC01002866.1 | AATC01002879.1 | AATC01002942.1 | AATC01002955.1 |
| AATC01002963.1 | AATC01003024.1 | AATC01003029.1 | AATC01003130.1 |
| AATC01003154.1 | AATC01003160.1 | AATC01003195.1 | AATC01003200.1 |
| AATC01003225.1 | AATC01003262.1 | AATC01003382.1 | AATC01003392.1 |
| AATC01003402.1 | AATC01003434.1 | AATC01003436.1 | AATC01003568.1 |
| AATC01003650.1 | AATC01003652.1 | AATC01003716.1 | AATC01003871.1 |
| AATC01003874.1 | AATC01003891.1 | AATC01003916.1 | AATC01003933.1 |
| AATC01003992.1 | AATC01004072.1 | AATC01004086.1 | AATC01004275.1 |
| AATC01004294.1 | AATC01004330.1 | AATC01004346.1 | AATC01004392.1 |
| AATC01004398.1 | AATC01004407.1 | AATC01004442.1 | AATC01004563.1 |
| AATC01004594.1 | AATC01004622.1 | AATC01004683.1 | AATC01004784.1 |
| AATC01004844.1 | AATC01004885.1 | AATC01004949.1 | AATC01004952.1 |
| AATC01004959.1 | AATC01004979.1 | AATC01004992.1 | AATC01005038.1 |
| AATC01005060.1 | AATC01005067.1 | AATC01005139.1 | AATC01005150.1 |
| AATC01005255.1 | AATC01005305.1 | AATC01005366.1 | AATC01005548.1 |
| AATC01005596.1 | AATC01005667.1 | AATC01005710.1 | AATC01005725.1 |
| AATC01005781.1 | AATC01005791.1 | AATC01005825.1 | AATC01005892.1 |
| AATC01005918.1 | AATC01005994.1 | AATC01006282.1 | AATC01006321.1 |
| AATC01006345.1 | AATC01006348.1 | AATC01006547.1 | AATC01006642.1 |
| AATC01006644.1 | AATC01006710.1 | AATC01006770.1 | AATC01006788.1 |
| AATC01006794.1 | AATC01006798.1 | AATC01006801.1 | AATC01006817.1 |
| AATC01006895.1 | AATC01006950.1 | AATC01006976.1 | AATC01007020.1 |
| AATC01007041.1 | AATC01007109.1 | AATC01007158.1 | AATC01007175.1 |
| AATC01007207.1 | AATC01007233.1 | AATC01007273.1 | AATC01007507.1 |
| AATC01007551.1 | AATC01007571.1 | AATC01007583.1 | AATC01007608.1 |
| AATC01007644.1 | AATC01007696.1 | AATC01007715.1 | AATC01007756.1 |
| AATC01007782.1 | AATC01007812.1 | AATC01007882.1 | AATC01007944.1 |
| AATC01008034.1 | AATC01008188.1 | AATC01008195.1 | AATC01008305.1 |
| AATC01008420.1 | AATC01008484.1 | AATC01008559.1 | AATC01008756.1 |
| AATC01008968.1 | AATC01008973.1 | AATC01009076.1 | AATC01009132.1 |
| AATC01009148.1 | AATC01009210.1 | AATD01000417.1 | AATD01000450.1 |
| AATD01000483.1 | AATD01000495.1 | AATD01000578.1 | AATD01000590.1 |
| AATD01000592.1 | AATD01000617.1 | AATD01000667.1 | AATD01000692.1 |
| AATD01000700.1 | AATD01000701.1 | AATD01000817.1 | AATD01000870.1 |
| AATD01000895.1 | AATD01001162.1 | AATD01001216.1 | AATD01001248.1 |
| AATD01001259.1 | AATD01001305.1 | AATD01001317.1 | AATD01001322.1 |
| AATD01001360.1 | AATD01001400.1 | AATD01001534.1 | AATD01001567.1 |
| AATD01001580.1 | AATD01001592.1 | AATD01001653.1 | AATD01001690.1 |
| AATD01001755.1 | AATD01001774.1 | AATD01001896.1 | AATD01001902.1 |
| AATD01001918.1 | AATD01001922.1 | AATD01001948.1 | AATD01001974.1 |
| AATD01002028.1 | AATD01002041.1 | AATD01002051.1 | AATD01002095.1 |
| AATD01002097.1 | AATD01002126.1 | AATD01002165.1 | AATD01002168.1 |
| AATD01002169.1 | AATD01002186.1 | AATD01002395.1 | AATD01002453.1 |
| AATD01002472.1 | AATD01002548.1 | AATD01002596.1 | AATD01002739.1 |
| AATD01002778.1 | AATD01002817.1 | AATD01002964.1 | AATD01003003.1 |
| AATD01003182.1 | AATD01003199.1 | AATD01003222.1 | AATD01003264.1 |
| AATD01003296.1 | AATD01003384.1 | AATD01003453.1 | AATD01003557.1 |
| AATD01003608.1 | AATD01003759.1 | AATD01003803.1 | AATD01003884.1 |
| AATD01004067.1 | AATD01004083.1 | AATD01004161.1 | AATD01004184.1 |
| AATD01004186.1 | AATD01004300.1 | AATD01004313.1 | AATD01004319.1 |
| AATD01004475.1 | AATD01004607.1 | AATD01004618.1 | AATD01004644.1 |
| AATD01004760.1 | AATD01004779.1 | AATD01004788.1 | AATD01004797.1 |
| AATD01004923.1 | AATD01004935.1 | AATD01004970.1 | AATD01005048.1 |
| AATD01005176.1 | AATD01005198.1 | AATD01005260.1 | AATD01005276.1 |
| AATD01005402.1 | AATD01005457.1 | AATD01005559.1 | AATD01005574.1 |
| AATD01005580.1 | AATD01005613.1 | AATD01005675.1 | AATD01005694.1 |
| AATD01005742.1 | AATD01005743.1 | AATD01005837.1 | AATD01005915.1 |
| AATD01005919.1 | AATD01005940.1 | AATD01005958.1 | AATD01005992.1 |
| AATD01006063.1 | AATD01006088.1 | AATD01006123.1 | AATD01006191.1 |
| AATD01006205.1 | AATD01006240.1 | AATD01006275.1 | AATD01006409.1 |
| AATD01006524.1 | AATD01006610.1 | AATD01006638.1 | AATD01006719.1 |
| AATD01006732.1 | AATD01006783.1 | AATD01007055.1 | AATD01007082.1 |
| AATD01007119.1 | AATD01007171.1 | AATD01007291.1 | AATD01007301.1 |
| AATD01007386.1 | AATD01007431.1 | AATD01007525.1 | AATD01007572.1 |
| AATD01007645.1 | AATD01007670.1 | AATD01007680.1 | AATD01007739.1 |
| AATD01007740.1 | AATD01007760.1 | AATD01007763.1 | AATD01007884.1 |
| AATD01007984.1 | AATD01008070.1 | AATD01008133.1 | AATD01008140.1 |
| AATD01008333.1 | AATD01008354.1 | AATD01008358.1 | AATD01008447.1 |
| AATD01008482.1 | AATD01008755.1 | AATD01008814.1 | AATD01008829.1 |
| AATD01008904.1 | AATD01008967.1 | AATD01009012.1 | AATD01009079.1 |
| AATD01009091.1 | AATD01009209.1 | AATD01009218.1 | AATD01009406.1 |
| AATD01009708.1 | AATD01009803.1 | AATD01009887.1 | AATD01010045.1 |
| AATD01010117.1 | AATD01010291.1 | AATD01010417.1 | AATE01000308.1 |
| AATE01000370.1 | AATE01000448.1 | AATE01000480.1 | AATE01000499.1 |
| AATE01000507.1 | AATE01000582.1 | AATE01000587.1 | AATE01000694.1 |
| AATE01000769.1 | AATE01000944.1 | AATE01001080.1 | AATE01001116.1 |
| AATE01001133.1 | AATE01001191.1 | AATE01001255.1 | AATE01001284.1 |
| AATE01001287.1 | AATE01001291.1 | AATE01001296.1 | AATE01001322.1 |
| AATE01001391.1 | AATE01001410.1 | AATE01001429.1 | AATE01001447.1 |
| AATE01001485.1 | AATE01001571.1 | AATE01001605.1 | AATE01001726.1 |
| AATE01001837.1 | AATE01001916.1 | AATE01002002.1 | AATE01002010.1 |
| AATE01002054.1 | AATE01002129.1 | AATE01002478.1 | AATE01002491.1 |
| AATE01002639.1 | AATE01002642.1 | AATE01002752.1 | AATE01002805.1 |
| AATE01002827.1 | AATE01002876.1 | AATE01002910.1 | AATE01002927.1 |
| AATE01002930.1 | AATE01002966.1 | AATE01003068.1 | AATE01003115.1 |
| AATE01003117.1 | AATE01003209.1 | AATE01003321.1 | AATE01003471.1 |
| AATE01003513.1 | AATE01003545.1 | AATE01003606.1 | AATE01003640.1 |
| AATE01003711.1 | AATE01003753.1 | AATE01003797.1 | AATE01003918.1 |
| AATE01003988.1 | AATE01004230.1 | AATE01004265.1 | AATE01004275.1 |
| AATE01004341.1 | AATE01004344.1 | AATE01004359.1 | AATE01004397.1 |
| AATE01004780.1 | AATE01004806.1 | AATE01004832.1 | AATE01004848.1 |
| AATE01004874.1 | AATE01005032.1 | AATE01005110.1 | AATE01005223.1 |
| AATE01005284.1 | AATE01005347.1 | AATE01005425.1 | AATE01005430.1 |
| AATE01005453.1 | AATE01005503.1 | AATE01005516.1 | AATE01005628.1 |
| AATE01005751.1 | AATE01005984.1 | AATE01005987.1 | AATE01005997.1 |
| AATE01006310.1 | AATE01006483.1 | AATE01006505.1 | AATE01006523.1 |
| AATE01006684.1 | AATE01006715.1 | AATE01006774.1 | AATE01006838.1 |
| AATE01006921.1 | AATE01006965.1 | AATE01006992.1 | AATE01007020.1 |
| AATE01007104.1 | AATE01007332.1 | AATE01007446.1 | AATE01007477.1 |
| AATE01007487.1 | AATE01007572.1 | AATE01007581.1 | AATE01007637.1 |
| AATE01007670.1 | AATE01007813.1 | AATE01007853.1 | AATE01007863.1 |
| AATE01007865.1 | AATE01008009.1 | AATE01008113.1 | AATE01008332.1 |
| AATE01008416.1 | | | |

TABLE Y
Galactose metabolism

| | | | |
|---|---|---|---|
| AATA01000364.1 | AATA01001208.1 | AATA01001269.1 | AATA01001302.1 |
| AATA01001530.1 | AATA01001794.1 | AATA01001880.1 | AATA01001927.1 |
| AATA01001998.1 | AATA01002782.1 | AATA01002826.1 | AATA01002838.1 |
| AATA01002927.1 | AATA01003314.1 | AATA01003511.1 | AATA01003657.1 |
| AATA01004057.1 | AATA01004156.1 | AATA01004179.1 | AATA01004301.1 |
| AATA01004387.1 | AATA01004448.1 | AATA01004634.1 | AATA01004643.1 |
| AATA01004657.1 | AATA01004683.1 | AATA01005518.1 | AATA01005535.1 |
| AATA01006014.1 | AATA01006041.1 | AATA01006173.1 | AATA01006335.1 |
| AATA01006349.1 | AATA01006704.1 | AATA01007290.1 | AATA01007352.1 |
| AATA01007470.1 | AATA01007717.1 | AATA01008261.1 | AATA01008580.1 |
| AATA01008582.1 | AATA01008670.1 | AATA01008996.1 | AATA01009120.1 |
| AATA01009155.1 | AATA01009419.1 | AATA01009690.1 | AATA01009869.1 |
| AATA01009914.1 | AATA01009942.1 | AATA01010032.1 | AATA01010051.1 |
| AATB01000866.1 | AATB01000983.1 | AATB01001125.1 | AATB01002005.1 |
| AATB01002085.1 | AATB01002128.1 | AATB01002512.1 | AATB01002942.1 |
| AATB01003728.1 | AATB01004292.1 | AATB01004589.1 | AATB01004893.1 |
| AATB01005776.1 | AATB01005876.1 | AATB01006159.1 | AATB01006233.1 |
| AATB01006707.1 | AATB01006981.1 | AATB01007416.1 | AATB01007666.1 |
| AATB01008155.1 | AATB01008668.1 | AATB01009265.1 | AATB01009587.1 |
| AATB01009693.1 | AATB01009765.1 | AATB01010238.1 | AATB01010566.1 |
| AATB01010624.1 | AATC01000464.1 | AATC01000511.1 | AATC01000579.1 |
| AATC01000949.1 | AATC01001846.1 | AATC01001944.1 | AATC01002423.1 |
| AATC01002942.1 | AATC01003054.1 | AATC01003114.1 | AATC01003382.1 |
| AATC01003568.1 | AATC01003750.1 | AATC01004209.1 | AATC01005013.1 |
| AATC01005150.1 | AATC01005251.1 | AATC01005327.1 | AATC01005335.1 |
| AATC01005489.1 | AATC01005624.1 | AATC01005791.1 | AATC01005825.1 |
| AATC01005918.1 | AATC01005978.1 | AATC01006168.1 | AATC01006305.1 |
| AATC01006895.1 | AATC01007014.1 | AATC01007273.1 | AATC01007447.1 |
| AATC01007620.1 | AATC01007699.1 | AATC01007715.1 | AATC01007759.1 |
| AATC01007944.1 | AATC01008188.1 | AATC01008273.1 | AATC01009076.1 |
| AATC01009132.1 | AATC01009381.1 | AATC01009482.1 | AATC01009752.1 |
| AATD01000574.1 | AATD01000948.1 | AATD01000982.1 | AATD01001338.1 |
| AATD01001342.1 | AATD01001360.1 | AATD01001567.1 | AATD01002333.1 |
| AATD01002469.1 | AATD01002969.1 | AATD01003167.1 | AATD01003676.1 |
| AATD01003784.1 | AATD01003919.1 | AATD01004004.1 | AATD01004357.1 |
| AATD01004715.1 | AATD01004791.1 | AATD01004845.1 | AATD01004887.1 |
| AATD01005117.1 | AATD01005494.1 | AATD01005874.1 | AATD01006550.1 |
| AATD01006577.1 | AATD01006585.1 | AATD01007211.1 | AATD01007618.1 |
| AATD01007837.1 | AATD01007984.1 | AATD01008181.1 | AATD01008191.1 |
| AATD01008355.1 | AATD01008641.1 | AATD01008755.1 | AATD01009058.1 |
| AATD01009102.1 | AATD01009377.1 | AATD01009406.1 | AATD01009509.1 |
| AATD01009708.1 | AATD01010045.1 | AATD01010150.1 | AATD01010417.1 |
| AATE01000573.1 | AATE01000685.1 | AATE01000743.1 | AATE01001204.1 |
| AATE01001517.1 | AATE01001588.1 | AATE01001661.1 | AATE01001729.1 |
| AATE01001735.1 | AATE01001837.1 | AATE01001859.1 | AATE01001929.1 |
| AATE01001932.1 | AATE01002180.1 | AATE01002491.1 | AATE01002500.1 |
| AATE01002777.1 | AATE01002846.1 | AATE01003919.1 | AATE01004109.1 |
| AATE01004230.1 | AATE01004342.1 | AATE01004487.1 | AATE01004600.1 |
| AATE01004792.1 | AATE01004793.1 | AATE01005455.1 | AATE01005465.1 |
| AATE01005628.1 | AATE01005987.1 | AATE01006089.1 | AATE01006333.1 |
| AATE01006472.1 | AATE01007195.1 | AATE01007261.1 | AATE01007301.1 |
| AATE01007912.1 | | | |

TABLE X
Butanoate metabolism

| | | | |
|---|---|---|---|
| AATA01000644.1 | AATA01001167.1 | AATA01001250.1 | AATA01002159.1 |
| AATA01002922.1 | AATA01003720.1 | AATA01003830.1 | AATA01004132.1 |
| AATA01004146.1 | AATA01004278.1 | AATA01004287.1 | AATA01004779.1 |
| AATA01005204.1 | AATA01005614.1 | AATA01006915.1 | AATA01008164.1 |
| AATA01009218.1 | AATA01009505.1 | AATA01009533.1 | AATA01009725.1 |
| AATA01010088.1 | AATA01010256.1 | AATB01000530.1 | AATB01000821.1 |
| AATB01003115.1 | AATB01003466.1 | AATB01003612.1 | AATB01003692.1 |
| AATB01003748.1 | AATB01004113.1 | AATB01005179.1 | AATB01005626.1 |
| AATB01006406.1 | AATB01007003.1 | AATB01007143.1 | AATB01007347.1 |
| AATB01007536.1 | AATB01007719.1 | AATB01009516.1 | AATB01010198.1 |
| AATB01010413.1 | AATB01010772.1 | AATC01000930.1 | AATC01001211.1 |
| AATC01001417.1 | AATC01001542.1 | AATC01001583.1 | AATC01001785.1 |
| AATC01002540.1 | AATC01003252.1 | AATC01003508.1 | AATC01003890.1 |
| AATC01004159.1 | AATC01004206.1 | AATC01004856.1 | AATC01005074.1 |
| AATC01005740.1 | AATC01006325.1 | AATC01006593.1 | AATC01006610.1 |
| AATC01007057.1 | AATC01007281.1 | AATC01007335.1 | AATC01007438.1 |
| AATC01007575.1 | AATC01007815.1 | AATC01008204.1 | AATC01008348.1 |
| AATC01008417.1 | AATC01008488.1 | AATC01008728.1 | AATC01009201.1 |
| AATC01009321.1 | AATC01009428.1 | AATD01000560.1 | AATD01001080.1 |
| AATD01001118.1 | AATD01001861.1 | AATD01002172.1 | AATD01002433.1 |
| AATD01002839.1 | AATD01003082.1 | AATD01003422.1 | AATD01003433.1 |
| AATD01003491.1 | AATD01004323.1 | AATD01006189.1 | AATD01007344.1 |
| AATD01007960.1 | AATD01007980.1 | AATD01007981.1 | AATD01008180.1 |
| AATD01008255.1 | AATD01008286.1 | AATD01009476.1 | AATD01009477.1 |
| AATD01009533.1 | AATD01009692.1 | AATD01009926.1 | AATD01009946.1 |
| AATD01010353.1 | AATE01000586.1 | AATE01001750.1 | AATE01002442.1 |
| AATE01002516.1 | AATE01002768.1 | AATE01002862.1 | AATE01003029.1 |
| AATE01003071.1 | AATE01003308.1 | AATE01003617.1 | AATE01003652.1 |
| AATE01004436.1 | AATE01004528.1 | AATE01004575.1 | AATE01005584.1 |
| AATE01005838.1 | AATE01006057.1 | AATE01006232.1 | AATE01006597.1 |
| AATE01007131.1 | AATE01007812.1 | AATE01007939.1 | AATE01008055.1 |

TABLE W
Acetogenesis

| | | | |
|---|---|---|---|
| AATA01001505.1 | AATA01004859.1 | AATB01006849.1 | AATC01008727.1 |
| AATC01009120.1 | AATD01002372.1 | AATD01008617.1 | AATD01008638.1 |
| AATD01010214.1 | AATD01010397.1 | AATE01002545.1 | AATE01003350.1 |
| AATE01006283.1 | AATE01006955.1 | | |

TABLE V
Carbohydrate import

| | | | |
|---|---|---|---|
| AATA01000244.1 | AATA01000256.1 | AATA01000264.1 | AATA01000407.1 |
| AATA01000454.1 | AATA01000460.1 | AATA01000649.1 | AATA01000657.1 |
| AATA01000704.1 | AATA01000718.1 | AATA01000840.1 | AATA01000914.1 |
| AATA01000918.1 | AATA01000924.1 | AATA01000941.1 | AATA01000948.1 |
| AATA01001055.1 | AATA01001105.1 | AATA01001107.1 | AATA01001118.1 |
| AATA01001169.1 | AATA01001184.1 | AATA01001211.1 | AATA01001240.1 |
| AATA01001350.1 | AATA01001402.1 | AATA01001449.1 | AATA01001468.1 |
| AATA01001519.1 | AATA01001548.1 | AATA01001596.1 | AATA01001674.1 |
| AATA01001683.1 | AATA01001756.1 | AATA01001798.1 | AATA01001826.1 |
| AATA01001960.1 | AATA01001988.1 | AATA01002023.1 | AATA01002061.1 |
| AATA01002091.1 | AATA01002097.1 | AATA01002127.1 | AATA01002286.1 |
| AATA01002295.1 | AATA01002302.1 | AATA01002314.1 | AATA01002315.1 |
| AATA01002485.1 | AATA01002533.1 | AATA01002571.1 | AATA01002592.1 |
| AATA01002623.1 | AATA01002702.1 | AATA01002745.1 | AATA01002806.1 |
| AATA01002830.1 | AATA01003217.1 | AATA01003262.1 | AATA01003344.1 |
| AATA01003372.1 | AATA01003398.1 | AATA01003463.1 | AATA01003589.1 |
| AATA01003600.1 | AATA01003629.1 | AATA01003690.1 | AATA01003817.1 |
| AATA01003835.1 | AATA01003903.1 | AATA01003919.1 | AATA01003978.1 |
| AATA01003984.1 | AATA01004149.1 | AATA01004209.1 | AATA01004273.1 |
| AATA01004289.1 | AATA01004378.1 | AATA01004450.1 | AATA01004524.1 |
| AATA01004807.1 | AATA01004870.1 | AATA01004892.1 | AATA01004901.1 |
| AATA01004924.1 | AATA01005026.1 | AATA01005173.1 | AATA01005175.1 |
| AATA01005188.1 | AATA01005375.1 | AATA01005474.1 | AATA01005476.1 |
| AATA01005513.1 | AATA01005542.1 | AATA01005605.1 | AATA01005621.1 |
| AATA01005635.1 | AATA01005718.1 | AATA01005737.1 | AATA01005795.1 |
| AATA01005832.1 | AATA01005849.1 | AATA01005865.1 | AATA01006008.1 |
| AATA01006060.1 | AATA01006125.1 | AATA01006136.1 | AATA01006198.1 |
| AATA01006210.1 | AATA01006289.1 | AATA01006308.1 | AATA01006357.1 |
| AATA01006404.1 | AATA01006447.1 | AATA01006466.1 | AATA01006496.1 |
| AATA01006517.1 | AATA01006537.1 | AATA01006561.1 | AATA01006573.1 |
| AATA01006591.1 | AATA01006676.1 | AATA01006731.1 | AATA01006792.1 |
| AATA01006823.1 | AATA01006839.1 | AATA01006863.1 | AATA01006887.1 |
| AATA01006917.1 | AATA01006940.1 | AATA01006964.1 | AATA01007124.1 |
| AATA01007141.1 | AATA01007312.1 | AATA01007314.1 | AATA01007357.1 |
| AATA01007369.1 | AATA01007395.1 | AATA01007422.1 | AATA01007430.1 |
| AATA01007488.1 | AATA01007553.1 | AATA01007571.1 | AATA01007610.1 |
| AATA01007648.1 | AATA01007651.1 | AATA01007792.1 | AATA01007869.1 |
| AATA01007892.1 | AATA01007900.1 | AATA01008013.1 | AATA01008049.1 |
| AATA01008168.1 | AATA01008203.1 | AATA01008257.1 | AATA01008283.1 |
| AATA01008296.1 | AATA01008314.1 | AATA01008323.1 | AATA01008515.1 |
| AATA01008573.1 | AATA01008611.1 | AATA01008856.1 | AATA01008860.1 |
| AATA01008926.1 | AATA01009002.1 | AATA01009048.1 | AATA01009079.1 |
| AATA01009208.1 | AATA01009214.1 | AATA01009245.1 | AATA01009367.1 |
| AATA01009381.1 | AATA01009437.1 | AATA01009778.1 | AATA01009988.1 |
| AATA01010002.1 | AATA01010010.1 | AATA01010140.1 | AATA01010160.1 |
| AATA01010161.1 | AATA01010246.1 | AATA01010254.1 | AATA01010284.1 |
| AATA01010321.1 | AATA01010426.1 | AATA01010491.1 | AATB01000575.1 |
| AATB01000581.1 | AATB01000602.1 | AATB01000722.1 | AATB01000851.1 |
| AATB01000872.1 | AATB01000880.1 | AATB01000886.1 | AATB01000919.1 |
| AATB01000970.1 | AATB01001009.1 | AATB01001158.1 | AATB01001176.1 |
| AATB01001186.1 | AATB01001343.1 | AATB01001385.1 | AATB01001522.1 |
| AATB01001564.1 | AATB01001581.1 | AATB01001630.1 | AATB01001748.1 |
| AATB01001765.1 | AATB01001951.1 | AATB01001983.1 | AATB01002020.1 |
| AATB01002029.1 | AATB01002033.1 | AATB01002052.1 | AATB01002107.1 |
| AATB01002121.1 | AATB01002216.1 | AATB01002266.1 | AATB01002400.1 |
| AATB01002415.1 | AATB01002469.1 | AATB01002485.1 | AATB01002514.1 |
| AATB01002749.1 | AATB01003053.1 | AATB01003184.1 | AATB01003196.1 |
| AATB01003215.1 | AATB01003233.1 | AATB01003278.1 | AATB01003643.1 |
| AATB01003900.1 | AATB01003909.1 | AATB01004000.1 | AATB01004086.1 |
| AATB01004172.1 | AATB01004341.1 | AATB01004438.1 | AATB01004470.1 |
| AATB01004487.1 | AATB01004670.1 | AATB01004797.1 | AATB01004850.1 |
| AATB01004902.1 | AATB01005206.1 | AATB01005283.1 | AATB01005386.1 |
| AATB01005444.1 | AATB01005574.1 | AATB01005614.1 | AATB01005733.1 |
| AATB01005987.1 | AATB01006042.1 | AATB01006334.1 | AATB01006560.1 |
| AATB01006677.1 | AATB01006739.1 | AATB01006907.1 | AATB01007087.1 |
| AATB01007199.1 | AATB01007305.1 | AATB01007439.1 | AATB01007624.1 |
| AATB01007758.1 | AATB01007777.1 | AATB01007842.1 | AATB01008029.1 |
| AATB01008092.1 | AATB01008115.1 | AATB01008159.1 | AATB01008190.1 |
| AATB01008378.1 | AATB01008550.1 | AATB01008589.1 | AATB01008901.1 |
| AATB01008990.1 | AATB01009020.1 | AATB01009175.1 | AATB01009262.1 |
| AATB01009270.1 | AATB01009476.1 | AATB01009482.1 | AATB01009498.1 |
| AATB01009508.1 | AATB01009546.1 | AATB01009832.1 | AATB01009964.1 |
| AATB01010358.1 | AATB01010453.1 | AATB01010547.1 | AATB01010598.1 |
| AATB01010794.1 | AATB01010980.1 | AATB01011205.1 | AATC01000266.1 |
| AATC01000276.1 | AATC01000400.1 | AATC01000444.1 | AATC01000469.1 |
| AATC01000480.1 | AATC01000529.1 | AATC01000552.1 | AATC01000557.1 |
| AATC01000633.1 | AATC01000641.1 | AATC01000672.1 | AATC01000699.1 |
| AATC01000706.1 | AATC01000793.1 | AATC01000952.1 | AATC01001028.1 |
| AATC01001040.1 | AATC01001103.1 | AATC01001127.1 | AATC01001141.1 |
| AATC01001370.1 | AATC01001394.1 | AATC01001427.1 | AATC01001431.1 |
| AATC01001570.1 | AATC01001618.1 | AATC01001666.1 | AATC01001673.1 |
| AATC01001734.1 | AATC01001739.1 | AATC01001766.1 | AATC01001783.1 |
| AATC01001852.1 | AATC01001873.1 | AATC01001946.1 | AATC01001947.1 |
| AATC01002005.1 | AATC01002016.1 | AATC01002057.1 | AATC01002196.1 |
| AATC01002260.1 | AATC01002281.1 | AATC01002369.1 | AATC01002400.1 |
| AATC01002415.1 | AATC01002436.1 | AATC01002700.1 | AATC01002709.1 |
| AATC01002819.1 | AATC01002959.1 | AATC01003071.1 | AATC01003221.1 |
| AATC01003254.1 | AATC01003343.1 | AATC01003349.1 | AATC01003428.1 |
| AATC01003517.1 | AATC01003579.1 | AATC01003690.1 | AATC01003703.1 |
| AATC01003794.1 | AATC01003818.1 | AATC01003838.1 | AATC01003842.1 |
| AATC01003886.1 | AATC01004096.1 | AATC01004140.1 | AATC01004146.1 |
| AATC01004189.1 | AATC01004263.1 | AATC01004439.1 | AATC01004488.1 |
| AATC01004536.1 | AATC01004580.1 | AATC01004627.1 | AATC01004739.1 |
| AATC01004818.1 | AATC01005066.1 | AATC01005092.1 | AATC01005108.1 |
| AATC01005125.1 | AATC01005175.1 | AATC01005254.1 | AATC01005273.1 |
| AATC01005326.1 | AATC01005328.1 | AATC01005372.1 | AATC01005390.1 |
| AATC01005427.1 | AATC01005464.1 | AATC01005470.1 | AATC01005513.1 |
| AATC01005583.1 | AATC01005702.1 | AATC01005707.1 | AATC01005738.1 |
| AATC01005742.1 | AATC01005875.1 | AATC01005876.1 | AATC01006042.1 |
| AATC01006133.1 | AATC01006148.1 | AATC01006231.1 | AATC01006314.1 |
| AATC01006341.1 | AATC01006364.1 | AATC01006389.1 | AATC01006444.1 |
| AATC01006537.1 | AATC01006546.1 | AATC01006554.1 | AATC01006560.1 |
| AATC01006608.1 | AATC01006664.1 | AATC01006925.1 | AATC01007029.1 |
| AATC01007324.1 | AATC01007358.1 | AATC01007560.1 | AATC01007857.1 |
| AATC01007881.1 | AATC01008013.1 | AATC01008032.1 | AATC01008115.1 |
| AATC01008201.1 | AATC01008216.1 | AATC01008229.1 | AATC01008317.1 |
| AATC01008371.1 | AATC01008394.1 | AATC01008454.1 | AATC01008467.1 |
| AATC01008586.1 | AATC01008590.1 | AATC01008653.1 | AATC01008657.1 |
| AATC01008704.1 | AATC01008708.1 | AATC01008769.1 | AATC01008790.1 |
| AATC01008882.1 | AATC01009143.1 | AATC01009160.1 | AATC01009174.1 |
| AATC01009216.1 | AATC01009236.1 | AATC01009264.1 | AATC01009285.1 |
| AATC01009305.1 | AATC01009318.1 | AATC01009421.1 | AATC01009813.1 |
| AATC01009852.1 | AATC01010089.1 | AATC01010227.1 | AATD01000362.1 |
| AATD01000426.1 | AATD01000446.1 | AATD01000456.1 | AATD01000731.1 |
| AATD01000753.1 | AATD01000832.1 | AATD01000900.1 | AATD01000922.1 |
| AATD01001011.1 | AATD01001014.1 | AATD01001046.1 | AATD01001049.1 |
| AATD01001073.1 | AATD01001076.1 | AATD01001178.1 | AATD01001275.1 |
| AATD01001276.1 | AATD01001302.1 | AATD01001336.1 | AATD01001369.1 |
| AATD01001378.1 | AATD01001433.1 | AATD01001449.1 | AATD01001466.1 |
| AATD01001542.1 | AATD01001607.1 | AATD01001610.1 | AATD01001696.1 |
| AATD01001850.1 | AATD01001900.1 | AATD01001975.1 | AATD01001999.1 |
| AATD01002043.1 | AATD01002092.1 | AATD01002139.1 | AATD01002154.1 |
| AATD01002285.1 | AATD01002290.1 | AATD01002310.1 | AATD01002409.1 |
| AATD01002431.1 | AATD01002452.1 | AATD01002456.1 | AATD01002715.1 |
| AATD01002795.1 | AATD01002800.1 | AATD01002847.1 | AATD01002906.1 |
| AATD01002933.1 | AATD01002996.1 | AATD01003001.1 | AATD01003019.1 |
| AATD01003055.1 | AATD01003120.1 | AATD01003121.1 | AATD01003190.1 |
| AATD01003252.1 | AATD01003323.1 | AATD01003334.1 | AATD01003349.1 |
| AATD01003352.1 | AATD01003428.1 | AATD01003448.1 | AATD01003470.1 |
| AATD01003518.1 | AATD01003541.1 | AATD01003550.1 | AATD01003589.1 |
| AATD01003623.1 | AATD01003628.1 | AATD01003709.1 | AATD01003727.1 |
| AATD01003753.1 | AATD01003868.1 | AATD01003892.1 | AATD01003900.1 |
| AATD01003917.1 | AATD01004018.1 | AATD01004028.1 | AATD01004055.1 |
| AATD01004112.1 | AATD01004145.1 | AATD01004162.1 | AATD01004190.1 |
| AATD01004222.1 | AATD01004286.1 | AATD01004364.1 | AATD01004455.1 |
| AATD01004512.1 | AATD01004556.1 | AATD01004578.1 | AATD01004625.1 |
| AATD01004716.1 | AATD01004764.1 | AATD01004770.1 | AATD01004772.1 |
| AATD01004785.1 | AATD01004896.1 | AATD01004907.1 | AATD01004927.1 |
| AATD01004971.1 | AATD01004992.1 | AATD01004997.1 | AATD01005014.1 |
| AATD01005033.1 | AATD01005042.1 | AATD01005128.1 | AATD01005130.1 |
| AATD01005136.1 | AATD01005162.1 | AATD01005189.1 | AATD01005195.1 |
| AATD01005280.1 | AATD01005285.1 | AATD01005426.1 | AATD01005451.1 |
| AATD01005477.1 | AATD01005616.1 | AATD01005687.1 | AATD01005696.1 |
| AATD01005757.1 | AATD01005790.1 | AATD01005853.1 | AATD01005866.1 |
| AATD01005911.1 | AATD01005988.1 | AATD01006038.1 | AATD01006057.1 |
| AATD01006070.1 | AATD01006104.1 | AATD01006128.1 | AATD01006159.1 |
| AATD01006192.1 | AATD01006234.1 | AATD01006242.1 | AATD01006291.1 |
| AATD01006313.1 | AATD01006366.1 | AATD01006447.1 | AATD01006506.1 |
| AATD01006516.1 | AATD01006555.1 | AATD01006575.1 | AATD01006576.1 |
| AATD01006592.1 | AATD01006630.1 | AATD01006737.1 | AATD01006791.1 |
| AATD01006886.1 | AATD01006893.1 | AATD01006915.1 | AATD01006976.1 |
| AATD01007017.1 | AATD01007029.1 | AATD01007039.1 | AATD01007042.1 |
| AATD01007097.1 | AATD01007166.1 | AATD01007172.1 | AATD01007206.1 |
| AATD01007214.1 | AATD01007260.1 | AATD01007340.1 | AATD01007409.1 |
| AATD01007438.1 | AATD01007500.1 | AATD01007537.1 | AATD01007624.1 |
| AATD01007663.1 | AATD01007672.1 | AATD01007678.1 | AATD01007839.1 |
| AATD01007868.1 | AATD01007878.1 | AATD01007910.1 | AATD01007912.1 |
| AATD01007941.1 | AATD01007952.1 | AATD01007990.1 | AATD01008025.1 |
| AATD01008032.1 | AATD01008043.1 | AATD01008051.1 | AATD01008064.1 |
| AATD01008116.1 | AATD01008124.1 | AATD01008142.1 | AATD01008297.1 |
| AATD01008352.1 | AATD01008467.1 | AATD01008584.1 | AATD01008634.1 |
| AATD01008662.1 | AATD01008690.1 | AATD01008732.1 | AATD01008836.1 |
| AATD01008872.1 | AATD01008877.1 | AATD01008906.1 | AATD01008935.1 |
| AATD01008956.1 | AATD01008970.1 | AATD01008992.1 | AATD01009104.1 |
| AATD01009111.1 | AATD01009295.1 | AATD01009344.1 | AATD01009428.1 |
| AATD01009487.1 | AATD01009542.1 | AATD01009612.1 | AATD01009628.1 |
| AATD01009664.1 | AATD01009786.1 | AATD01009849.1 | AATD01009900.1 |
| AATD01010014.1 | AATD01010029.1 | AATD01010088.1 | AATD01010089.1 |
| AATD01010091.1 | AATD01010095.1 | AATD01010100.1 | AATD01010121.1 |
| AATD01010143.1 | AATD01010213.1 | AATD01010262.1 | AATD01010270.1 |
| AATD01010276.1 | AATD01010360.1 | AATD01010367.1 | AATD01010418.1 |
| AATD01010468.1 | AATD01010475.1 | AATD01010566.1 | AATD01010640.1 |
| AATD01010774.1 | AATE01000277.1 | AATE01000420.1 | AATE01000473.1 |
| AATE01000627.1 | AATE01000752.1 | AATE01000788.1 | AATE01000916.1 |
| AATE01000984.1 | AATE01001099.1 | AATE01001141.1 | AATE01001199.1 |
| AATE01001233.1 | AATE01001276.1 | AATE01001278.1 | AATE01001333.1 |
| AATE01001342.1 | AATE01001375.1 | AATE01001403.1 | AATE01001428.1 |
| AATE01001430.1 | AATE01001436.1 | AATE01001502.1 | AATE01001561.1 |
| AATE01001586.1 | AATE01001653.1 | AATE01001751.1 | AATE01001784.1 |
| AATE01001815.1 | AATE01001835.1 | AATE01001877.1 | AATE01001980.1 |
| AATE01001987.1 | AATE01001991.1 | AATE01001998.1 | AATE01002065.1 |
| AATE01002076.1 | AATE01002152.1 | AATE01002203.1 | AATE01002293.1 |
| AATE01002306.1 | AATE01002316.1 | AATE01002347.1 | AATE01002479.1 |
| AATE01002485.1 | AATE01002544.1 | AATE01002590.1 | AATE01002601.1 |
| AATE01002619.1 | AATE01002673.1 | AATE01002675.1 | AATE01002684.1 |
| AATE01002759.1 | AATE01002761.1 | AATE01002811.1 | AATE01002825.1 |
| AATE01002858.1 | AATE01002884.1 | AATE01002957.1 | AATE01002972.1 |
| AATE01002983.1 | AATE01003026.1 | AATE01003061.1 | AATE01003082.1 |
| AATE01003088.1 | AATE01003090.1 | AATE01003119.1 | AATE01003263.1 |
| AATE01003272.1 | AATE01003373.1 | AATE01003532.1 | AATE01003636.1 |
| AATE01003697.1 | AATE01003771.1 | AATE01003776.1 | AATE01003832.1 |
| AATE01003904.1 | AATE01003955.1 | AATE01003983.1 | AATE01003997.1 |
| AATE01004030.1 | AATE01004043.1 | AATE01004165.1 | AATE01004247.1 |
| AATE01004386.1 | AATE01004429.1 | AATE01004456.1 | AATE01004541.1 |
| AATE01004547.1 | AATE01004631.1 | AATE01004632.1 | AATE01004635.1 |
| AATE01004637.1 | AATE01004724.1 | AATE01004730.1 | AATE01004777.1 |
| AATE01004782.1 | AATE01004787.1 | AATE01004843.1 | AATE01004903.1 |
| AATE01004996.1 | AATE01005006.1 | AATE01005068.1 | AATE01005107.1 |
| AATE01005137.1 | AATE01005161.1 | AATE01005167.1 | AATE01005194.1 |
| AATE01005360.1 | AATE01005384.1 | AATE01005435.1 | AATE01005475.1 |
| AATE01005494.1 | AATE01005531.1 | AATE01005534.1 | AATE01005535.1 |
| AATE01005545.1 | AATE01005551.1 | AATE01005553.1 | AATE01005587.1 |
| AATE01005740.1 | AATE01005745.1 | AATE01005772.1 | AATE01005803.1 |
| AATE01005809.1 | AATE01005825.1 | AATE01005846.1 | AATE01005932.1 |
| AATE01006043.1 | AATE01006055.1 | AATE01006069.1 | AATE01006132.1 |
| AATE01006136.1 | AATE01006228.1 | AATE01006267.1 | AATE01006388.1 |
| AATE01006427.1 | AATE01006431.1 | AATE01006437.1 | AATE01006598.1 |
| AATE01006680.1 | AATE01006688.1 | AATE01006704.1 | AATE01006747.1 |
| AATE01006764.1 | AATE01006815.1 | AATE01006816.1 | AATE01006825.1 |
| AATE01006907.1 | AATE01006979.1 | AATE01007012.1 | AATE01007015.1 |
| AATE01007105.1 | AATE01007112.1 | AATE01007130.1 | AATE01007217.1 |
| AATE01007231.1 | AATE01007259.1 | AATE01007269.1 | AATE01007425.1 |
| AATE01007483.1 | AATE01007502.1 | AATE01007645.1 | AATE01007707.1 |
| AATE01007824.1 | AATE01007840.1 | AATE01007884.1 | AATE01007930.1 |
| AATE01007962.1 | AATE01007986.1 | AATE01008011.1 | AATE01008025.1 |
| AATE01008138.1 | AATE01008149.1 | AATE01008205.1 | AATE01008212.1 |
| AATE01008368.1 | AATE01008403.1 | AATE01008413.1 | AATE01008415.1 |
| AATE01008506.1 | | | |

The arrays may be utilized in several suitable applications. For example, the arrays may be used in methods for detecting association between two or more biomolecules. This method typically comprises incubating a sample with the array under conditions such that the biomolecules comprising the sample may associate with the biomolecules attached to the array. The association is then detected, using means commonly known in the art, such as fluorescence. “Association,” as used in this context, may refer to hybridization, covalent binding, or ionic binding. A skilled artisan will appreciate that conditions under which association may occur will vary depending on the biomolecules, the substrate, and the detection method utilized. As such, suitable conditions may have to be optimized for each individual array created.

In yet another embodiment, the array may be used as a tool in a method to determine whether a compound has efficacy for treatment of obesity or an obesity-related disorder in a host. Alternatively, the array may be used as a tool in a method to determine whether a compound increases or decreases the relative abundance of Bacteriodes or Firmicutes in a subject. Typically, such methods comprise comparing a plurality of biomolecules of the host's microbiome before and after administration of a compound, such that if the abundance of biomolecules associated with obesity decreased after treatment, or the abundance of biomolecules indicative of Bacteroides increases, or the abundance of biomolecules indicative of Firmicutes decreases, the compound may be efficacious in treating obesity in a host.

The array may also be used to quantitate the plurality of biomolecules of the host microbiome before and after administration of a compound. The abundance of each biomolecule in the plurality may then be compared to determine if there is a decrease in the abundance of biomolecules associated with obesity after treatment.

In some embodiments, the array may be used as a diagnostic or prognostic tool to identify subjects that are susceptible to more efficient energy harvesting, and therefore, more susceptible to weight gain and/or obesity. Such a method may generally comprise incubating the array with biomolecules derived from the subject's gut microbiome to determine the relative abundance of Bacteroidetes or Firmictues. In some embodiments, the array may be used to determine the relative abundance of Mollicutes in a subject's gut microbiome. Methods to collect, isolate, and/or purify biomolecules from the gut microbiome of a subject to be used in the above methods are known in the art, and are detailed in the examples.

(b) microbiome Profiles

The present invention also encompasses use of the microbiome as a biomarker to construct microbiome profiles. Generally speaking, a microbiome profile is comprised of a plurality of values with each value representing the abundance of a microbiome biomolecule. The abundance of a microbiome biomolecule may be determined, for instance, by sequencing the nucleic acids of the microbiome as detailed in the examples. This sequencing data may then be analyzed by known software, as detailed in the examples, to determine the abundance of a microbiome biomolecule in the analyzed sample. The abundance of a microbiome biomolecule may also be determined using an array described above. For instance, by detecting the association between a biomolecules comprising a microbiome sample and the biomolecules comprising the array, the abundance of a microbiome biomolecule in the sample may be determined.

A profile may be digitally-encoded on a computer-readable medium. The term “computer-readable medium” as used herein refers to any medium that participates in providing instructions to a processor for execution. Such a medium may take many forms, including but not limited to non-volatile media, volatile media, and transmission media. Non-volatile media may include, for example, optical or magnetic disks. Volatile media may include dynamic memory. Transmission media may include coaxial cables, copper wire and fiber optics. Transmission media may also take the form of acoustic, optical, or electromagnetic waves, such as those generated during radio frequency (RF) and infrared (IR) data communications. Common forms of computer-readable media include, for example, a floppy disk, a flexible disk, hard disk, magnetic tape, or other magnetic medium, a CD-ROM, CDRW, DVD, or other optical medium, punch cards, paper tape, optical mark sheets, or other physical medium with patterns of holes or other optically recognizable indicia, a RAM, a PROM, and EPROM, a FLASH-EPROM, or other memory chip or cartridge, a carrier wave, or other medium from which a computer can read.

A particular profile may be coupled with additional data about that profile on a computer readable medium. For instance, a profile may be coupled with data about what therapeutics, compounds, or drugs may be efficacious for that profile. Conversely, a profile may be coupled with data about what therapeutics, compounds, or drugs may not be efficacious for that profile. Alternatively, a profile may be coupled with known risks associated with that profile. Non-limiting examples of the type of risks that might be coupled with a profile include disease or disorder risks associated with a profile. The computer readable medium may also comprise a database of at least two distinct profiles.

Such a profile may be used, for instance, in a method of selecting a compound for treating obesity or an obesity-related disorder in a host. Generally speaking, such a method would comprise providing a microbiome profile from the host and providing a plurality of reference microbiome profiles, each associated with a compound, and selecting the reference profile most similar to the host microbiome profile, to thereby select a compound for treating obesity or an obesity-related disorder in the host. The host profile and each reference profile may comprise a plurality of values, each value representing the abundance of a microbiome biomolecule.

The microbiome profiles may be utilized in a variety of applications. For example, the microbiome profiles may be used in a method for predicting risk for obesity or an obesity-related disorder in a host. The method comprises, in part, providing a microbiome profile from a host, and providing a plurality of reference microbiome profiles, then selecting the reference profile most similar to the host microbiome profile, such that if the host's microbiome is most similar to a reference obese microbiome, the host is at risk for obesity or an obesity-related disorder. The microbiome profile from the host may be determined using an array of the invention. The reference profiles may be stored on a computer-readable medium such that software known in the art and detailed in the examples may be used to compare the microbiome profile and the reference profiles.

The host microbiome may be derived from a subject that is a rodent, a human, a livestock animal, a companion animal, or a zoological animal. In one embodiment, the host microbiome is derived from a rodent, i.e. a mouse, a rat, a guinea pig, etc. In another embodiment, the host microbiome is derived from a human. In a yet another embodiment the host microbiome is derived from a livestock animal. Non-limiting examples of livestock animals include pigs, cows, horses, goats, sheep, llamas and alpacas. In still another embodiment, the host microbiome is derived from a companion animal. Non-limiting examples of companion animals include pets, such as dogs, cats, rabbits, and birds. In still yet another embodiment, the host microbiome is derived from a zoological animal. As used herein, a “zoological animal” refers to an animal that may be found in a zoo. Such animals may include non-human primates, large cats, wolves, and bears.

### (c) Kits

The present invention also encompasses a kit for evaluating a compound, therapeutic, or drug. Typically, the kit comprises an array and a computer-readable medium. The array may comprise a substrate, the substrate having disposed thereon at least one biomolecule that is modulated in an obese host microbiome compared to a lean host microbiome. The computer-readable medium may have a plurality of digitally-encoded profiles wherein each profile of the plurality has a plurality of values, each value representing the abundance of a biomolecule in a host microbiome detected by the array. The array may be used to determine a profile for a particular host under particular conditions, and then the computer-readable medium may be used to determine if the profile is similar to known profile stored on the computer-readable medium. Non-limiting examples of possible known profiles include obese and lean profiles for several different hosts, for example, rodents, humans, livestock animals, companion animals, or zoological animals.

### DEFINITIONS

The term “abundance” refers to the representation of a given phylum, order, family, or genera of microbe present in the gastrointestinal tract of a subject.

The term “activity of the microbiota population” refers to the microbiome's ability to harvest energy.

The term “antagonist” refers to a molecule that inhibits or attenuates the biological activity of a Fiaf polypeptide and in particular, the ability of Fiaf to inhibit LPL. Antagonists may include proteins such as antibodies, nucleic acids, carbohydrates, small molecules, or other compounds or compositions that modulate the activity of a Fiaf polypeptide either by directly interacting with the polypeptide or by acting on components of the biological pathway in which Fiaf participates.

The term “agonist” refers to a molecule that enhances or increases the biological activity of a Fiaf polypeptide and in particular, the ability of Fiaf to inhibit LPL. Agonists may include proteins, peptides, nucleic acids, carbohydrates, small molecules (e.g., such as metabolites), or other compounds or compositions that modulate the activity of a Fiaf polypeptide either by directly interacting with the polypeptide or by acting on components of the biological pathway in which Fiaf participates.

The term “altering” as used in the phrase “altering the microbiota population” is to be construed in its broadest interpretation to mean a change in the representation of microbes in the gastrointestinal tract of a subject. The change may be a decrease or an increase in the presence of a particular microbial species, genus, family, order, or class.

“BMI” as used herein is defined as a human subject's weight (in kilograms) divided by height (in meters) squared.

An “effective amount” is a therapeutically-effective amount that is intended to qualify the amount of agent that will achieve the goal of a decrease in body fat, or in promoting weight loss.

Fas stands for fatty acid synthase.

Fiaf stands for fasting-induced adipocyte factor.

LPL stands for lipoprotein lipase.

The term “obesity-related disorder” includes disorders resulting from, at least in part, obesity. Representative disorders include metabolic syndrome, type II diabetes, hypertension, cardiovascular disease, and nonalcoholic fatty liver disease.

The term “metagenomics” refers to the application of modern genomics techniques to the study of communities of microbial organisms directly in their natural environments, by passing the need for isolation and lab cultivation of individual species.

PPAR stands for peroxisome proliferator-activator receptor.

A “subject in need of treatment for obesity” generally will have at least one of three criteria: (i) BMI over 30; (ii) 100 pounds overweight; or (iii) 100% above an “ideal” body weight as determined by generally recognized weight charts.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate various iterations of the invention.

### Example 1

### Shotgun Sequencing of Microbiomes

To determine if microbial community gene content correlates with, and is a potential contributing factor to obesity, we characterized the distal gut microbiome of adult C57BL/6J mice homozygous for a mutation in the leptin gene (ob) that produces obesity, as well as the microbiomes of their lean (ob/+ and +/+) littermates by random shotgun sequencing of their cecal microbial DNA. Mice were used for these comparative metagenomics studies to eliminate many of the confounding variables (environment, diet, and genotype) that would make such a proof-of-principle experiment more difficult to perform and interpret in humans. The cecum was chosen as the gut habitat for sampling because it is an anatomically distinct structure, located between the distal small intestine and colon that is colonized with sufficient quantities of a readily harvested microbiota for metagenomic analysis.

Animals. All experiments involving mice were performed using protocols approved by the Washington University Animal Studies Committee. Once C57BL/6J ob/ob, ob/+, and +/+ littermates were weaned, they were housed individually in microisolator cages where they were maintained in a specified pathogen-free state, under a 12-h light cycle, and fed a standard polysaccharide-rich chow diet (PicoLab, Purina) ad libitum. Germ-free and colonized animals were maintained in gnotobiotic isolators, under a strict 12-h light cycle and fed an autoclaved chow diet (B&K Universal, East Yorkshire, U.K.) ad libitum. Fecal samples for bomb calorimetry were collected from mice at 8 or 14 weeks of age, after which time animals were sacrificed.

Community DNA Preparation The cecal contents used for community DNA sequencing and gas chromatography-mass spectrometry (GC-MS) were obtained, at eight weeks of age, from the same animals used for a previous PCR-based 16S rRNA survey of the gut microbiota (Ley et al. (2005) Proc. Natl. Acad, Sci. USA 102:11070-11075): samples had been stored at −80° C. (Table 1). An aliquot (−10 mg) of each sample was suspended while frozen in a solution containing 500 μL of extraction buffer [200 mM Tris (pH 8.0), 200 mM NaCl, 20 mM EDTA], 210 μL of 20% SDS, 500 μL of a mixture of phenol:chloroform:isoamyl alcohol (25:24:1)], and 500 μL of a slurry of 0.1-mm-diameter zirconia/silica beads (BioSpec Products, Bartlesville, Okla.). Microbial cells were then lysed by mechanical disruption with a bead beater (BioSpec Products) set on high for 2 min (23° C.), followed by extraction with phenol:chloroform:isoamyl alcohol, and precipitation with isopropanol. In order to perform pyrosequencing, DNA was purified further using the Qiaquick gel extraction kit (Qiagen).

Shotgun sequencing and assembly of cecal microbiomes DNA samples were used to construct plasmid libraries for 3730x1 capillary-based sequencing. Pyrosequencing was performed as previously described (Margulies at al. (2005) Nature 437:376-380). Briefly, samples were nebulized to 200 nucleotide fragments, ligated to adaptors, fixed to beads, suspended in a PCR reaction mixture-in-oil emulsion, amplified, and sequenced using a GS20 pyrosequencer (454 Life Sciences, Branford, C T). The Newbler de novo shotgun sequence assembler (454 Life Sciences) was used to assemble sequences based on flowgram signal space. This process included overlap generation, contig layout, and consensus generation. The resulting GS20 contigs were then broken into linked sequences to generate pseudo paired-end reads, and aligned with 3730x1 reads using PCAP (Huang et al. (2003) Genome Res. 13:2164-2170).

Sequences were aligned to reference genomes using the PROmer script in MUMmer Kurtz et al. (2004) Genome Biol. 5:R12) (version 3.18). Capillary sequencer reads from each microbiome, the finished genome of the human gut-derived Bacteroides thetaiotaomicron type strain ATCC29148 (Xu et al. (2003) Science 299:2074-2076), and a deep draft genome of the human gut-derived Eubacterium rectale type strain ATCC33656 (http://gordonlab.wustl.edu/supplemental/Turnbaugh/obob/) were used as a reference for the pyrosequencer datasets. Coverage was calculated by dividing the sum of all alignment lengths by the length of the reference genome.

Whole genome sequencing and annotation A draft assembly of Eubacterium rectale ATCC33656 was generated from AB37301x1 paired end-reads of inserts in whole genome shotgun plasmid and fosmid libraries, as well as from reads produced by the GS20 pyrosequencer. Sequences were assembled using Newbler and PCAP (see above) and ORFS predicted with Glimmer3.01 (Delcher et al. (1999) Nucl. Acids Res, 27:4636-4641) (maximum overlap of 100, minimum length of 110 and a threshold of 30). Each predicted gene sequence was translated, and the resulting protein sequence assigned to InterPro numbers using InterProScan (Mulder et al. (2005) Nucl. Acids Res. 33:D201-205) (Release 12.0).

TABLE 1
Nomenclature used to designate metagenomic datasets
obtained from the cecal microbiota of C57BL/6J
ob/ob, ob/+, and +/+ littermates.

| | | | 16S rRNA | | |
|---|---|---|---|---|---|
| | Metagenome | | survey | | Host |
| Figure label | label | Litter | label¹ | Tree label¹ | genotype |
| ob1 | PT6 | 1 | C23 | M2B-4 | ob/ob |
| ob2 | PT4 | 2 | C18 | M1-2 | ob/ob |
| lean1 | PT3 | 1 | C21 | M2B-1 | +/+ |
| lean2 | PT8 | 2 | C15 | M1-3 | ob/+ |
| lean3 | PT2 | 2 | C16 | M1-4 | +/+ |

¹Samples from previous 16S rRNA survey: (Ley et al. (2005) Proc. Natl. Acad. Sci. USA 102: 11070-11075.

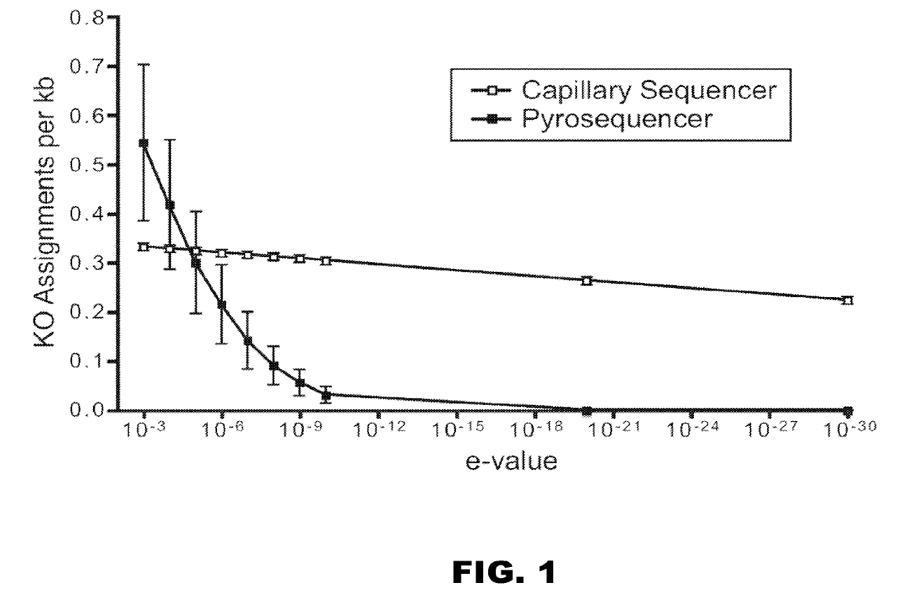

Results. Bulk DNA was prepared from the cecal contents of two ob/ob and +/+ littermate pairs. A lean ob/+ mouse from one of the litters was also studied. All cecal microbial community DNA samples were analyzed using a 3730x1 capillary sequencer [10,500±31 unidirectional reads/dataset; 752±13.8 (s.e.m.) nucleotides/read; 39.5 Mb from all five plasmid libraries]. Material from one of the two obese and lean sibling pairs was also analyzed using a highly parallel 454 Life Sciences GS20 pyrosequencer: three runs for the +/+ mouse (known as lean1), and two runs for its ob/ob littermate (ob1) produced a total of 160 Mb of sequence [345,000±23,500 unidirectional reads/run; 93.1±1.56 nucleotides/read] (Tables 2 and 3). Both sequencing platforms have unique advantages and limitations: capillary sequencing allows more confidant gene calling (FIG. 1) but is affected by cloning bias while pyrosequencing can achieve higher sequence coverage with no cloning bias, but produces shorter reads (Table 2). The three pyrosequencer runs of the lean1 cecal microbiome (94.9 Mb) yielded 0.44× coverage (based on PROmer sequence alignments) of the 3730x1-derived sequences obtained from the same sample (8.23 Mb), while the two pyrosequencer runs of the microbiome of its ob/ob littermate (ob1, 65.4 Mb) produced 0.32× coverage of the corresponding 3730x1 sequences (8.19 Mb).

TABLE 2
Sequencing results for each cecal microbiome.

| | | Average | | |
|---|---|---|---|---|
| | | read | Number | |
| Microbiome | Sequencer | length | of reads | Sequence |
| lean1 | GS20 | 90.9 | 1,046,611 | 94,913,476 |
| ob1 | GS20 | 96.4 | 677,384 | 65,370,448 |
| lean1 | 3730x1 | 765 | 10,752 | 8,227,047 |
| lean2 | 3730x1 | 782 | 11,136 | 8,705,876 |
| lean3 | 3730x1 | 706 | 10,752 | 7,590,528 |
| ob1 | 3730x1 | 735 | 11,136 | 8,185,880 |
| ob2 | 3730x1 | 771 | 8,832 | 6,811,035 |
| TOTAL | | | 1,776,603 | 199,804,290 |

TABLE 3
Assembly of reads from capillary sequencer
and pyrosequencer datasets.

| | | | | | | N50 |
|---|---|---|---|---|---|---|
| | | | Average | | Largest | contig |
| Sam- | | | contig | Contiged | Assem- | length |
| ple | Sequencer | Contigs | length | bases¹ | bly | (kb)² |
| lean1 | GS20 | 102,299 | 117 | 11,966,580 | 2,793 | 0.109 |
| ob1 | GS20 | 56,425 | 116 | 6,518,469 | 2,174 | 0.109 |
| lean1 | 3730x1 | 167 | 1527 | 254,985 | 5,500 | 1.62 |
| lean2 | 3730x1 | 407 | 1598 | 650,499 | 5,522 | 1.71 |
| lean3 | 3730x1 | 224 | 1528 | 342,172 | 3,281 | 1.59 |
| ob1 | 3730x1 | 320 | 1393 | 445,814 | 3,225 | 1.49 |
| ob2 | 3730x1 | 269 | 1644 | 442,210 | 4,186 | 1.70 |
| All | 3730x1 | 2,575 | 1734 | 4,465,685 | 11,213 | 1.78 |
| All | GS20 | 159,245 | 118 | 18,809,438 | 2,708 | 0.110 |
| All | GS20 and | 13,667 | 898 | 12,275,469 | 14,755 | 0.903 |
| | 3730x1 | | | | | |

¹Contiged bases refers to the combined length of all contigs.
²N50 contig length refers to the length of the contig, such that 50% of the total contiged bases are present in contigs of greater or equal size.

### Example 2

### Taxonomic Analysis of Microbiomes

Database search parameters NCBI BLAST was used to query the nonredundant database (NR), the STRING-extended COG database (179 microbial genomes, version 6.3) (von Mering et al. (2005) Nucl. Acids Res. 33:D433-437), a database constructed from 334 genomes available through KEGG (version 37) (Kanehisa et al. (2004) Nucl. Acids Res 32: D277-280), and the Ribosomal Database Project database (RDP, version 9.33) (Cole et al. (2005) Nucl. Acids Res33:D294-296). Reads with multiple COG/KO hits were counted once for each classification scheme. KO hits were also categorized into CAZy families (http://afmb.cnrs-mrs.fr/CAZY/). KEGG pathway maps are available on-line (http://gordonlab.wustl.edu/supplemental/Turnbaugh/obob/). NR, COG, and KEGG comparisons were performed using NCBI BLASTX. RDP comparisons were performed using NCBI BLASTN, and microbiomes were directly compared using TBLASTX. A cutoff of e-value <10⁻⁵ was used for EGT assignments and sequence comparisons DeLong et al. (2006) Science 311:496-503) (corresponds to a p-value cutoff of 10⁻¹² against the NR and KEGG databases, and 10⁻¹¹ against the COG database). Given this cutoff, we would only expect three false EGT assignments in our combined analyses due to random chance. We also re-analyzed the data using a more stringent cutoff (Tringe et al. (2005) Science 308:554-557) (e-value <10⁻⁸).

Taxonomic assignments of shotgun 16S rRNA gene fragments Shotgun reads containing a 16S rRNA fragment were identified by BLASTX comparison of each microbiome to the RDP database. 16S rRNA gene fragments were then aligned using the NASTA multi-aligner (DeSantis et al. (2006) Nucl. Acids Res. 34:W394-399) with a minimum template length of 20 bases and a minimum percent identity of 75%. The resulting alignment was then imported into an ARB neighbor-joining tree and hypervariable regions were masked using the lanemaskPH filter (Ludwig et al. (2004) Nucl. Acids Res. 32:1363-1371). Direct BLAST taxonomic assignments were performed through BLASTX comparisons of each microbiome and the NR database. Best-BLAST-hits with an e-value <10⁻⁵ were used to assign each read to a given species.

Estimating the total number of orthologous groups The total estimated number of COGs and NOGs (Non-supervised Orthologous Groups) in each sample was calculated using the lower-limit of the Chao1 95% confidence interval in EstimateS (Version 7.5, R. K. Colwell, http://purl.ocic.org/estimates), based on the number of EGTs assigned to each orthologous group. The number of missed groups was calculated by subtracting the estimated total (Chao1 lower-limit) from the observed number of groups.

Direct comparisons of microbiome sequences Microbiomes sequenced using the 3730x1 instrument were evaluated by reciprocal pairwise TBLASTX comparisons (DeLong et al. (2006) Science 311:496-503). 8,832 reads were used from each microbiome to limit artifacts that arise from different sized datasets. Each possible pairwise comparison was made by using a BLAST database constructed from each microbiome. Samples were clustered based on the cumulative pairwise BLAST score. An estimate of distance was constructed using the D2 normalization and genome conservation approach previously used for genome clustering (Kunin et al. (2005) Nucl. Acids Res. 33:616-621) This method calculates a distance score based on the minimum cumulative BLAST score (sum of all best-BLAST-hit scores) between two microbiomes and the weighted average of both self-self comparisons (D2=−ln(min S₁ᵥ₂, S₂ᵥ₁/average). The weighted average is calculated using average=squareroot(2)*S₁ᵥ₁*S₂ᵥ₂/squareroot (S₁ᵥ₁²+S₂ᵥ₂²). The resulting distances were used to create a distance matrix. A tree was constructed using NEIGHBOR (PHYLIP version 3.64; kindly provided by J. Felsenstein, Department of Genome Sciences, University of Washington, Seattle), and was viewed using Treeview X (Page (1996) Comput. Appl. Biosci. 12:357-358).

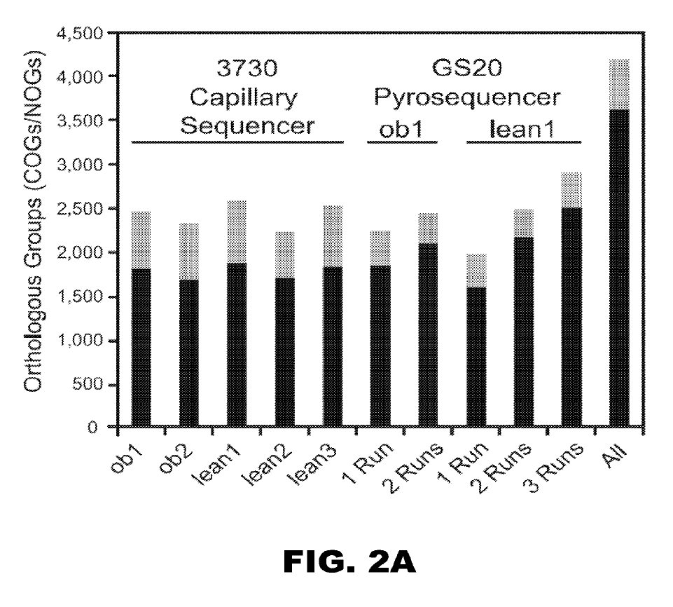

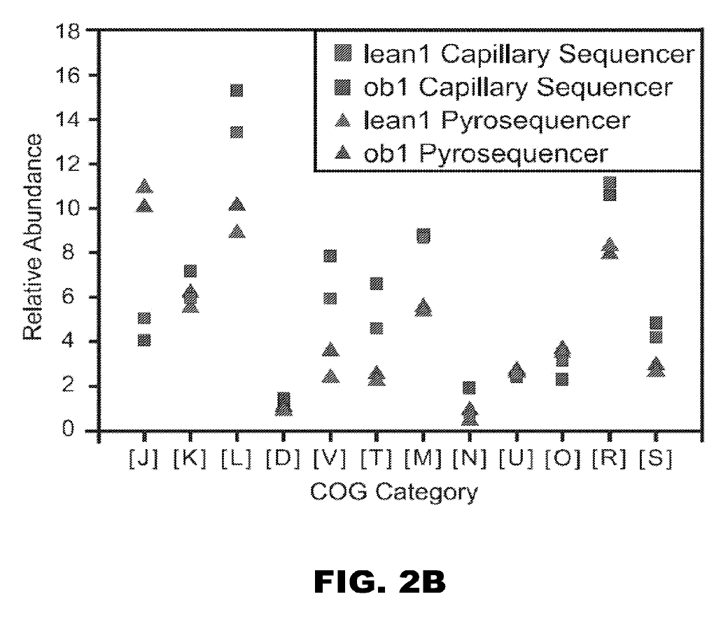

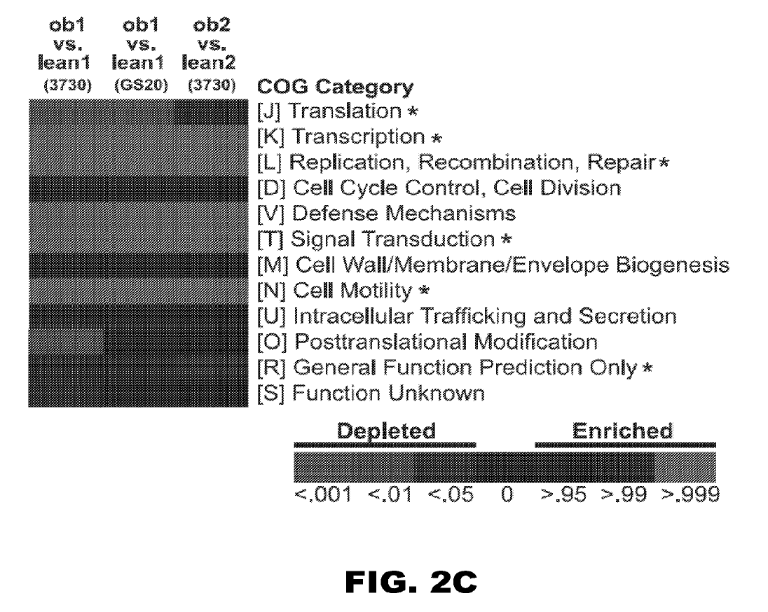

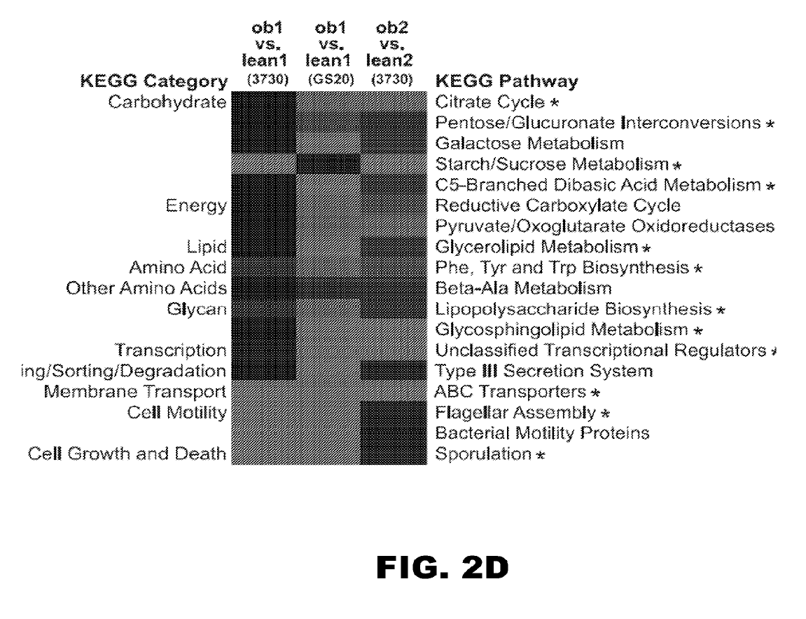

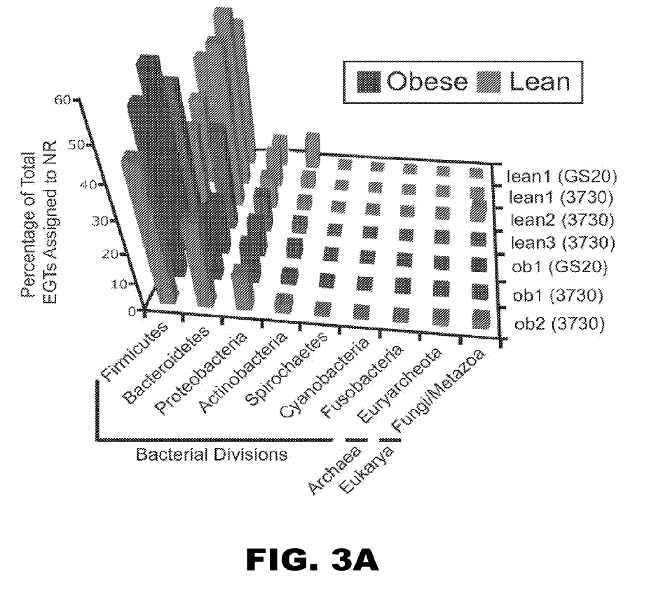

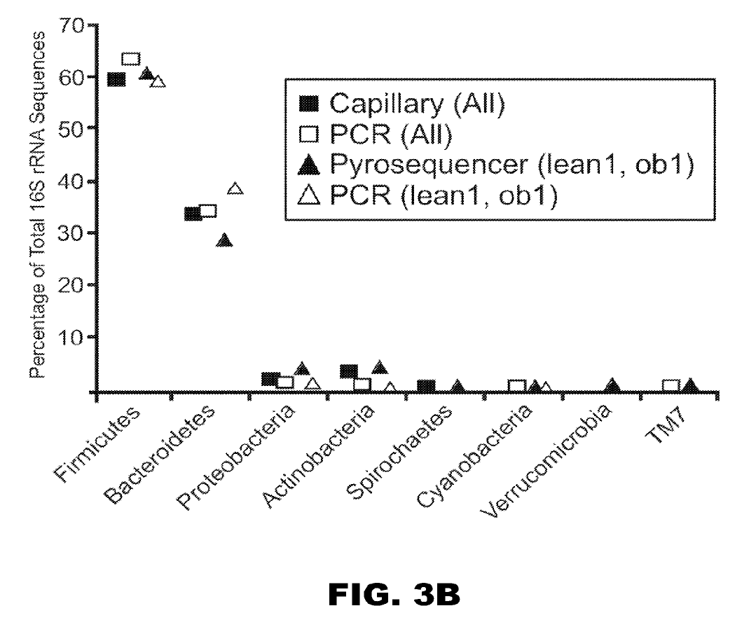

Results. Environmental gene tags (EGTs) are defined as sequencer reads assigned to the NCBI non-redundant (NR), Clusters of Orthologous Groups (COG), or Kyoto Encyclopedia of Genes and Genomes (KEGG) databases (FIG. 2A; FIG. 3 Table 4). Averaging results from all datasets, 94% of the EGTs assigned to NR were bacterial, 3.6% were eukaryotic (0.29% Mus musculus; 0.36% fungal), 1.5% were archaeal (1.4% Euryarcheota; 0.07% Crenarcheota), and 0.61% were viral (0.57% dsDNA viruses) (Table 5). The relative abundance of the eight bacterial divisions identified from EGTs and 16S rRNA gene fragments was comparable to our previous PCR-derived, 16S rRNA gene sequence-based surveys of these cecal samples, including the increased ratio of Firmicutes to Bacteroidetes in obese versus lean littermates (FIG. 3). In addition, comparisons of the lean1 and ob1 reads obtained with the pyrosequencer against the finished genome of B. thetaiotaomicron ATCC29148, and a deep draft genome assembly of Eubacterium rectale ATCC33656 (N50 contig size 75.9 kB; http://gordonlab.wustl.edu/supplemental/Turnbaugh/obob/) provided independent confirmation of the greater relative abundance of Firmicutes in the ob/ob microbiota. These organisms were selected for comparison because both are prominently represented in the normal human distal gut microbiota (Eckburg et al. (2005) Science 308:1635-1638) while species related to B. thetaiotaomicron and E. rectale are members of the normal mouse distal gut microbiota (Gill et al. (2006) Science 312:1355-1359). The ratio of sequences homologous to the E. rectale versus B. thetaiotaomicron genome was 7.3 in the ob1 cecal microbiome compared to 1.5 in the lean1 microbiome.

There were more EGTs that matched Archaea (Euryarchaeota and Crenarchaeota) in the cecal microbiome of ob/ob mice compared to their lean ob/+ and +/+ littermates (binomial test of pooled obese versus pooled lean capillary sequencing derived microbiomes, P<0.001) (Table 5). Methanogenic archaea increase the efficiency of bacterial fermentation by removing one of its end products, H₂. Our recent studies of gnotobiotic normal mice colonized with the principal methanogenic archaeon in the human gut, Methanobrevibacter smithii, and/or B. thetaiotaomicron revealed that co-colonization not only increases the efficiency, but also changes the specificity of bacterial polysaccharide fermentation, leading to a significant increase inadiposity compared with mice colonized with either organism alone (Samuel and Gordon (2006) Proc. Natl. Acad. Sci. USA 103:10011-10016).

TABLE 4
Number of EGTs assigned to the
NR, COG, and/or KEGG databases.

| | | | | | Percent |
|---|---|---|---|---|---|
| | Total NR | Total COG | Total KO | Total | unas- |
| Microbiome | EGTs | EGTs | EGTs | EGTs | signed |
| lean1 (GS20) | 48,625 | 51,481 | 28,359 | 56,599 | 94.6 |
| ob1 (GS20) | 33,360 | 32,819 | 18,308 | 39,058 | 94.2 |
| lean1 (3730xl) | 7,973 | 7,970 | 2,810 | 8,462 | 21.3 |
| lean2 (3730xl) | 7,309 | 7,687 | 2,723 | 8,170 | 26.6 |
| lean3 (3730xl) | 7,042 | 7,119 | 2,562 | 7,616 | 29.2 |
| ob1 (3730xl) | 7,331 | 7,299 | 2,639 | 7,859 | 29.4 |
| ob2 (3730xl) | 6,008 | 6,016 | 2,053 | 6,425 | 27.3 |

TABLE 5
Percentage of total assigned reads among each taxonomic domain based on BLASTX
searches of the NR database with an e-value cutoff ≦10⁻⁵.

| | lean1 | lean1 | lean2 | lean3 | ob1 | ob1 | ob2 |
|---|---|---|---|---|---|---|---|
| Domain | 3730x1 | GS20 | 3730x1 | 3730x1 | 3730x1 | GS20 | 3730x1 |
| Archaea | 1.28 | 0.658 | 1.55 | 1.59 | 2.07 | 1.23 | 2.08 |
| Bacteria | 95.8 | 97.9 | 90.7 | 95.1 | 94.4 | 93.4 | 92.9 |
| Eukaryota | 2.36 | 1.39 | 7.36 | 2.74 | 2.77 | 4.15 | 4.19 |
| (Viruses) | 0.527 | 0.065 | 0.383 | 0.611 | 0.709 | 1.21 | 0.782 |

### Example 3

### Comparative Metagenomic Analysis

Clustering of microbiomes based on predicted metabolic function Microbiomes were clustered based on the percent representation of EGTs assigned to each COG, KEGG pathway, and phylotype (genome in NR) using Cluster3.0. Percent representation was calculated as the number of EGTs assigned to a given group divided by the number of EGTs assigned to all groups. Single linkage hiearchical clustering via Pearson's correlation was performed on each dataset, and the results were visualized by using the Treeview Java applet (Saldanha (2004) Bioinformatics 20:3246-3248). Principal Component Analysis was also performed based on the percent representation of EGTs assigned to KEGG pathways (Cluster3.0) (Dailey et al. (1987) J. Bacteriol. 169:917-919), and the data were graphed according to the first two coordinates.

Identification of statistically enriched and depleted metabolic groups Two methods were used to determine statistically enriched or depleted metabolic groups: the cumulative binomial distribution ((Gill et al. (2006) Science 312:1355-1359) and a bootstrap analysis (DeLong et al. (2006) Science 311:496-503; Rodriguez-Brito et al. (2006) BMC Bioinformatics 7:162). The cumulative binomial distribution was used for pairwise comparisons of microbiome COG, KEGG, and taxonomic assignments. The calculation uses the following inputs: number of successes for microbiome 1 (number of EGTs assigned to a given group), number of trials for microbiome 1 (total number of EGTs assigned to all groups), and the expected frequency (number of successes/number of trials for microbiome 2). The probability of having less than or equal to the number of observed EGTs in a given group was then calculated using the cumulative binomial distribution. Depletion was defined as having a probability less than 0.05, 0.01, or 0.001 assuming p equals the expected frequency and that the expected frequency is normally distributed. Enrichment was defined as having a probability of greater than 0.95, 0.99, or 0.999 given the same assumptions. To minimize false negatives, no corrections for multiple sampling were made. To limit false positives resulting from low sampling, only groups with at least one hit in each microbiome were evaluated.

Xipe (Rodriguez-Brito, version 0.2) (Rodriguez-Brito et al. (2006) BMC Bioinformatics 7:162) was employed for bootstrap analyses of KEGG pathway enrichment and depletion, using the following parameters: 10,000 samples, 10,000 repeats, and three confidence levels (95%, 99%, and 99.9%). Briefly, a dataset composed of the number of EGTs assigned to each KEGG pathway was sampled with replacement from each microbiome 10,000 times. The difference between the number of EGTs per pathway in the first microbiome, and the number of EGTs per pathway in the second microbiome, was calculated for each group. This process was repeated 10,000 times and the median difference calculated for each pathway. A confidence interval was determined by pooling both datasets and comparing 10,000 random samples to 10,000 other random samples. Groups with a larger median difference between microbiomes than the confidence interval were considered significantly different.

Biochemical analyses—Short-chain fatty acids (SCFAs) were measured in nine cecal samples (4 lean, 5 obese) obtained from nine mice that had been used for our previous 16S rRNA gene sequence-based survey [animals C1, C3, C4, C9, C10, C13, C15 (lean2), C17, and C22 (Ley et al. (2005) Proc. Natl. Acad. Sci. USA 102:11070-11075)]. Two aliquots of each sample were evaluated. SOFA levels were quantified according to previously published protocols (Samuel and Gordon (2006) Proc. Natl. Acad. Sci. USA 103:10011-10016): i.e., double diethyl ether extraction of deproteinized cecal contents spiked with isotope-labeled internal SOFA standards; derivatization of SCFAs with N-tert-butyldimethylsilyl-Nmethyltrifluoracetamide (MTBSTFA); and GC-MS analysis of the resulting TBDMS derivatives.

Bomb calorimetry was performed on 44 fecal samples collected from 22 mice (9 lean, 13 obese). Each mouse was transferred to a clean cage for 24 hours, at which point fecal samples were collected and oven dried at 60° C. for 48 hours. Gross energy content was measured using a semimicro oxygen bomb calorimeter, calorimetric thermometer, and semimicro oxygen bomb (Models 6725, 6772 and 1109, respectively, from Parr Instrument Co.). The calorimeter energy equivalent factor was determined using benzoic acid standards. The mean of each distribution was compared using a two-tailed Student's t-Test (p<0.05).

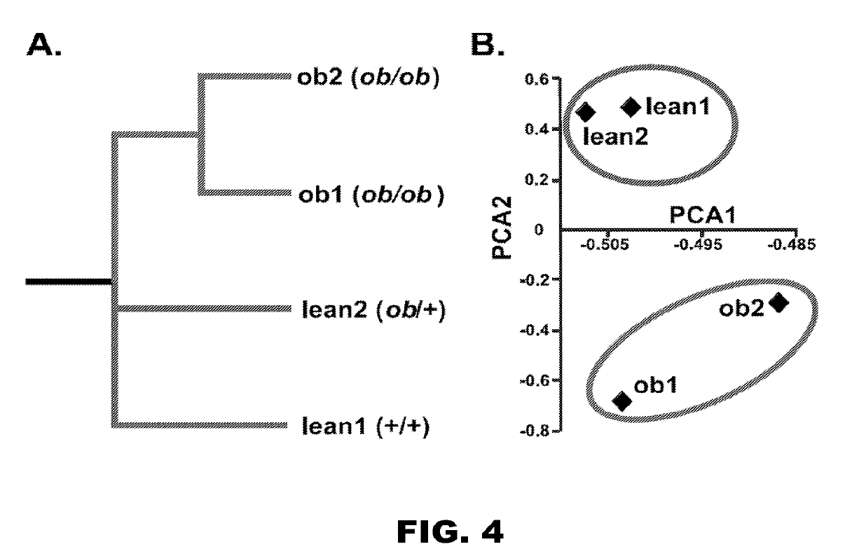

Results. Using reciprocal TBLASTX comparisons, we found that the Firmicutes-enriched microbiomes from ob/ob hosts clustered together (FIG. 4A). Likewise, Principal Component Analysis of EGT assignments to KEGG pathways revealed a correlation between host genotype and the gene content of the microbiome (FIG. 4B). Reads were then assigned to COGs and KOs (KEGG orthology terms) by BLASTX comparisons against the STRING-extended COG database, and the KEGG Genes database (version 37). We tallied the number of EGTs assigned to each COG or KEGG category, and used the cumulative binomialdistribution, and a bootstrap analysis, to identify functional categories with significant differences in their representation in both sets of obese and lean littermates.

As noted above, capillary sequencing requires cloned DNA fragments: the pyrosequencer does not, but produces relatively short read lengths. These differences are a likely cause of the shift in relative abundance of several COG categories obtained using the two sequencing methods for the same sample (FIG. 2B). Nonetheless, comparisons of the cecal microbiomes of lean versus obese littermates sequenced with either method revealed similar differences in their functional profiles (FIG. 2C).

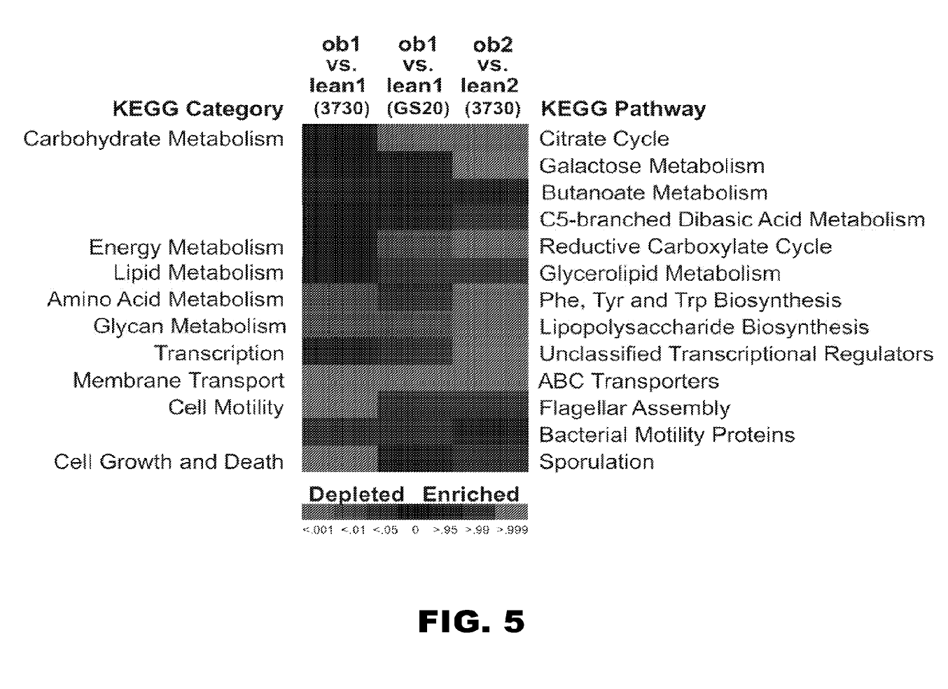

The ob/ob microbiome is enriched for EGTs encoding many enzymes involved in the initial steps in breaking down otherwise indigestible dietary polysaccharides, including KEGG pathways for starch/sucrose metabolism, galactose metabolism, and butanoate metabolism (FIGS. 2D, 5; Table 6). EGTs representing these enzymes were grouped according to their functional classifications in the Carbohydrate Active Enzymes (CAZy) database (http://afmb.cnrsmrs.fr/CAZY/). The ob/ob microbiome is enriched (P<0.05) for eight glycoside hydrolase families capable of degrading dietary polysaccharides including starch (Families 2, 4, 27, 31, 35, 36, 42 and 68 which contain alpha-glucosidases, alphagalactosidases, and beta-galactosidases). Finished genome sequences of prominent human gut Firmicutes have not been reported. However, our analysis of the draft genome of E. rectale has revealed 44 glycoside hydrolases, including a significant enrichment for glycoside hydrolases involved in the degradation of dietary starches [CAZy Families 13 and 77 which contain alpha-amylase and amylomaltase; P<0.05 based on binomial test of E. rectale versus the finished genomes of Bacteroidetes (Bacteroides thetaiotaomicron ATCC29148, B. fragilis NCTC9343, B. vulgatus ATCC8482 and B. distasonis ATCC8503].

EGTs encoding proteins that import the products of these glycoside hydrolases (ABC transporters), metabolize them [e.g., alpha- and beta-galactosidases (KO7406/7 and KO1190)], and generate the major end products of fermentation, butyrate and acetate [KEGG ‘Butanoate metabolism’ pathway; pyruvate formate-lyase (KO0656); and formate-tetrahydrofolate ligase (KO1938; second enzyme in the homoacetogenesis pathway for converting CO2 to acetate)], are also significantly enriched in the ob/ob microbiome (binomial comparison of pyrosequencer-derived ob1 and lean1 datasets, P<0.05) (FIGS. 2D, 5; Table 6).

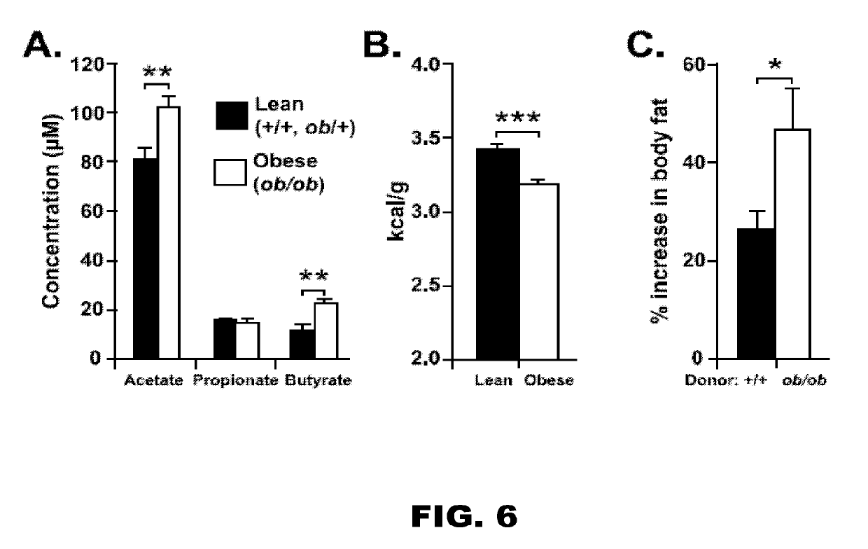

As predicted from our comparative metagenomic analyses, the ob/ob cecum has an increased concentration of the major fermentation end-products butyrate and acetate (FIG. 6A). This observation is also consistent with the fact that many Firmicutes are butyrate producers. Moreover, bomb calorimetry revealed that ob/ob mice have significantly less energy remaining in their feces relative to their lean littermates (FIG. 6B).

TABLE 6
KEGG pathways enriched in the pooled ob/ob cecal microbiome
relative to the pooled lean cecal microbiome (capillary sequencing
datasets, ob1 + ob2 vs. lean1 + lean2 + lean3, binomial
test, P < 0.05).

| KEGG Category | KEGG Pathway¹ |
|---|---|
| Carbohydrate Metabolism | Starch and sucrose metabolism |
| | Aminosugars metabolism |
| | Nucleotide sugars metabolism |
| Amino Acid Metabolism | Lysine biosynthesis |
| Metabolism of Other Amino Acids | D-Alanine metabolism |
| Glycan Biosynthesis and | N-Glycan degradation |
| Metabolism | |
| | Glycosaminoglycan degradation |
| | Glycosphingolipid metabolism |
| Biosynthesis of Polyketides and | Polyketide sugar unit biosynthesis |
| Nonribosomal Peptides | |
| | Biosynthesis of vancomycin group |
| | antibiotics |
| Transcription | Other and unclassified family |
| | transcriptional |
| | regulators |
| Folding, Sorting and Degradation | Type III secretion system |
| Membrane Transport | ABC transporters |
| | Phosphotransferase system (PTS) |
| Signal Transduction | Two-component system |
| Cell Motility | Bacterial chemotaxis |
| | Flagellar assembly |
| | Bacterial motility proteins |
| Cell Growth and Death | Sporulation |

¹Only pathways with greater than ten hits in both pooled datasets are shown.

### Example 4

### Microbiota Transplantation

Microbiota transplantation experiments Germ-free C57BL/6J mice (8-9 weeks old) were colonized with a cecal microbiota obtained from either a lean (+/+) or an obese (ob/ob) C57BL/6J donor (n=1 donor and 4-5 recipients/treatment group/experiment; 2 independent experiments). Recipient mice were anesthetized at 0 and 14 days post colonization with an i.p. injection of ketamine (10 mg/kg body weight) and xylazine (10 mg/kg) and total body fat content was measured by dual-energy x-ray absorptiometry (Lunar PIXImus Mouse, GE Medical Systems) using previously described protocols (Bernal-Mizrachi et al (2002) Arterioscler. Thromb. Vasc. Biol. 22:961-968). Donor mice were sacrificed at day 0 and recipient mice after the final DEXA on day 14.

16S rRNA sequence-based surveys of the cecal microbiotas of conventionalized mice Cecal contents were recovered at the time of sacrifice by manual extrusion and frozen immediately at −80° C. DNA was prepared by bead beating, phenol/chloroform extraction, and gel purification (see above). Five replicate PCRs were performed for each mouse. Each 25 μl reaction contained 50-100 ng of purified DNA from cecal contents, 10 mM Tris (pH 8.3), 50 mMKCl, 2 mM MgSO4, 0.16 μM dNTPs, 0.4 μM of the bacteria-specific primer 8F (5′-AGAGTTTGATCCTGGCTCAG-3′), 0.4 μM of the universal primer 1391R (5′-GACGGGCGGTGWGTRCA-3′), 0.4 M betaine, and 3 units of Taq polymerase (Invitrogen). Cycling conditions were 94° C. for 2 min, followed by 35 cycles of 94° C. for 1 min, 55° C. for 45 sec, and 72° C. for 2 min, with a final extension period of 20 min at 72° C. Replicate PCRs were pooled, concentrated with Millipore columns (Montage), gel-purified with the Qiaquick kit (Qiagen), cloned into TOPO TA pCR4.0 (Invitrogen), and transformed into E. coli TOP10 (Invitrogen). For each mouse, 384 colonies containing cloned amplicons were processed for sequencing.

Plasmid inserts were sequenced bidirectionally using vector-specific primers and the internal primer 907R (5′-CCGTCAATTCCTTTRAGTTT-3′). 16S rRNA gene sequences were edited and assembled into consensus sequences using the PHRED and PHRAP software packages within the Xplorseq program (Papineau et al. (2006) Appl. Environ. Microbiol. 71:4822-4832). Sequences that did not assemble were discarded and bases with PHRED quality scores <20 were trimmed. Sequences were checked for chimeras using Bellerophon (Huber et al. (2004) Bioinformatics 20:2317-2319) and sequences with greater than 95% identity to both parents were removed (n=535; 13% of aligned sequences). The final dataset (n=4,157 sequences; for ARB alignment and tree see http://gordonlab.wustl.edu/supplemental/Turnbaugh/obob/; for sequence designations see Table 7) was aligned using the on-line version of the NAST multialigner (DeSantis et al. (2006) Nucl. Acid Res. 34:W394-399) (minimum alignment length=1250; percent identity >75), hypervariable regions were masked using the lanemaskPH filter provided with the ARB database (Ludwig et al. (2004) Nucl. acid Res. 32: 1363-1372, and the aligned sequences were added to the ARB neighbor-joining tree (based on pairwise distances with the Olsen correction) with the parsimony insertion tool. A phylogenetic tree containing all 16S rRNA gene sequences was exported from ARB and clustered using online UniFrac (Lozupone et al. (2006) BMC Bioiniformatics 7:371) without abundance weighting.

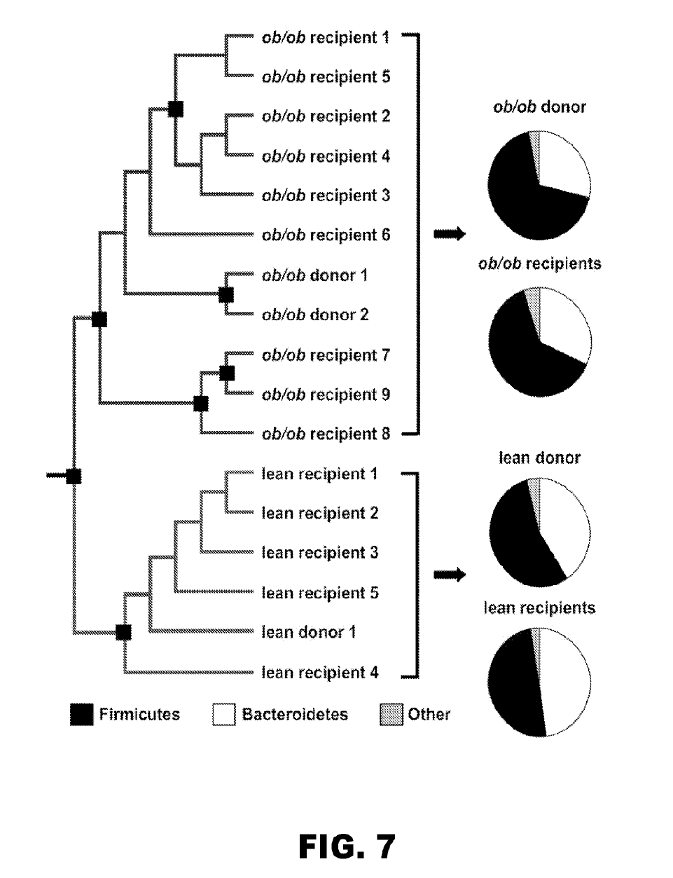

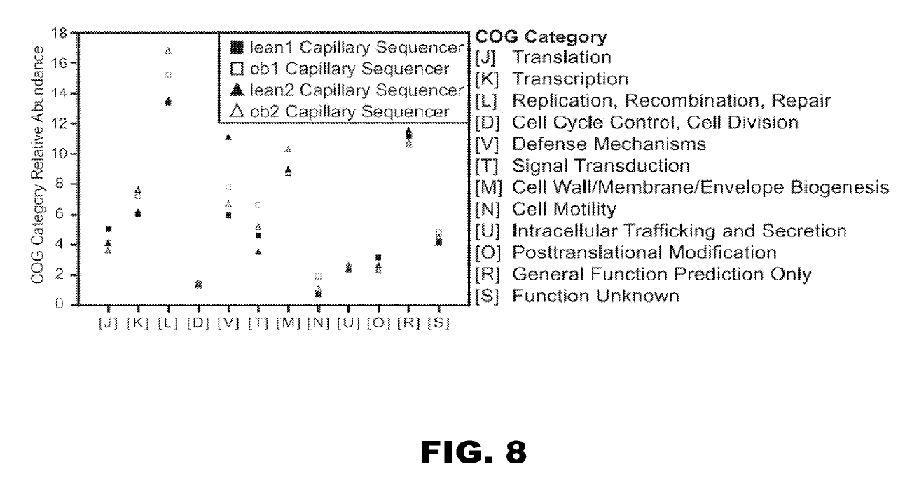

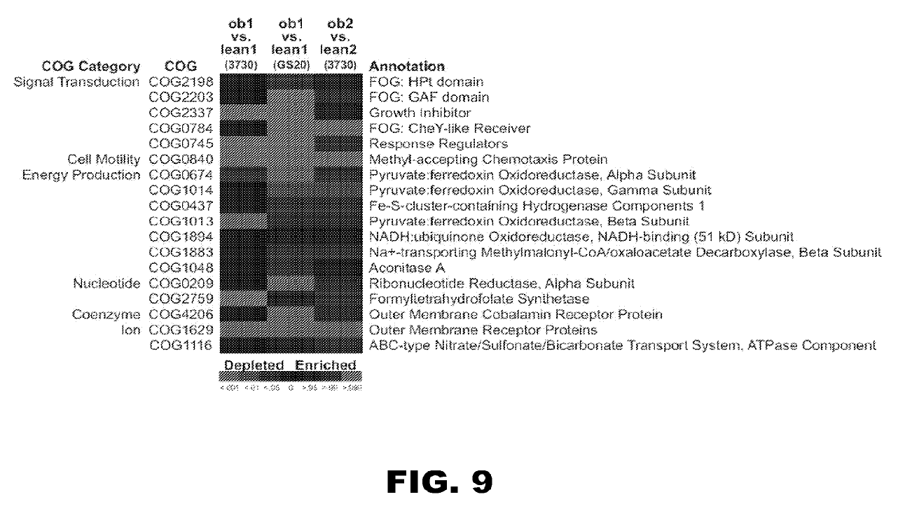

Results. Together, these data are consistent with an overall increase in the ability of the ob/ob microbiota to harvest energy from the diet. This notion was tested experimentally by performing microbiota transplantation experiments. Adult germ-free C57BL/6J mice were colonized (by gavage) with a microbiota harvested from the cecum of obese (ob/ob) or lean (+/+) donors (1 donor and 4-5 germ-free recipients per treatment group per experiment; two independent experiments). 16S rRNA gene sequence-based surveys confirmed that the ob/ob donor microbiota had a greater relative abundance of Firmicutes compared to the lean donor microbiota (FIG. 7; Table 7). Furthermore, the ob/ob recipient microbiota had a significantly higher relative abundance of Firmicutes compared to the lean recipient microbiota (p<0.05, two-tailed Student's t-Test). UniFrac analysis of 16S rRNA gene sequences obtained from the recipients' cecal microbiotas revealed that they cluster according to the input donor community (FIG. 7): i.e., the initial colonizing community structure did not exhibit marked changes by the end of the two-week experiment. There was no statistically significant difference in (i) chow consumption over the 14 day period [55.4±2.5 g (ob/ob) versus 54.0±1.2 g (+/+); caloric density of chow, 3.7 kcal/g], (ii) initial body fat (2.7±0.2 g for both groups as measured by dual energy x-ray absorptiometry; DEXA), or (iii) initial weight between the recipients of lean and obese microbiotas. Strikingly, mice colonized with an ob/ob microbiota exhibited a significantly greater percentage increase in body fat over two weeks than mice colonized with a +/+ microbiota [FIG. 6C; 47±8.3 vs. 27±3.6 percentage increase or 1.3±0.2 vs. 0.86±0.1 g fat (DEXA): at 9.3 kcal/g fat, this corresponds to a difference of 4 kcal or 2% of total calories consumed].

TABLE 7
16S rRNA gene-sequence libraries from microbiota transplant
experiments

| | | Host | 16S gene |
|---|---|---|---|
| Label in FIG. S4 | ARB label | Genotype | sequences |
| lean donor 1 | lean2 | +/+ | 166 |
| ob/ob donor 1 | obob1 | ob/ob | 199 |
| ob/ob donor 2 | obob2 | ob/ob | 229 |
| lean recipient 1 | SWPT11 | +/+ | 248 |
| lean recipient 2 | SWPT13 | +/+ | 265 |
| lean recipient 3 | SWPT18 | +/+ | 247 |
| lean recipient 4 | SWPT19 | +/+ | 278 |
| lean recipient 5 | SWPT20 | +/+ | 271 |
| ob/ob recipient 1 | SWPT1 | +/+ | 219 |
| ob/ob recipient 2 | SWPT2 | +/+ | 268 |
| ob/ob recipient 3 | SWPT3 | +/+ | 280 |
| ob/ob recipient 4 | SWPT4 | +/+ | 272 |
| ob/ob recipient 5 | SWPT5 | +/+ | 290 |
| ob/ob recipient 6 | SWPT12 | +/+ | 197 |
| ob/ob recipient 7 | SWPT14 | +/+ | 272 |
| ob/ob recipient 8 | SWPT15 | +/+ | 198 |
| ob/ob recipient 9 | SWPT16 | +/+ | 258 |
| TOTAL | — | — | 4,157 |

TABLE 8
KEGG pathways depleted in the pooled ob/ob cecal microbiome
relative to the pooled lean cecal microbiome (capillary sequencing
datasets, ob1 + ob2 vs. lean1 + lean2 + lean3, binomial
test, P < 0.05).

| KEGG Category | KEGG Pathway¹ |
|---|---|
| Carbohydrate Metabolism | Glycolysis/Gluconeogenesis |
| | Citrate cycle (TCA cycle) |
| | Pentose phosphate pathway |
| | Pentose and glucuronate interconversions |
| | Fructose and mannose metabolism |
| Energy Metabolism | Carbon fixation |
| | Reductive carboxylate cycle (CO2 fixation) |
| | Pyruvate/Oxoglutarate oxidoreductases |
| Lipid Metabolism | Fatty acid metabolism |
| Nucleotide Metabolism | Pyrimidine metabolism |
| Amino Acid Metabolism | Glutamate metabolism |
| | Glycine, serine and threonine metabolism |
| | Cysteine metabolism |
| | Arginine and proline metabolism |
| | Phenylalanine, tyrosine and tryptophan |
| | biosynthesis |
| Glycan Biosynthesis and | Lipopolysaccharide biosynthesis |
| Metabolism | |
| Metabolism of Cofactors | Riboflavin metabolism |
| and Vitamins | |
| | Folate biosynthesis |
| Translation | Ribosome |
| Folding, Sorting and | Other ion-coupled transporters |
| Degradation | |

¹Only pathways with greater than ten hits in both pooled datasets are shown.

TABLE 9
COG categories involved in information storage and cellular processes
that are enriched or depleted in the pooled ob/ob cecal microbiome
relative to the pooled lean cecal microbiome (capillary sequencing
datasets, ob1 + ob2 vs. lean1 + lean2 + lean3, binomial
test, P < 0.05).

ENRICHED
[K] Transcription
[L] Replication, recombination, repair
[Y] Nuclear structure
[T] Signal transduction
[M] Cell wall/membrane/envelope biogenesis
[N] Cell motility
DEPLETED
[J] Translation
[V] Defense mechanisms
[O] Posttranslational modification, protein turnover, chaperones

### Example 5

### Human Gut Microbes Linked to Obesity

Sequence generation and analysis. All subjects gave written informed consent before participating in this study, which was approved by the Washington University Human Studies Committee. We studied 12 men and women (21 to 65 years-old; body mass index (BMI) 30 to 43 kg/m2) who were randomly assigned to one of two low calorie diets: either a fat restricted (FAT-R; ˜30% of calories from fat) or a carbohydrate-restricted (GARB-R; ˜25% of calories from carbohydrates). The recommended caloric intake for women on either diet was 1200-1500 kcal/d, and 1500-1800 kcal/d for men. The total fiber content of both diets was similar (˜10-15 g/day). A morning stool sample was collected before and at 12, 26 and 52 weeks after starting diet therapy. Stool was also collected at 0 and 52 weeks from two healthy men (aged 32 and 36; BMI 23 kg/m2). DNA was extracted from morning stool specimens, and bacterial 16S rRNA gene sequences were generated with bacterial primers using protocols described in Ley et al. (2005) v102, pg. 11070-75, with the following modifications: (i) replicate PCR reaction mixtures were pooled, concentrated, purified using a Montage PCR cleanup kit (Millipore), and further purified (1% agarose gel electrophoresis) prior to cloning; (ii) three sequence reads were generated per cloned 16S rRNA gene amplicon using vector-specific primers and the internal primer 907R (see Ley et al., 2005 PNAS). 16S rRNA gene sequences were edited and assembled as outlined in Gell et al. Sequences were aligned using the nast online alignment tool (http://greengenes.lbl.gov/cgi-bin/nph-index.cgi), and checked for chimeras using Bellerophon (Huber (2004) Bioinformatics 20:2317-2319). Non-chimeric sequences >800 bp (n=18, 348) were added to an existing Arb alignment using the parsimony insertion tool (Ludwig (2004) Nucleic Acids Res 32:1363-71). Distance matrices, with Olsen correction, were generated in Arb. DOTUR was used (i) to cluster sequences >1 kb (n=16,177) into OTUs by % pair-wise identity (% ID, using a furthest-neighbor algorithm and a precision of 0.01), and (ii) to generate Shannon's diversity index (Schloss (2005) Appl. Env. Micro. 71:1501-6). We used UniFrac (Lozupone (2005) Appl. Env. Micro 71:8228-35) to cluster the samples based on an Arb-generated neighbor-joining tree. The alignment of the 18,348-sequence dataset is available at http://gordonlab.wustl.edu/microbial_ecology_human_obesity. Sequences have been deposited in GenBank under accession numbers DQ793220-DQ802819, DQ803048, DQ803139-DQ810181, DQ823640-825343.

Statistical analyses. Analysis of variance was conducted using a model comparison approach. The p-value associated with the correlation coefficient describing the relationship between the change in Bacteroidetes and the change in weight was generated by permutation analysis: values were scrambled randomly and a R2 generated 10,000 times; the distribution of R2 values was used to assess the probability of obtaining the observed R2.

Results. To explore the relationship between gut microbial ecology and body fat in humans, we studied 12 obese subjects randomly assigned to either a fat-restricted (FAT-R) or carbohydrate-restricted (CARB-R) low calorie diet. The composition of their gut microbiotas was monitored over one year by sequencing 16S rRNA genes from stool samples.

Using ≧97% sequence identity in 16S rRNA gene sequence among individuals as a definition of a species, the resulting dataset of 18,348 bacterial 16S rRNA sequences (Table 10) revealed that most (70%) of the 4,074 identified species-level phylogenetic types (phylotypes) were unique to individual subjects. Despite the marked interpersonal differences in species-level diversity, members of the Bacteroidetes and Firmicutes divisions dominated the microbiota (92.6% of all 16S rRNA sequences).

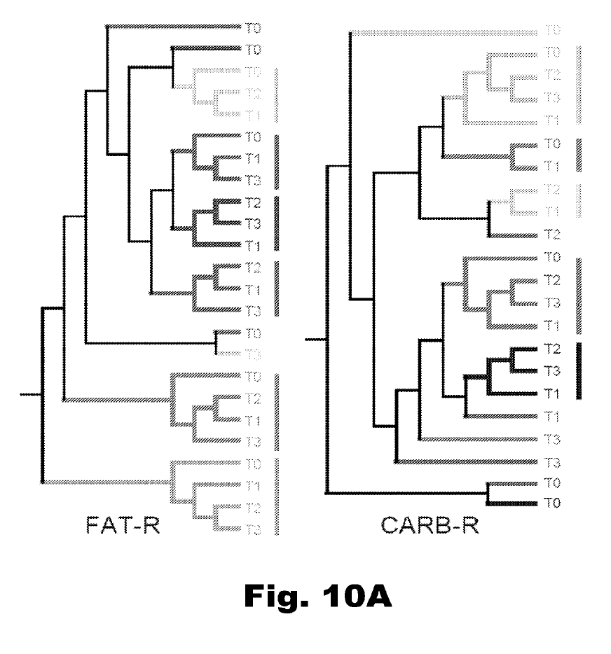

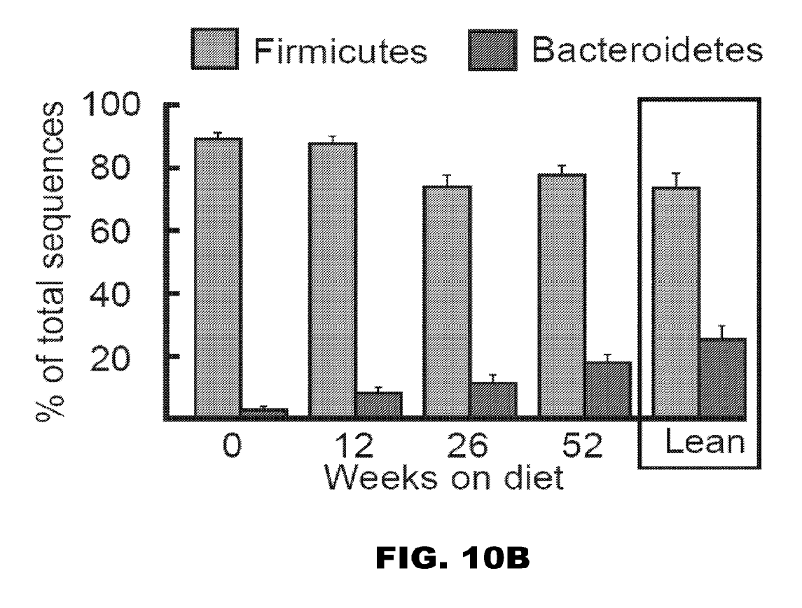

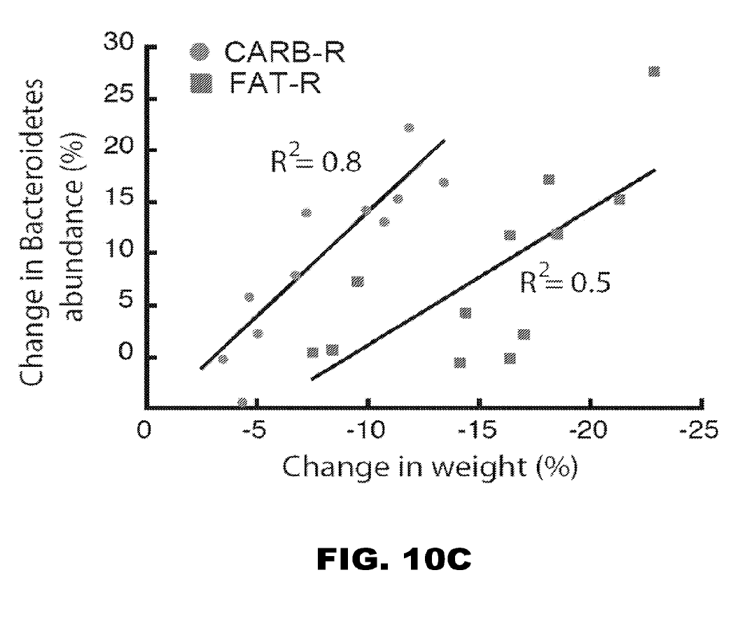

Bacterial lineages were remarkably constant within subjects over time: communities from the same subject were generally more similar to one another than to communities from other subjects (FIG. 10A). Before diet therapy, obese subjects had fewer Bacteroidetes (p<0.001) and more Firmicutes (p=0.002) than lean controls (FIG. 10B). Over time, the relative abundance of Bacteroidetes increased (% Bacteroidetes vs. weeks, p<0.001) and the abundance of Firmicutes decreased (% Firmicutes vs. weeks, p=0.002), irrespective of the type of diet (FIG. 10B). Remarkably, this change was division-wide, and not due to blooms or extinctions of specific bacterial species. Correspondingly, diversity levels were constant over time. The increased Bacteroidetes abundance correlated directly with percent weight loss (R2=0.8 and 0.5 for the CARB-R and FAT-R diets, respectively; p<0.05; FIG. 10C), and not with changes in calorie content over time (R²=0.06 and 0.09 for the CARB-R and FAT-R diets, respectively). The correlation between Bacteroidetes abundance and weight loss was observed only after a threshold weight loss of 6% for FAT-R and 2% for CARB-R was attained.

Obesity is the only disease process that we are aware of where a pronounced, division-wide change in microbial ecology has been associated with host pathology. As such, it represents an attractive model for studying the role of the microbiota in health and disease. The factors that drive shifts in representation at such broad taxonomic levels must operate on highly conserved bacterial traits since they are shared by a great variety of phylotypes within the divisions. The gut habitat itself selects for specific ratios of divisions: microbiotas transplanted from a donor species to germ-free recipients of a different species reconfigure to match the community structure normally occurring in the recipient. The coexistence of Bacteroidetes and Firmicutes in the gut implies minimized competition for resources by cooperation or specialization: the obese gut possesses yet uncharacterized physical or chemical properties that tip the balance towards the Firmicutes.

The direct correlation between the abundance of the Bacteroidetes and the amount of weight loss in obese subjects reveals a dynamic linkage between adiposity and gut microbial ecology. These findings, together with results obtained from mice, suggest that intentional manipulation of gut microbial communities could be a new approach for treating obesity.

TABLE 10
Sequence prefixes by library, and the number of sequences per library (N)

| | | | | 0 | | 12 | | 26 | | 52 | |
|---|---|---|---|---|---|---|---|---|---|---|---|
| | | | | weeks | | weeks | | weeks | | weeks | |
| | | | | Library | | Library | | Library | | Library | |
| Subject | Sex | Age | Diet Group | prefix | N | prefix | N | prefix | N | prefix | N |
| 1 | F | 57 | FAT-R | RL178 | 541 | RL240 | 327 | RL197 | 202 | RL310 | 328 |
| 2 | F | 53 | FAT-R | RL182 | 178 | RL242 | 296 | RL205 | 277 | RL305 | 346 |
| 3 | F | 54 | FAT-R | RL187 | 803 | RL251 | 287 | RL200 | 335 | RL385 | 274 |
| 4 | F | 48 | FAT-R | RL188 | 579 | RL241 | 287 | RL201 | 310 | RL311 | 244 |
| 5 | M | 55 | FAT-R | RL180 | 855 | RL244 | 312 | RL198 | 189 | RL307 | 309 |
| 6 | M | 55 | FAT-R | RL184 | 877 | RL243 | 306 | RL239 | 289 | RL308 | 235 |
| 7 | F | 42 | CARB-R | RL176 | 543 | RL246 | 236 | RL199 | 309 | | |
| 8 | F | 30 | CARB-R | RL179 | 767 | RL245 | 215 | RL202 | 271 | RL386 | 294 |
| 9 | F | 42 | CARB-R | RL181 | 539 | RL248 | 302 | RL206 | 325 | RL302 | 337 |
| 10 | F | 49 | CARB-R | RL183 | 481 | RL247 | 309 | | | RL303 | 254 |
| 11 | F | 35 | CARB-R | RL186 | 865 | RL249 | 227 | RL203 | 304 | RL306 | 331 |
| 12 | M | 54 | CARB-R | RL185 | 831 | RL250 | 284 | RL204 | 290 | RL304 | 300 |
| 13 | M | 32 | CONTROL | RL116 | 100 | | | | | RL387 | 252 |
| 14 | M | 36 | CONTROL | RL117 | 93 | | | | | RL388 | 303 |
| | | | | | | | | | | TOTAL | 18,348 |

### Example 6

### Diet-Induced Obesity Alters Gut Microbial Ecology

The following materials and methods are also applicable to examples 7 and 8.

Animals—All experiments involving mice were performed using protocols approved by the Washington University Animal Studies Committee.

Conventionalization—Germ-free male 8-9 week old C57BL/6J mice were maintained in plastic gnotobiotic isolators, under a strict 12-h light cycle and fed an autoclaved low-fat, polysaccharide-rich chow diet (CHO) ad libitum [1,28]. Conventionalization was performed by harvesting cecal contents from conventionally-raised animals, and introducing them, by gavage, into germ-free recipients, as described in ref. 4.

Conventionally-raised mice—Once C57BL/6J littermates were weaned, they were housed individually in microisolator cages where they were maintained in a specified pathogen-free state, under a 12-h light cycle, and fed a CHO diet (PicoLab, Purina), a high-fat/high-sugar Western diet (Harlan-Teklad TD96132), a fat-restricted (FAT-R) diet (Harlan-Teklad TD05633), or a carbohydrate-restricted (CARB-R) diet (Harlan-Teklad TD05634) ad libitum.

Microbiota transplantation experiments—Adult germ-free C57BL/6J mice 8 weeks old were colonized with a cecal microbiota obtained from wild-type (+/+) C57BL/6J donor mice fed CHO, Western, FAT-R, or CARB-R diets. Recipient mice, maintained on a CHO diet, were anesthetized at 0.5 and 14 days post colonization with an intraperitoneal injection of ketamine (10 mg/kg body weight) and xylazine (10 mg/kg) and total body fat content was measured by dual-energy x-ray absorptiometry (DEXA; Lunar PIXImus Mouse, GE Medical Systems) [29]. Recipient mice were housed individually in microisolator cages within gnotobiotic isolators throughout the experiment to avoid exposure to the microbiota of the other mice, and to allow the direct monitoring of the chow consumed by each mouse. Animals were sacrificed immediately after the final DEXA on day 14.

Shotgun sequencing and assembly of cecal microbiomes—DNA samples were used to construct pOTw13-based libraries (GC10 cells, Gene Choice) for capillary-based sequencing with an ABI 3730x1 instrument. Unidirectional (forward) sequencing reads were generated from each library (an average of 10,600 reads/library). Reverse reads were also generated to improve assembly (768-1536 per library; total of 7,680 reads;). Sequences were trimmed based on quality score and vector sequences were removed prior to analysis (Applied Biosystems; KB Basecaller). Each dataset was assembled individually, in addition to a combined assembly of all seven datasets, using ARACHNE (parameters: maxcliq1=500; maxcliq2=500; genome size=1 Gb) [24]. ARACHNE was chosen because it has been shown to generate reliable contigs from complex simulated metagenomic datasets [30]. Genes were predicted from individual sequencing reads and contigs using MetaGene [25].

Microbiome functional analysis—NCBI BLAST was used to query the STRING-extended COG database [19] and the KEGG database (version 40) [20]. COG and KEGG comparisons were performed by using NCBI BLASTX employing default parameters. A cutoff of e-value <10⁻⁵ was used for environmental gene tag (EGT) assignments and sequence comparisons. Predicted proteins were searched for conserved domains and assigned functional identifiers with InterProScan (version 4.3) [31]. Predicted glycoside hydrolases were confirmed based on criteria used for the Carbohydrate Active Enzymes (CAZy) database (http://www.cazy.org/; Bernard Henrissat, personal communication).

Statistical methods—X² tests were performed on the number of gene assignments to a given KEGG or STRING orthologous group in each microbiome relative to the number of gene assignments to all other groups. Xipe (version 2.4) [32] was employed for bootstrap analyses of KEGG pathway enrichment and depletion, as described previously [2], using the parameters sample size=10,000 and confidence level=0.90. ANOVA was performed using a model comparison approach [33], implemented with the linear regression function in Excel (version 11.0, Microsoft). Student's t-tests were utilized to identify statistically significant differences between two groups. Data are represented as mean±SEM unless otherwise indicated. The p-value associated with a given correlation coefficient (R²) was generated by a permutation analysis, as described previously [9]. Briefly, the values were scrambled randomly and an R² generated 10,000 times; the resulting distribution of R² values was used to assess the probability of obtaining the observed R².

Preparation of DNA from the Cecal Microbiota—Cecal contents were frozen at −80° C. immediately after sacrifice. An aliquot (˜10 mg) of each sample was then suspended, while frozen, in a solution containing 500 μl of extraction buffer [200 mM Tris (pH 8.0), 200 mM NaCl, 20 mM EDTA], 210 μl of 20% SDS, 500 μl of a mixture of phenol:chloroform:isoamyl alcohol (pH 7.9, 25:24:1), and 500 μl of a slurry of 0.1 mm-diameter zirconia/silica beads (BioSpec Products, Bartlesville, Okla.). Microbial cells were subsequently lysed by mechanical disruption with a bead beater (BioSpec Products) set on high for 2 min at RT, followed by extraction with phenol:chloroform:isoamyl alcohol (pH 7.9, 25:24:1), and precipitation with isopropanol. DNA obtained from ten separate 10 mg frozen aliquots of each cecal sample were pooled (≧200 μg DNA) and used to construct plasmid libraries (pOTw13) for 3730x1 capillary-based metagenomic sequencing (see below).

16S rRNA sequence-based surveys of the distal gut (cecal) mouse microbiota—Five replicate PCR reactions were performed for each cecal DNA sample. Each 25 μl reaction contained 50-100 ng of purified DNA, 10 mM Tris (pH 8.3), 50 mM KCl, 2 mM MgSO₄, 0.16 μM dNTPs, 0.4 μM of the bacteria-specific primer 8F (5′-AGAGTTTGATCCTGGCTCAG-3′), 0.4 μM of the universal primer 1391R (5′-GACGGGCGGTGWGTRCA-3′), 0.4 M betaine, and 3 units of Taq polymerase (Invitrogen). Cycling conditions were 94° C. for 2 min, followed by 35 cycles of 94° C. for 1 min, 55° C. for 45 sec, and 72° C. for 2 min, with a final extension period of 20 min at 72° C. Replicate PCRs were pooled and concentrated (Millipore; Montage PCR filter columns). Full-length 16S rRNA gene amplicons (1.3 kb) were then gel-purified using the Qiaquick kit (Qiagen), subcloned into TOPO TA pCR4.0 (Invitrogen), and the ligated DNA transformed into E. coli TOP10 (Invitrogen). For each mouse, 384 colonies containing cloned amplicons were processed for sequencing. Plasmid inserts were sequenced bi-directionally using vector-specific primers plus the internal primer 907R (5′-CCGTCAATTCCTTTRAGTTT-3′).

16S rRNA gene sequences were edited and assembled into consensus sequences using the PHRED and PHRAP software packages within the Xplorseq program [39]. Sequences that did not assemble were discarded and bases with PHRED quality scores <20 were trimmed. Sequences were checked for chimeras using Bellerophon version 2 [40] and sequences with greater than 95% identity to both parents were removed (n=535; 13% of aligned sequences). The final dataset (n=8,511 16S rRNA gene sequences; for sequence designations see Table 13) was aligned using the on-line version of the NAST multi-aligner [41] [minimum alignment length=1250 nucleotides (500 for Rag1−/− data); percent identity >75]. Hypervariable regions were masked using the lanemaskPH filter provided within the ARB database [42], and the aligned sequences added to the ARB neighbor-joining tree (based on pairwise distances with the Olsen correction), using the parsimony insertion tool. A phylogenetic tree containing all 16S rRNA gene sequences was then exported from ARB, clustered using online UniFrac [12] without abundance weighting, and visualized with TreeView [43]. A distance matrix of all 16S rRNA gene sequences was imported into DOTUR [13] for phylotype binning and measurements of diversity (e.g., the Shannon index).

Taxonomic assignment of shotgun sequencing reads—Quality-trimmed reads were assigned to reference genomes by comparison with the NCBI non-redundant database (NR version Apr. 19, 2007; BLASTX e-value <10⁻⁵; BLASTX parameters ‘-F F’). Sequences were assigned to the taxonomic group (division, class, genus, etc.) that would include all significant hits using MEGAN (under the default parameters, only reads with a BLAST score 0% of the top score were included) [18]. Reads containing a 16S rRNA fragment were identified by BLASTN comparison of each microbiome to the RDP database (version 9.33) [34]. 16S rRNA gene fragments were then aligned using the NASTA multi-aligner [41] with a minimum template length of 400 bases and a minimum percent identity of 75%. The resulting alignment was then imported into an ARB neighbor-joining tree and hypervariable regions masked using the lanemaskPH filter [42].

Transcriptional profiling—A 10 mg aliquot of frozen cecal contents from a mouse fed the Western diet (sample ‘Western 3’) was immersed in 1 ml of RNAProtect (Qiagen), vortexed, centrifuged for 10 min at 5000×g, and the supernatant was removed. Microbial cells in the pellet were subsequently lysed by mechanical disruption with a bead beater (BioSpec Products) set on high for 2 min at RT in a solution containing 500 μl of extraction buffer. RNA was extracted with phenol:chloroform:isoamyl alcohol (pH 4.5, 125:24:1), precipitated with isopropanol, and further purified with (i) the RNeasy Mini Kit (Qiagen), (ii) on-column digestion with DNAseI (Qiagen), (iii) an additional DNAse treatment (DNAfree kit, Ambion), and (iv) passage through a RNeasy column (Qiagen).

A modification of the protocol included with the MessageAmpII-bacteria Kit (Ambion) that was developed at MIT [44], was used for mRNA-enriched cDNA synthesis. cDNA was purified (Qiaquick, Qiagen) and subcloned into pSMART (10G Supreme Cells, Lucigen). Plasmid inserts from 384 randomly picked colonies were sequenced (single unidirectional reads) using vector specific primers and an ABI 3730x1 instrument. Sequences were trimmed based on quality score and to remove vector sequences (Applied Biosystems; KB Basecaller), and to remove poly(A) tails. Only sequences with a final length ≧180 bases were analyzed (average of 430 nucleotides). Sequences were annotated based on BLASTX (see above) and BLASTN comparisons against the NCBI nucleotide database (version Sep. 26, 2007; BLASTN parameters ‘−F F’). 16S rRNA sequences were annotated based on their best-BLAST hit to 16S rRNA genes of known taxonomic origin (e-value <10−25). Although rRNA gene fragments were the dominant sequence (90.6% of the high-quality reads), the library had a lower abundance of rRNA transcripts than comparable libraries created directly from total distal gut community RNA (99%; P. J. Turnbaugh and J. I. Gordon, unpublished data).

### Results

The leptin deficient, ob/ob mouse model of obesity established a correlation between host adiposity, microbial community structure, and the efficiency of energy extraction from a standard, low-fat rodent chow diet that was rich in plant polysaccharides, but it did not allow us to investigate the effects of manipulating diet, or diminishing host adiposity on the gut microbiota and its microbiome. Furthermore, leptin deficiency is extremely rare in humans and is associated with a variety of other host phenotypes [11]. Therefore, the following examples, turns to a mouse model of diet-induced obesity (DIO) produced by consumption of a prototypic high-fat/high-sugar Western diet, where all animals were genetically identical, ‘inherited’ a similar microbiota, and where once an obese state was achieved, specified diets could be imposed to reduce adiposity.

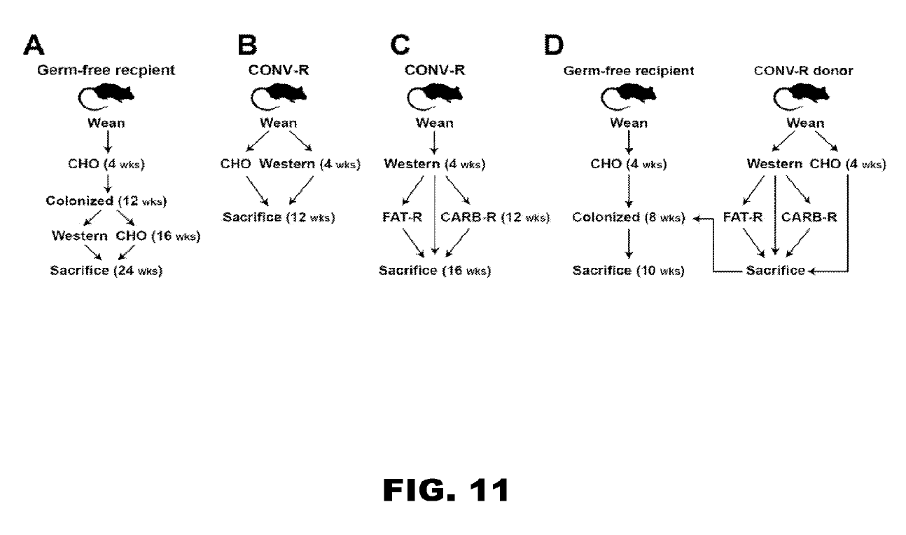

Ten germ-free male C57BL/6J mice were weaned onto a low-fat chow diet rich in structurally complex plant polysaccharides (‘CHO’ diet), and then gavaged at 12 weeks of age with a distal gut (cecal) microbiota harvested from a conventionally-raised donor (see Table 11 for the percentage of calories derived from protein, carbohydrate, and fat). This process of ‘conventionalization’ was designed to insure that all recipients inherited a similar microbiota. All recipients were subsequently maintained in gnotobiotic isolators. Four weeks later, five of the conventionalized mice were switched to ‘Western’ diet high in saturated and unsaturated fats (41% of total calories) and the types of carbohydrates commonly used as human food additives [sucrose (18% of chow weight), maltodextrin (12%), plus corn starch (16%); Tables 11 and 12]. The remaining five mice were continued on the CHO diet. All mice were sacrificed eight weeks later (24 weeks after birth) (FIG. 11A). Mice on the Western diet gained significantly more weight than mice maintained on the CHO diet (5.3±0.8 g versus 1.5±0.2 g; p<0.05, Student's t-test) and had significantly more epididymal fat (3.7±0.5% versus 1.7±0.1% of total body weight; p<0.01, Student's t-test).

TABLE 11
Protein, carbohydrate, and fat composition of various mouse chow diets

| Diet | Protein^{a} | CHO^{a} | Fat^{a} | kcal/g |
|---|---|---|---|---|
| CHO^{b} | 23.2 | 60.7 | 16.1 | 3.74 |
| Western | 18.7 | 40.7 | 40.7 | 4.49 |
| FAT-R | 18.7 | 60.0 | 21.3 | 3.95 |
| CARB-R | 48.3 | 11.2 | 40.5 | 4.31 |

^{a}values represent percentage of total kcal;
^{b}B&K Universal autoclavable chow diet (Sonnenburg et al., (2006) PloS Biol 4: e413)

TABLE 12
Percent weight of chow ingredients

| Ingredient | Western | FAT-R | CARB-R |
|---|---|---|---|
| Casein | 23.6 | 20.8 | 59.9 |
| DL-Methionine | 0.354 | 0.354 | 0.000 |
| Sucrose, Cane | 18.3 | 32.0 | 0.000 |
| Corn Starch | 16.0 | 16.0 | 0.000 |
| Maltodextrin(Lo-Dex) | 12.0 | 12.0 | 11.0 |
| Vegetable Oil | 10.0 | 5.00 | 10.0 |
| Beef Tallow | 10.0 | 4.10 | 8.80 |
| Cellulose(Fiber) | 4.00 | 4.00 | 4.00 |
| Mineral Mix | 4.13 | 4.13 | 4.13 |
| (AIN-93G-MX) | | | |
| Calcium Phosphate Dibasic | 0.472 | 0.472 | 0.472 |
| Vitamin Mix | 1.18 | 1.18 | 1.18 |
| (Teklad 40060) | | | |
| Ethoxyquin(Antioxidant) | 0.002 | 0.002 | 0.002 |
| Calcium Carbonate | 0.000 | 0.000 | 0.500 |

Cecal microbial community structure was defined in each mouse in each of the two groups by sequencing full-length 16S rRNA gene amplicons produced by PCR of community DNA (see Materials and Methods in Supporting Information; n=96-343 16S rRNA gene sequences defined per mouse; Table 13). Communities were then compared using the UniFrac metric [12]. The premise of UniFrac is that two microbial communities with a shared evolutionary history will share branches on a 16S rRNA phylogenetic tree, and that the fraction of branch length shared can be quantified and interpreted as the degree of community similarity.

TABLE 13
16S rRNA gene-sequence libraries

| | | | | 16S |
|---|---|---|---|---|
| | | | | gene |
| | ARB | | Host | se- |
| Figure label | label | Host | diet | quences |
| CARB 1 | WD1 | CONV-D wt | CHO | 96 |
| CARB 2 | WD2 | CONV-D wt | CHO | 343 |
| CARB 3 | WD3 | CONV-D wt | CHO | 267 |
| CARB 4 | WD4 | CONV-D wt | CHO | 207 |
| CARB 5 | WD5 | CONV-D wt | CHO | 216 |
| Western 1 | WD6 | CONV-D wt | Western | 222 |
| Western 2 | WD7 | CONV-D wt | Western | 256 |
| Western 3 | WD8 | CONV-D wt | Western | 221 |
| Western 4 | WD9 | CONV-D wt | Western | 220 |
| Western 5 | WD10 | CONV-D wt | Western | 221 |
| Donor 1 | WD11 | CONV-R wt | — | 194 |
| CARB-R 1 | MD4 | CONV-R wt DIO, family 2 | CARB- | 185 |
| | | | R | |
| CARB-R 2 | MD8 | CONV-R wt DIO, family 1 | CARB- | 233 |
| | | | R | |
| CARB-R 3 | MD9 | CONV-R wt DIO, family 1 | CARB- | 184 |
| | | | R | |
| CARB-R 4 | MD21 | CONV-R wt DIO, family 2 | CARB- | 259 |
| | | | R | |
| CARB-R 5 | MD23 | CONV-R wt DIO, family 1 | CARB- | 516 |
| | | | R | |
| CARB-R 6 | MD26 | CONV-R wt DIO, family 2 | CARB- | 138 |
| | | | R | |
| FAT-R 1 | MD18 | CONV-R wt DIO, family 2 | FAT-R | 241 |
| FAT-R 2 | MD19 | CONV-R wt DIO, family 2 | FAT-R | 203 |
| FAT-R 3 | MD24 | CONV-R wt DIO, family 2 | FAT-R | 177 |
| FAT-R 4 | MD25 | CONV-R wt DIO, family 2 | FAT-R | 162 |
| FAT-R 5 | MD27 | CONV-R wt DIO, family 2 | FAT-R | 127 |
| Western 6 | MD2 | CONV-R wt DIO, family 2 | Western | 263 |
| Western 7 | MD6 | CONV-R wt DIO, family 2 | Western | 126 |
| Western 8 | MD7 | CONV-R wt DIO, family 2 | Western | 176 |
| Western 9 | MD20 | CONV-R wt DIO, family 2 | Western | 233 |
| Western 10 | MD22 | CONV-R wt DIO, family 2 | Western | 193 |
| CARB 6 | myd1 | CONV-R MyD88 −/− | CHO | 241 |
| CARB 7 | myd2 | CONV-R MyD88 −/− | CHO | 260 |
| CARB 8 | myd3 | CONV-R MyD88 −/− | CHO | 266 |
| Western 11 | myd4 | CONV-R MyD88 −/− | Western | 223 |
| Western 12 | myd5 | CONV-R MyD88 −/− | Western | 231 |
| — | rag2 | CONV-R Rag1 −/− | CHO | 66 |
| — | rag3 | CONV-R Rag1 −/− | CHO | 103 |
| — | rag4 | CONV-R Rag1 −/− | Western | 84 |
| — | rag5 | CONV-R Rag1 −/− | Western | 111 |
| | rag6 | CONV-R Rag1 −/− | Western | 94 |
| — | CRWD2 | CONV-R wt | Western | 272 |
| — | CRWD4 | CONV-R wt | CHO | 265 |
| — | CRWD5 | CONV-R wt | CHO | 167 |
| — | CRWD6 | CONV-R wt | Western | 225 |

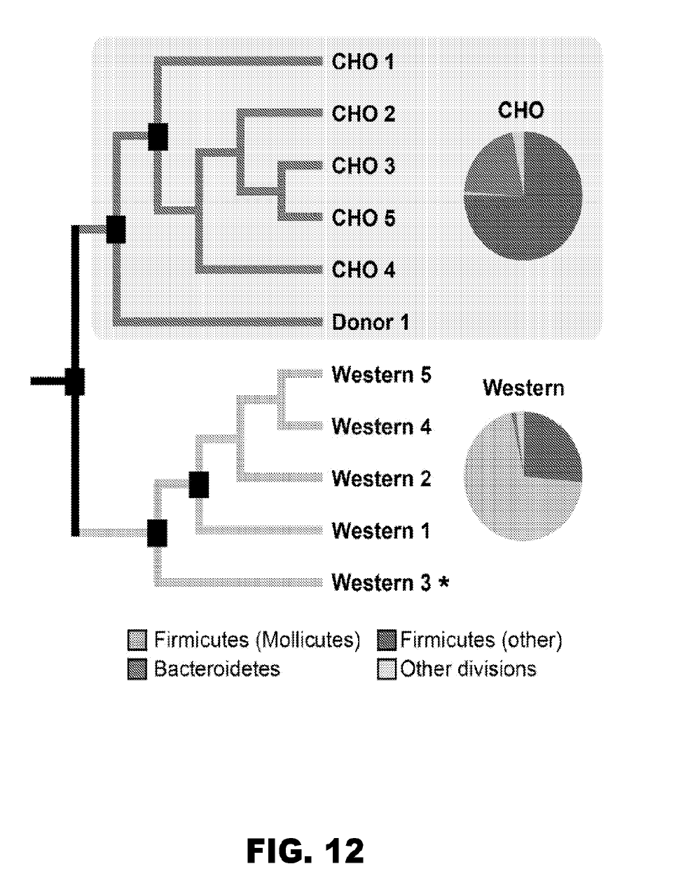

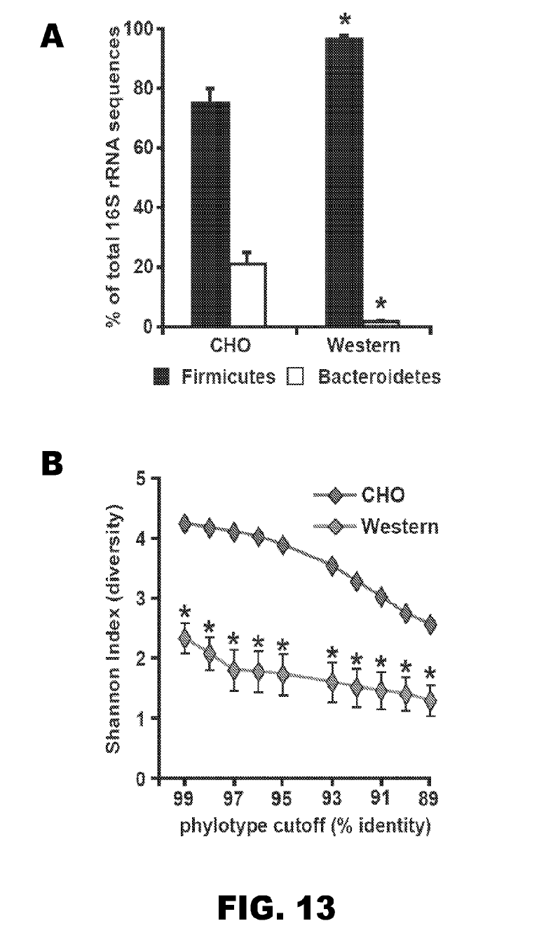

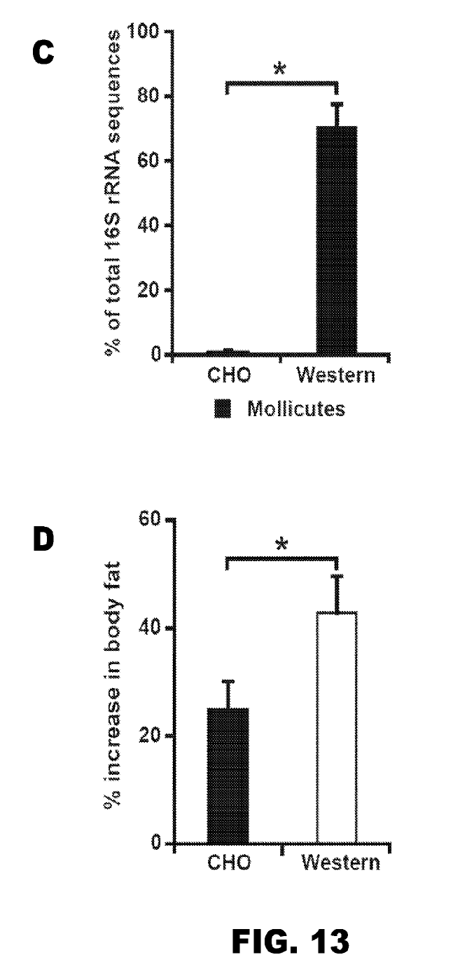

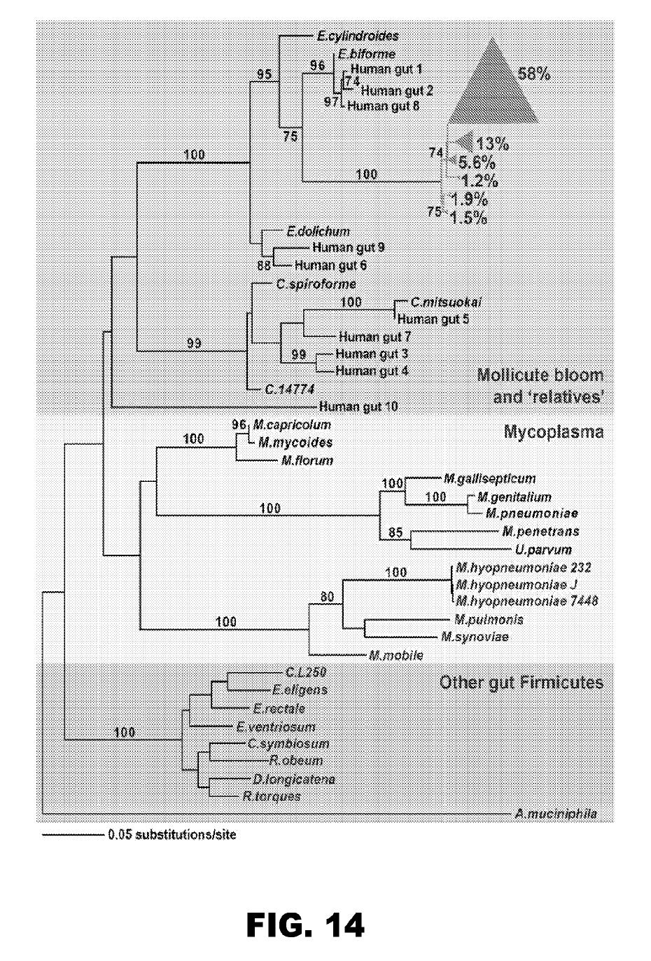

The results of UniFrac analysis revealed that the five Western diet-associated cecal communities were more similar to each other than to the five lean gut communities (FIG. 12). As in the ob/ob model of obesity, the Western diet-associated cecal community had a significantly higher relative abundance of the Firmicutes and a significantly lower relative abundance of the Bacteroidetes (FIG. 13A). Unlike the ob/ob microbiota, the observed shift in the Firmicutes was not division-wide: the overall diversity of the Western diet microbiota dropped dramatically, due to a bloom in a single class of the Firmicutes—the Mollicutes (FIGS. 13B,C and 12). Using 99% sequence identity among 16S rRNA genes as a threshold cutoff, we identified 132 ‘strain’-level phylotypes represented within the Mollicute bloom: the bloom was dominated by six phylotypes that together comprised 81% of the Mollicute sequences (FIG. 14) [13]. Other Mollicutes phylogenetically related to this Glade have been cultured from the human gut (e.g. Eubacterium dolichum, E. cylindroides, and E. biforme) and observed in 16S rRNA datasets generated from the fecal microbiota of obese humans [9]. However, there are no reported cultured representatives of the dominant phylotypes observed in the DIO mouse model (FIG. 14).

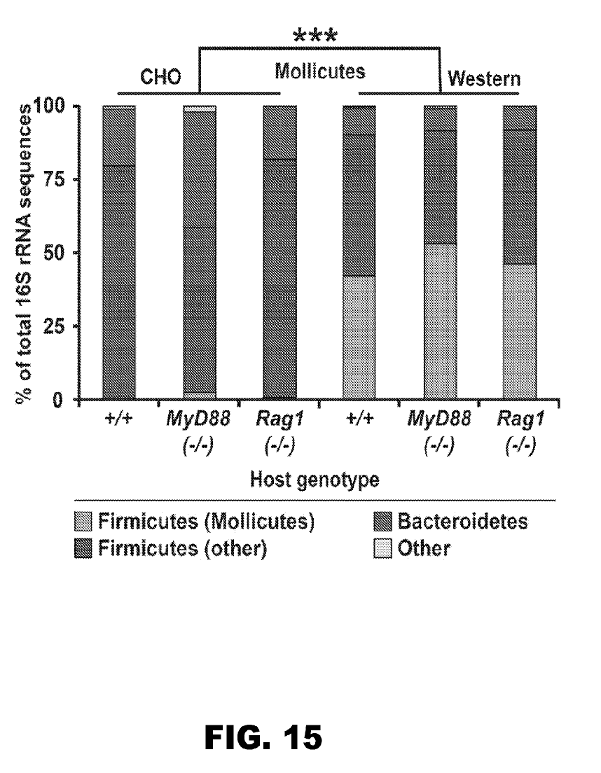

To determine whether these diet-induced changes in gut microbial ecology also occur in mice exposed to microbes starting at birth, we conducted a follow-up study using a different experimental design. In this case, conventionally-raised C57BL/6J mice were weaned onto a Western or a CHO diet and then maintained, in separate cages, on those diets for 8-9 weeks (n=8 animals/group). All animals were sacrificed after 12 weeks of age (FIG. 11B). Those on the Western diet gained significantly more weight (13.8±0.9 g versus 10.9±0.9 g; p<0.05, Student's t-test) and had significantly greater adiposity (epididymal fat pad weight was 3.0±0.2% of total body weight in the Western diet group versus 1.6±0.1% in the CHO group; p<0.001, Student's t-test). The cecal microbiota of these conventionally-raised mice fed the Western diet was dominated by the same Mollicute lineage that had been identified in the earlier conventionalization experiment involving germ-free animals (FIG. 15).

The immune system is one of the host factors that influences gut microbial ecology [14-17]. However, this bloom occurred in all mice fed the Western diet and did not require a functional innate or adaptive immune system: i.e., the Mollicute bloom was present at a significantly higher abundance in the cecal microbiota of conventionally-raised Western diet-fed C57BL/6J mice that were wild-type, MyD88−/− or Rag1−/−, compared to their genotypically-matched CHO-fed siblings (FIG. 15).

To directly test whether the DIO-associated gut microbial community possesses functional attributes that can increase host adiposity to a greater degree than a CHO-diet associated gut microbial community, we transplanted the cecal microbiota harvested from obese, conventionally-raised wild-type donors who had been on the Western diet for 8 weeks since weaning, or the cecal microbiota from lean CHO-fed controls, to 8-9 week-old germ-free CHO-fed recipients (n=1 donor and 4-5 recipients/treatment group/experiment; n=3 independent experiments, including one CHO-fed control group described in ref. 2). All recipients were maintained on a CHO diet (16% of kcal from fat, 61% from carbohydrates of which 2% are from fructose, glucose, lactose, maltose, and sucrose combined), and sacrificed 14d after receiving the microbiota transplant (FIG. 11D). Mice colonized with a DIO-associated microbiota exhibited a significantly greater percentage increase in body fat, as defined by dual energy x-ray absorptiometry (DEXA), than mice who had been gavaged with a microbiota from CHO-fed donors (43.0±7.1 versus 24.8±4.9 percentage increase; p<0.05, Student's t-test based on the combined data from all three experiments) (FIG. 13D). Importantly, there were no statistically significant differences in chow consumption (14.5±0.3 versus 14.7±0.8 kcal/d) or initial weight (22.9±0.3 versus 23.8±0.7 g) between recipients of the obese and lean cecal microbiotas.

To test the impact of defined shifts in diet on the body weight, adiposity, and distal gut microbial ecology of obese mice, we designed two custom chows that were modifications of the Western diet: one with reduced carbohydrates (CARB-R); the other with reduced fat (FAT-R) (see Tables 11 and 12 for information about the composition and caloric density of these diets). Sixteen conventionally-raised C57BL/6J mice, representing two families derived from two mothers who were sisters to ensure that they all inherited a similar microbial community [8], were weaned onto the Western diet and maintained on it for two months. A subset of mice from each family was subsequently continued on the Western diet for four weeks (n=5; control group), while the remaining siblings were switched to the CARB-R (n=6), or FAT-R diets (n=5) for four weeks (FIG. 11C).

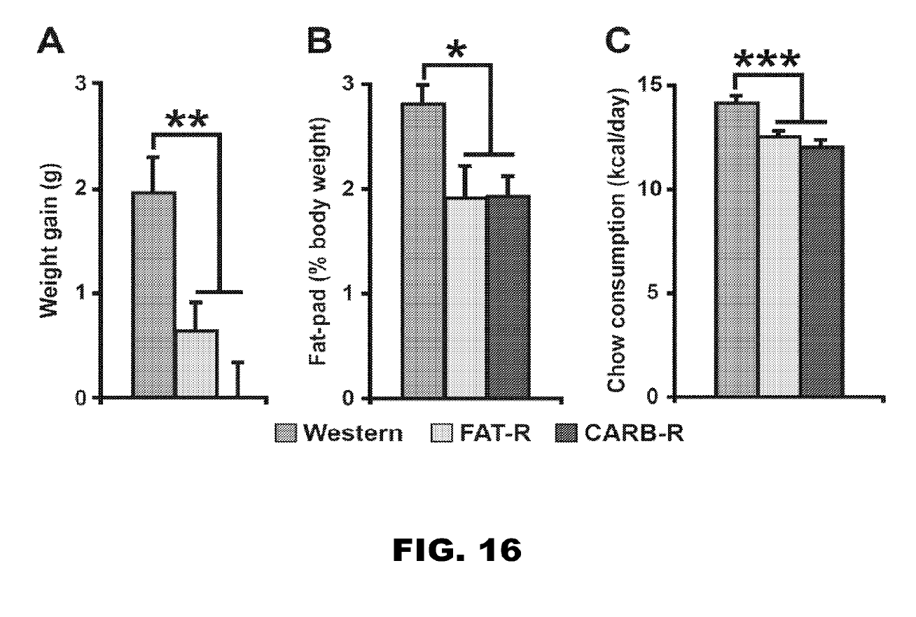

Mice switched to the FAT-R or CARB-R diet consumed significantly fewer calories [12.5±0.1 kcal/d (FAT-R) and 12.0±0.2 kcal/d (CARB-R) versus 14.1±0.2 kcal/d (Western); p<0.0001, ANOVA], gained significantly less weight [0.6±0.3 g (FAT-R) and 0.0±0.3 g (CARB-R) versus 2.0±0.3 g (Western); p<0.01, ANOVA], and had significantly less fat [epididymal fat pad weight: 1.9±0.3% of total body weight for FAT-R and 1.9±0.2% for CARB-R versus 2.8±0.2% (Western); p<0.05, ANOVA] than those maintained on the Western diet (FIG. 16). This provided us with the animal model we had sought: diet-induced obesity followed by weight stabilization and reductions in adiposity, in genetically identical mice consuming defined diets who had inherited a similar microbiota from their mothers.

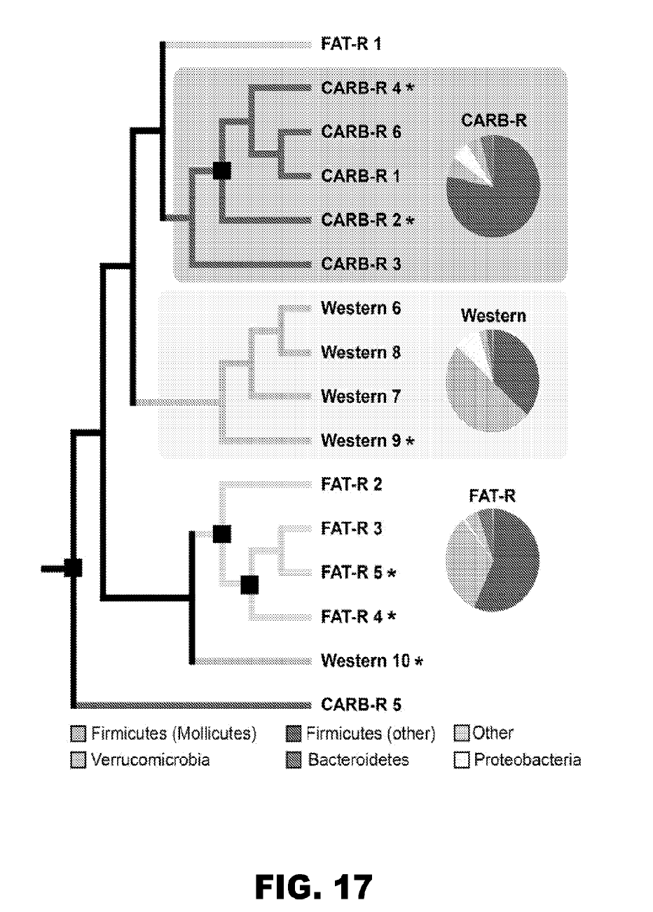

16S rRNA gene sequence-based surveys revealed that weight stabilization was accompanied by (i) a significant reduction in the relative abundance of the Mollicutes [31.9±11.6% of all bacterial sequences for FAT-R, and a significantly more pronounced decrease to 6.1±3.6% for CARB-R versus 50.3±6.1% for the Western diet; p<0.05, ANOVA], and (ii) a significant division-wide increase in the relative abundance of Bacteroidetes (2.8-fold on the FAT-R, and 2.2-fold on the CARB-R diets; p<0.05, ANOVA) (FIG. 17).

To test if these alterations in gut microbial ecology had an effect on the ability of the microbiota to promote host adiposity, we colonized germ-free, CHO-fed recipients with a cecal microbiota harvested from conventionally-raised donors who had been on the Western diet since weaning (8 weeks) and then switched to a FAT-R or CARB-R diet (n=1 donor and 4-5 germ-free recipients/treatment group/experiment; n=2 independent experiments; FIG. 11D). Unlike with recipients of the DIO-associated microbiota, there was no statistically significant difference in the amount of fat gained between mice colonized with the FAT-R or CARB-R communities, compared to mice colonized with a cecal microbiota from lean CHO-fed donors (33.6±8.7%, 37.4±10.6%, and 24.8±4.9% increases, respectively; p=0.2, ANOVA).

Combined, these results indicate that both the FAT-R and CARB-R diets repress multiple effects of Western diet-induced obesity: i.e. they decrease adipose tissue mass, diminish the bloom in a single uncultured Mollicute lineage, increase the relative abundance of Bacteroidetes, and reduce the ability of the microbiota to promote fat deposition.

### Example 7

### Western Diet-Associated Gut Microbiome

To further investigate the linkage between diet-induced obesity and the Mollicute bloom, we performed capillary sequencing of seven cecal samples obtained from seven mice: (i) three samples were from animals fed the Western diet (one that had been conventionalized, two that were conventionally-raised), (ii) two were from conventionally-raised mice that had been switched from the Western to FAT-R diet for 4 weeks, and (iii) two were from conventionally-raised mice that had been switched to the CARB-R diet for 4 weeks (one mouse/family/diet; as noted above, the conventionally-raised mice were from two mothers who were sisters; Table 14). A total of 48 Mb of high-quality sequence data was generated (average of 7 Mb/cecal DNA sample; Table 15).

TABLE 14
Nomenclature used to designate microbiome datasets
obtained from the cecal microbiota of C57BL/6J mice

| | | | | | 16S rRNA | |
|---|---|---|---|---|---|---|
| Figure | Microbiome | Host | Host | Host | survey | ARB |
| label | label | family | state | diet | label | label |
| Western 1 | WEST1 | 1 | CONV-R | Western | Western 10 | MD22 |
| FAT-R 1 | FATR1 | 1 | CONV-R | FAT-R | FAT-R 4 | MD25 |
| CARB-R 1 | CARBR1 | 1 | CONV-R | CARB-R | CARB-R 2 | MD8 |
| Western 2 | WEST2 | 2 | CONV-R | Western | Western 9 | MD20 |
| FAT-R 2 | FATR2 | 2 | CONV-R | FAT-R | FAT-R 5 | MD27 |
| CARB-R 2 | CARBR2 | 2 | CONV-R | CARB-R | CARB-R 4 | MD21 |
| Western 3 | WEST3 | — | CONV-D | Western | Western 3 | WD8 |

TABLE 15
Microbiome sequencing statistics

| | Average read | Forward | |
|---|---|---|---|
| Microbiome | length | reads^{a} | Sequence (Mb) |
| Western 1 | 668 | 9,072 | 6.1 |
| FAT-R 1 | 586 | 10,681 | 6.3 |
| CARB-R 1 | 603 | 10,773 | 6.5 |
| Western 2 | 633 | 10,997 | 7.0 |
| FAT-R 2 | 723 | 10,893 | 7.9 |
| CARB-R 2 | 591 | 10,244 | 6.1 |
| Western 3 | 734 | 11,705 | 8.6 |
| TOTAL | — | 74,365 | 48 |

^{a}trimmed according to quality and vector sequence

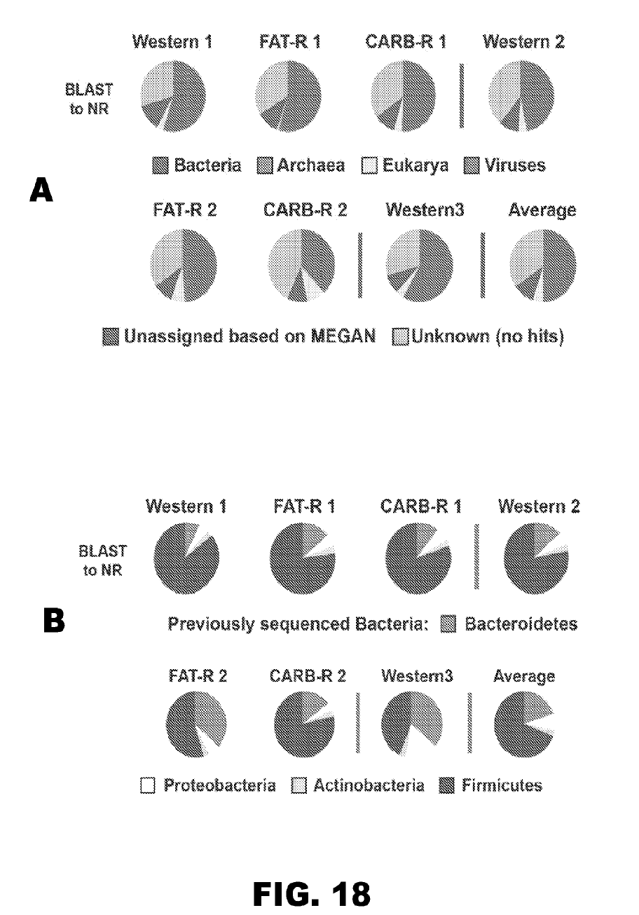

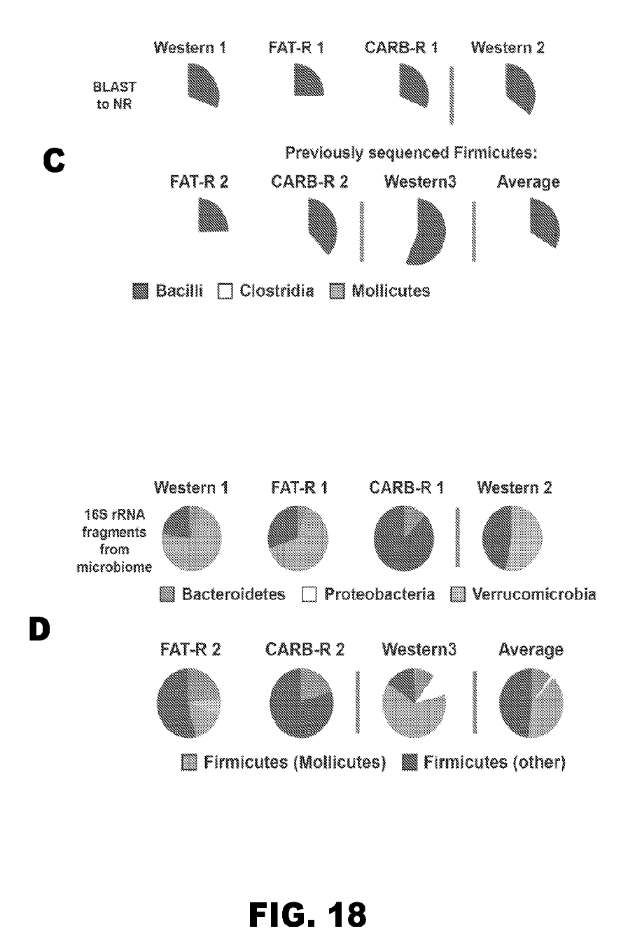

Taxonomic assignments—All seven datasets were dominated by sequences homologous to known bacterial genomes (49.97±2.52%), followed by sequences with no significant homology to any entries in the non-redundant (NR) database (34.82±1.89%) or that could not be confidently assigned (10.28±0.45%), followed by sequences homologous to eukarya (4.56±1.02%), archaea (0.27±0.05%), and viruses (0.10±0.01%) (BLASTX assignments performed with MEGAN [18]; for further details see methods; FIG. 18A). The sequences homologous to eukarya could be assigned to two principal groups: metazoa (largely derived from host cells) and apicomplexa.

Consistent with the PCR-based 16S rRNA data, the largest group of sequences in all seven cecal microbiomes was homologous to the Firmicutes division of Bacteria. Analysis of 16S rRNA gene fragments culled from the metagenomic datasets confirmed the presence of the Mollicute bloom in the Western diet-associated cecal microbiome (FIG. 18D). However, all of the datasets, including those from mice on the Western diet, had a low relative abundance of sequences homologous to previously sequenced Mollicute genomes (FIG. 18C). These results support the conclusion that the genetic make-up of the DIO-associated Mollicute bloom is distinct from that of previously sequenced Mollicutes.

Analysis of 16S rRNA gene fragments and NR-based taxonomic assignments confirmed that both the FAT-R and the CARB-R diets resulted in an increased relative abundance of sequences homologous to the Bacteroidetes (FIG. 18B,D). To focus on the microbiomes' bacterial and archaeal gene content, all sequences that could be confidently assigned to eukarya were removed before conducting the analyses described below.

Functional predictions—Metagenomic sequencing reads were subsequently assigned to orthologous groups from the STRING-extended COG database [19] and the Kyoto Encyclopedia for Genes and Genomes (KEGG) [20]. KEGG pathway-based metabolic reconstructions of cecal microbiomes harvested from mice fed the Western, CARB-R, or FAT-R diets revealed a variety of differences associated with the various diets (Table 16). Notably, the Western diet microbiome is significantly enriched for KEGG pathways involved in the import and fermentation of simple sugars and host glycans, including ‘fructose and mannose metabolism’ and ‘phosphotransferase system’ (p<0.05 based on bootstrap analysis of pathways in the Western diet-versus CARB-R microbiomes).

TABLE 16
KEGG pathways significantly enriched or depleted in the Western diet
microbiome*

| | KEGG pathway |
|---|---|
| Enriched | Phosphotransferase system (PTS) |
| | Fructose and mannose metabolism |
| | Glycolysis/Gluconeogenesis |
| | Glutamate metabolism |
| | Carbon fixation |
| | Unclassified (non-enzyme) |
| | Pyrimidine metabolism |
| | Protein export |
| | Phenylalanine, tyrosine and tryptophan |
| | biosynthesis |
| | Oxidative phosphorylation |
| Depleted | ABC transporters |
| | Bacterial chemotaxis |
| | Bacterial motility proteins |
| | Flagellar assembly |
| | Protein kinases |
| | Two-component system |
| | Pentose and glucuronate interconversions |
| | Other amino acid metabolism |
| | Starch and sucrose metabolism |
| | Ribosome |

*Based on bootstrap analysis of pathway relative abundance in the Western versus CARB-R microbiome (p < 0.05)

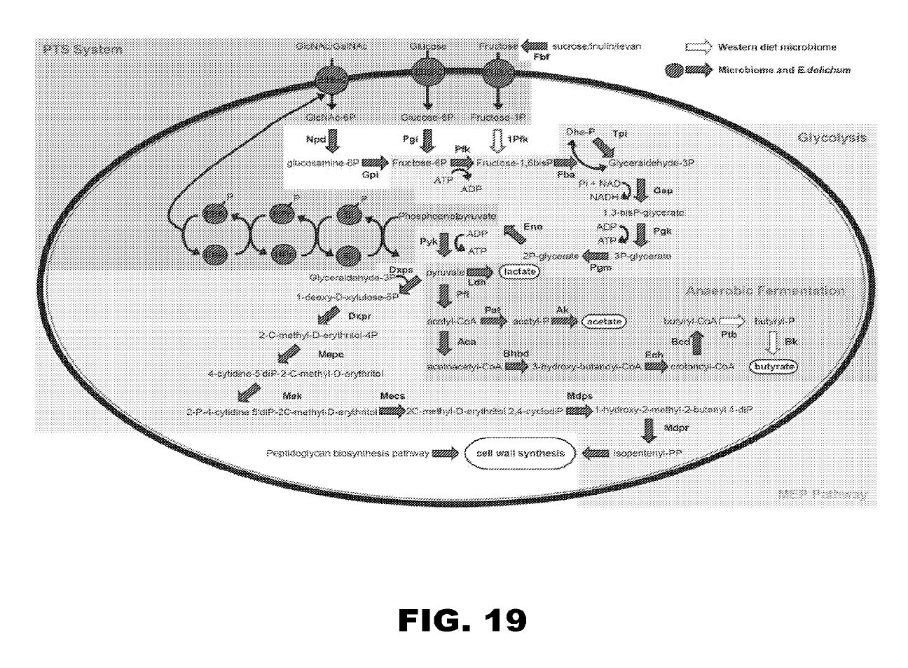

Phosphotransferase systems (PTS) are a class of transport systems involved in the uptake and phosphorylation of a variety of carbohydrates [21]. Each transporter involves three linked enzymes that act as phosphoryl group recipients and donors: two are cytoplasmic enzymes that act on all imported PTS carbohydrates (HPr and EI); the other is a carbohydrate-specific complex (EII) comprising one or two hydrophobic integral membrane domains (EIIC/D) and two hydrophilic domains (EIIA/B) [21]. Phosphoenolpyruvate, produced though glycolysis, can be used to generate ATP (via pyruvate kinase), or used to drive the import of additional sugars through transfer of a phosphoryl group to EI of the PTS (FIG. 19). PTS genes are found in multiple divisions of bacteria, including Proteobacteria such as E. coli, as well as multiple sequenced Firmicutes (e.g., the Mollicutes Mycoplasma genitalium, M. pneumoniae, M. pulmonis, M. penetrans, M. gallisepticum, M. mycoides, M. mobile, M. hyopneumoniae, M. synoviae, and M. capricolum; KEGG version 40) [20]. The PTS also plays a role in regulating microbial gene expression through catabolite repression, allowing the cell to preferentially import simple sugars over other carbohydrates [21].

Multiple components of the PTS are present in the Western diet microbiome (EI and HPr plus EII), which could allow the import of simple sugars (e.g., glucose and fructose that together comprise sucrose, an abundant component of the Western diet), as well as sugars associated with the host gut mucosa (N-acetyl-galactosamine) (FIG. 19). The Western diet microbiome also contains genes that support metabolism of these phosphorylated sugars to various end-products of anaerobic fermentation (e.g. lactate and the short-chain fatty acids butyrate and acetate; FIG. 4). In addition, the Western diet microbiome is enriched for genes encoding beta-fructosidase, a glycoside hydrolase capable of fermenting beta-fructosidases such as sucrose, inulin, or levan (p<0.05 based on a X² test of Western versus CARB-R microbiome).

The Western diet-associated cecal microbiome contains genes for cell wall biosynthesis and cell division: (i) orthologous groups COG0707, COG0766, and COG0768-COG0773 (together, found at a slightly higher relative abundance in the Western versus CARB-R microbiome; p=0.3 based on a X² test); (ii) multiple components of the KEGG pathway for peptidoglycan biosynthesis; and (iii) all enzymes in the 2-methyl-D-erythritol 4-phosphate (MEP) pathway that converts pyruvate to isopentyl-pyrophosphate (IPP; FIG. 19). IPP provides, among other things, a precursor for peptidoglycan biosynthesis [with the aid of genes for farnesyl diphosphate synthase (K00795) and undecaprenyl diphosphate synthetase (K00806) that were also identified in the microbiome]. Together, these findings indicate that unlike other Mollicutes (e.g., the mycoplasmas), members of the bloom have the capacity to construct a cell wall.

Additionally, unlike the more diverse Firmicutes-enriched ob/ob and CARB-R microbiomes, the Western diet-associated microbiome is depleted for genes assigned to KEGG pathways involved in motility, including (i) ‘bacterial chemotaxis’, (ii) ‘bacterial motility proteins’, and (iii) ‘flagellar assembly’ (Table 16). This observation suggests that the Mollicute bloom is either non-motile or utilizes a mechanism for gliding motility, such as that found recently in other Mollicutes, that is independent of the known pathways for bacterial chemotaxis and flagellar biosynthesis [22-23].

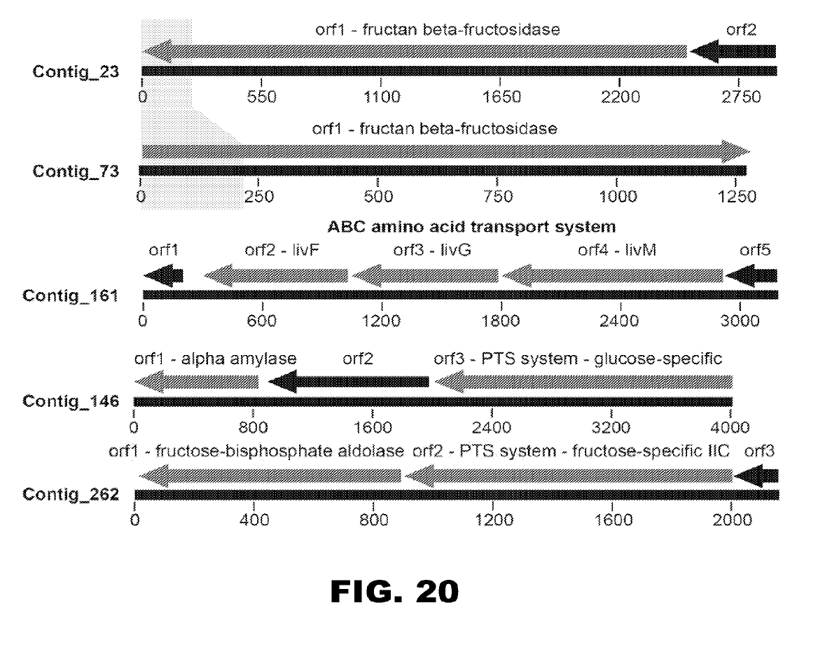

Assembly and analysis of contigs—All seven microbiome datasets were assembled individually and as one pooled dataset using the program ARACHNE [24]. As expected, the reduced diversity of the Western diet microbiome produced the largest contiguous ‘genome fragments’ (Table 17). Manual inspection of genome fragments from the combined assembly (N50 contig length=1738 bases; FIG. 20), revealed multiple contigs containing genes that were enriched in the Western diet microbiome, including those involved in the degradation of beta-fructosides such as sucrose, inulin, and levan (fructan beta-fructosidase) and the import of simple sugars (PTS genes for fructose and glucose transport). A large contig was also found that contained multiple genes involved in the import of amino acids (ABC transporters) (FIG. 20). Interestingly, the two genome fragments containing PTS genes were each flanked by another gene involved in carbohydrate metabolism: in one case, an alpha-amylase (starch degradation) and in the other fragment, fructose-bisphosphate aldolase (glycolysis). These genome fragments are likely derived from the expanded uncultured Mollicute clade: they are composed of reads from microbiomes with a high relative abundance of the bloom and share the highest degree of homology with Bacillus and Mollicute genomes (Table 18).

TABLE 17
Microbiome assembly statistics

| | | Mean | | | | |
|---|---|---|---|---|---|---|
| | | trimmed | Fraction | Number | N50 | Max |
| | Input | read | assembled | of | contig | contig |
| Sample | reads | length | (%) | contigs | length | length |
| Western 1 | 11136 | 612 | 0.9 | 17 | 1306 | 2159 |
| FAT-R 1 | 12288 | 582 | 0.2 | 6 | 1218 | 1458 |
| CARB-R 1 | 12288 | 590 | 0.0 | 2 | 1246 | 1246 |
| Western 2 | 11904 | 573 | 2.6 | 37 | 1782 | 7376 |
| FAT-R 2 | 11904 | 622 | 0.8 | 23 | 1428 | 3451 |
| CARB-R 2 | 11520 | 575 | 0.1 | 4 | 1071 | 1236 |
| Western 3 | 13440 | 627 | 6.7 | 107 | 1884 | 11022 |
| All | 84480 | 598 | 3.9 | 387 | 1738 | 11990 |
| microbiomes | | | | | | |

TABLE 18
Read placements in contigs and BLAST results

| | Western | Western | Western | FAT- | FAT- | CARB- | CARB- | | |
|---|---|---|---|---|---|---|---|---|---|
| | 1 | 2 | 3 | R 1 | R 2 | R 1 | R 2 | BBH^{a} | e-value |
| contig 23 | 5 | 5 | 9 | 4 | 1 | 0 | 0 | S. mutans | 0 |
| contig 73 | 1 | 0 | 2 | 0 | 0 | 0 | 0 | S. mutans | 4E−91 |
| contig 146 | 2 | 4 | 4 | 3 | 1 | 0 | 0 | E. faecalis | 0 |
| contig 161 | 2 | 1 | 4 | 3 | 3 | 0 | 0 | E. dolichum | 3E−97 |
| contig 262 | 1 | 5 | 0 | 3 | 4 | 0 | 0 | L. monocytogenes | 1E−119 |

Validation of PTS expression—We constructed a cDNA library from mRNA enriched total community RNA that had been isolated from the cecum of an obese mouse fed the Western diet (see Materials and Methods from example 7 for details regarding the mRNA enrichment procedure). Sequence analysis of the inserts in this library confirmed that a gene encoding EII of the fructose, mannose, and N-acetylgalactosamine specific PTS transporter (COG3716) was expressed. The low representation of mRNA-derived sequences in our library precluded further (costeffective) characterization of the DIO cecal microbiome's transcriptome. However, sequencing of 16S rRNA-derived inserts in the library provided further support of the high abundance of the Mollicute bloom: 80.6% of expressed 16S rRNAs had a best-BLAST-hit to Mollicute gene sequences (BLASTN comparisons with the NCBI nucleotide database, e-value <10⁻²⁵).

Biochemical validation of enhanced fermentation in the DIO microbiota—To verify our in silico predictions concerning metabolic activities that are enriched in the Western-diet associated gut microbiome, we performed gas-chromatography-mass spectrometric and microanalytic biochemical assays of the concentrations of short chain fatty acids and lactate in aliquots of the same cecal samples that had been used for 16S rRNA surveys and metagenomic sequencing of community DNA (See Methods).

Biochemical analysis—Short-chain fatty acids (SCFAs) were measured in cecal samples obtained from mice fed Western, FAT-R, or CARB-R diets (n=3-5 mice/group; two aliquots per mouse). The procedure, described in an earlier publication [49], involved double diethyl ether extraction of deproteinized cecal contents spiked with isotope-labeled internal SOFA standards (Isotec: [²H₃]- and [2-¹³C]acetate, [²H₅]propionate, and [¹³C₄]butyrate), derivatization of SCFAs with N-tert-butyldimethylsilyl-N-methyltrifluoracetamide (MTSTFA), and GC-MS analysis of the resulting TBDMS-derivatives using a gas chromatograph (Model 6890; Hewlett-Packard) interfaced to a mass spectrometer detector (Model 5973; Agilent Technologies).

Lactate levels were quantified using a microanalytic approach: cecal samples were quick frozen in liquid nitrogen, stored at −80° C., and lyophilized at −35° C. 1-5 mg of dried cecal material was homogenized in 0.4 ml 0.2 M NaOH at 1° C. Alkali extracts were prepared by heating an 80 μl aliquot for 20 min at 80° C. and adding 80 μl of 0.25 M HCl and 100 mM Tris base. Acid extracts were prepared by adding 20 μl 0.7 M HCl to a separate 60 μl aliquot, heating for 20 min at 80° C., and neutralizing with 40 μl of 100 mM Tris base. The Bradford method was used to determine the protein content of the alkali extracts (BioRad). Cecal lactate levels were determined using a combination of pyridine nucleotide coupled enzymatic reactions with the Lowry oil well technique and enzymatic cycling amplification [50]. A 0.2 μl aliquot (25-100 ng protein) from the acid extracts was added to 2 μl of reagent containing 50 mM 2-Amino-2-methanol-1-proponal buffer pH 9.9, 2 mM glutamate pH 9.9, 0.2 mM NAD+, 50 ug/ml beef heart lactate dehydrogenase (Sigma; specific activity 500 units/mg protein) and 50 μg/ml pig heart glutamate pyruvate transaminase (Roche; spec. act. 80 units/mg protein). Following a 30 min incubation at 24° C. the reaction was terminated with the addition of 1 μl 0.15M NaOH and heated 20 min at 80° C. Once the samples cooled to 24° C., a 1 μl aliquot was transferred to 0.1 ml NAD cycling reagent and amplified 5000 fold. Lactate standards, 5 to 10 μM, were carried throughout all steps.

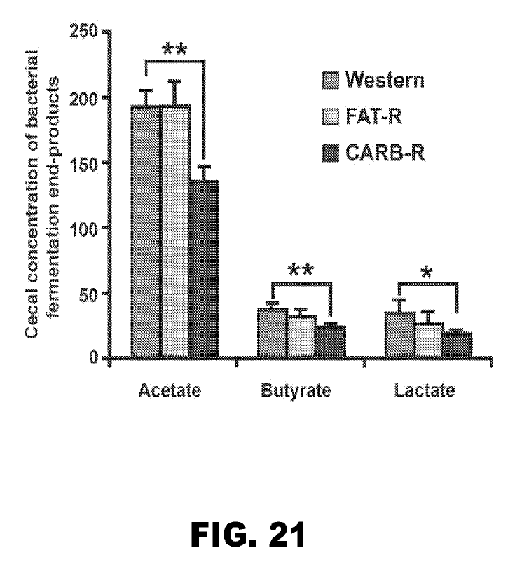

As predicted from our metabolic reconstructions, the cecal contents of mice fed the Western diet (on average, 50% Mollicutes) had a significantly higher concentration of multiple end-products of bacterial fermentation, including lactate, acetate, and butyrate compared to the cecal contents of CARB-R mice (on average, 6% Mollicutes) (FIG. 21).

### Example 8

### Whole Genome Sequencing and Analysis of a Human Gut-Associated Mollicute

Representatives of the Mollicute Glade that blooms in the distal gut microbiota of mice fed a Western diet have yet to be successfully cultured. Therefore, to obtain additional insights about genomic and metabolic features that may allow this lineage to bloom in the cecal habitat of mice fed a Western diet, and to validate our comparative metagenomic predictions, the genome of Eubacterium dolichum strain ATCC29143, a related Mollicute (FIG. 14) isolated from the human gut microbiota (Table S8) was sequenced.

Whole genome sequencing and annotation—A draft assembly of the Eubacterium dolichum strain ATCC29143 genome was generated from ABI 3730x1 paired-end reads of inserts in whole genome shotgun plasmid libraries (35,683 reads; average read length of 569 nucleotides, representing ˜9× coverage), as well as from reads produced from one run of the 454 FLX pyrosequencer (425,423 reads with mean length of 250 nucleotides, representing ˜49× coverage).

The Newbler de novo shotgun sequence assembler was used to assemble 454 FLX sequences based on flowgram signal space. This process includes overlap generation, contig layout, and consensus generation. The resulting contigs were then broken into linked sequences to generate pseudo paired-end reads, and aligned with 3730x1 reads using PCAP [45]. To minimize potential assembly/contamination errors in the draft genomes, only contigs greater than 2 kb were used. Genes were predicted using MetaGene [25]. Each predicted gene sequence was translated, and the resulting protein sequence assigned InterPro numbers using InterProScan (version 4.3) [31]. Each gene was annotated based on the output of InterProScan and BLASTP comparisons versus the KEGG database (version 40) [20] and the STRING database (version 7) [19], in addition to experimentally validated metabolic pathway maps in the MetaCyc database (http://metacyc.org) [46].

For KEGG pathway analysis, the relative abundance each pathway was calculated for each genome (number of genes assigned to a given pathway divided by the total number of pathway assignments). The relative abundance was then converted into a z-score based on the mean and standard deviation of the given pathway across all microbiomes. KEGG pathways were clustered using Cluster3.0 [47]. Single linkage hiearchical clustering via Euclidean distance was performed, and the results visualized (Treeview Java applet) [48].

A deep draft assembly of its genome was produced, based on 49-fold coverage with reads from a 454 FLX pyrosequencer (106 Mb), and 9-fold coverage with reads from a traditional ABI 3730x1 capillary sequencer (Gen Bank accession ABAWO0000000; http://genome.wustl.edu/pub/organism/).

TABLE 19
E. dolichum draft genome sequencing statistics

| | |
|---|---|
| Total contig number | 51 |
| Total contig bases | 2209242 |
| Average contig length | 43318 |
| Maximum contig length | 453733 |
| N50 contig length | 291535 |
| N50 contig number | 3 |
| Major contig (>2000 bp) number | 17 |
| Major contig bases | 2181491 |
| GC content | 38 |

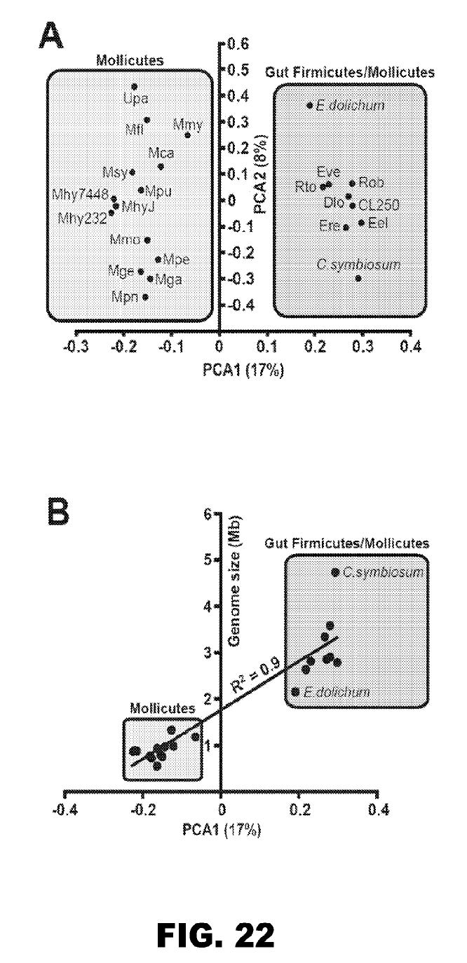

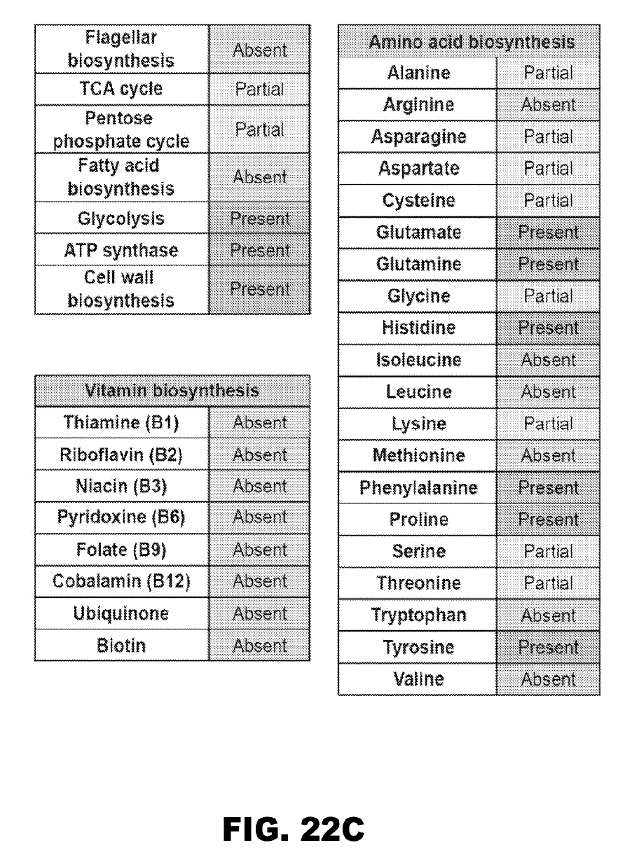

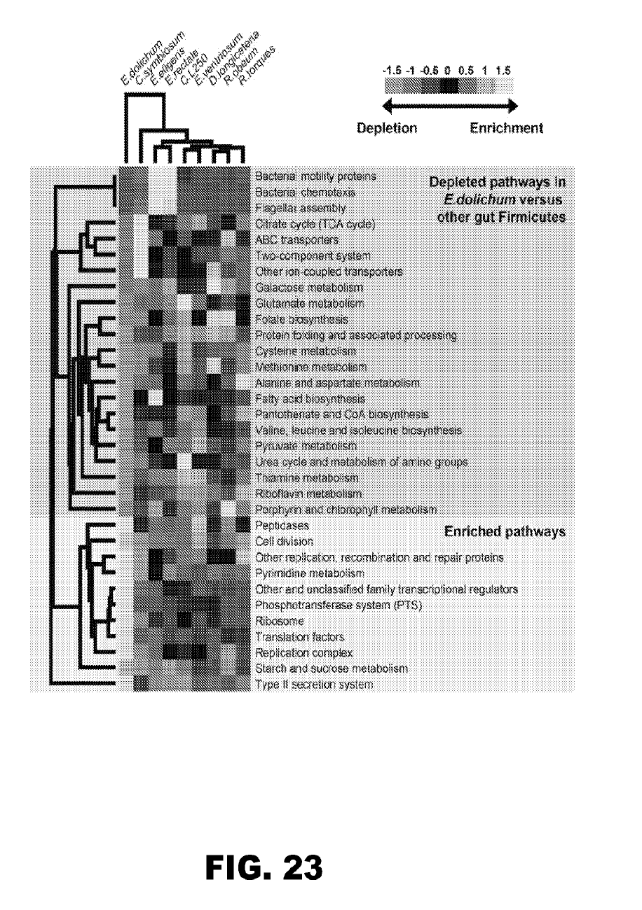

We first compared this deep draft assembly of the E. dolichum genome to eight other deep-draft assemblies of human gut-associated Firmicutes and to fourteen finished Mollicute genomes (FIGS. 22 and 23). The program MetaGene [25] was used to predict the protein products of these diverse Firmicute/Mollicute genomes and the proteins assigned to the STRING-extended COG database [19] and the KEGG database [20] using BLASTP homology searches (e-value <10⁻⁵).

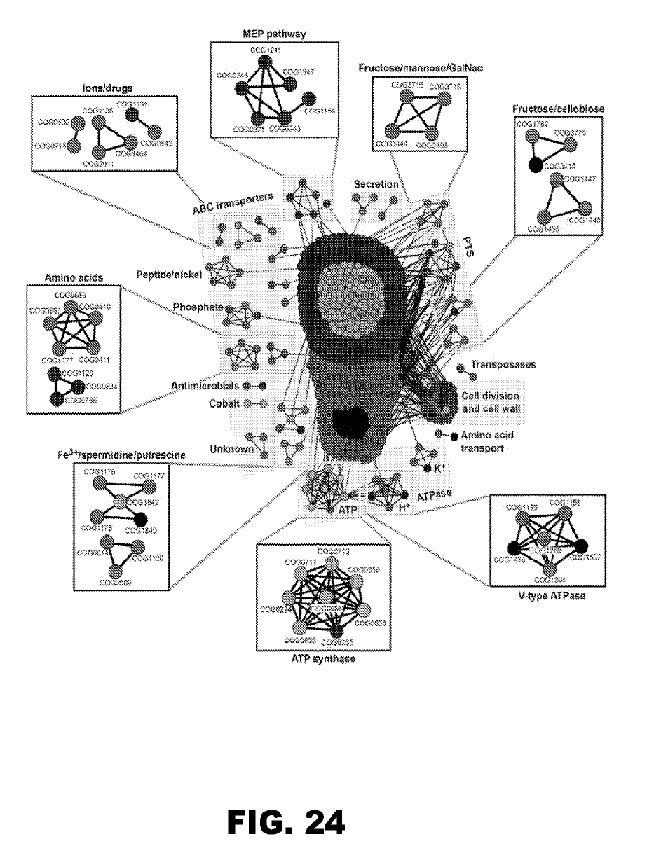

Principal component analysis (PCA) of KEGG pathway representation in all 23 genomes revealed a clear clustering of the previously sequenced Mollicute genomes and the recently sequenced commensal gut Firmicutes, including E. dolichum (FIG. 22A). The total size of the E. dolichum assembly is over twice the average Mollicute genome (2.2 versus 0.91 Mb), and two-thirds the average size of the recently sequenced gut Firmicute genomes (3.2 Mb). Our analyses revealed that the genome size reduction and corresponding gene loss that has occurred during Mollicute evolution has produced small genomes that are largely restricted to encoding components of metabolic pathways essential for life (FIG. 24). Accordingly, bacterial genome size significantly correlates with the clustering results (FIG. 18B; R²=0.9, p<0.05). As expected from its relatively restricted genome size, E. dolichum is enriched for many KEGG pathways involved in essential cellular functions such as “Cell division”, “Replication, Recombination, and Repair”, “Ribosome”, and others (FIG. 23) but is missing a number of metabolic pathways similar to other ‘streamlined’ genomes (e.g. the mycoplasma, and oceanic α-proteobacteria) [22,26]. Its genome lacks predicted proteins involved in bacterial chemotaxis and flagellar biosynthesis, the tricarboxylic acid cycle, the pentose phosphate cycle, and fatty acid biosynthesis (FIG. 22C). It is also significantly depleted for ABC transporters relative to the other gut Firmicutes (FIG. 23), and a variety of metabolic pathways for the de novo synthesis of vitamins and amino acids are incomplete or undetectable (FIG. 22C).

E. dolichum has a number of genomic features that could promote fitness in the cecal nutrient metabolic milieu created by the host's consumption of the Western diet. As in the metagenomic dataset generated from the Western diet-associated cecal microbiome, its genome is enriched for predicted PTS proteins involved in the import of simple sugars including glucose, fructose, and N-acetyl-galactosamine (FIGS. 19 and 23). STRING-based protein networks constructed from the E. dolichum genome revealed that many of these PTS orthologous groups are found in the Western diet microbiome, but not in all nine recently sequenced gut Firmicutes (FIG. 24). In addition, the E. dolichum genome encodes a beta-fructosidase capable of degrading fructose-containing carbohydrates such as sucrose, genes for the metabolism of PTS-imported sugars to lactate, butyrate, and acetate, plus a complete 2-methyl-D-erythritol 4-phosphate pathway for isoprenoid biosynthesis—all genetic features of the Western-diet-associated cecal microbiome (FIGS. 19 and 24).

### REFERENCES

1. Backhed F, Ding H, Wang T, Hooper L V, Koh G Y, et al. (2004) The gut microbiota as an environmental factor that regulates fat storage. Proc Natl Acad Sci USA 101: 15718-15723.
2. Turnbaugh P J, Ley R E, Mahowald M A, Magrini V, Mardis E R, et al. (2006) An obesity-associated gut microbiome with increased capacity for energy harvest.
Nature, 444: 1027-1031.
3. Sonnenburg J L, Xu J, Leip D D, Chen C H, Westover B P, et al. (2005) Glycan foraging in vivo by an intestine-adapted bacterial symbiont. Science 307: 1955-1959.
4. Backhed F, Manchester J K, Semenkovich C F, Gordon J I (2007) Mechanisms underlying the resistance to diet-induced obesity in germ-free mice. Proc Natl Acad Sci USA 104: 979-984.
5. Dumas M E, Barton R H, Toye A, Cloarec O, Blancher C, et al. (2006) Metabolic profiling reveals a contribution of gut microbiota to fatty liver phenotype in insulin-resistant mice. Proc Natl Acad Sci USA 103: 12511-12516.
6. Martin F J, Dumas M, Wang Y, Legido-Quigley C, Yap I, et al. (2007) A top-down systems biology view of microbiome-mammalian metabolic interactions in a mouse model. Mol Syst Biol 3: 112.
7. Eckburg P B, Bik E M, Bernstein C N, Purdom E, Dethlefsen L, et al. (2005) Diversity of the human intestinal microbial flora. Science 308: 1635-1638.
8. Ley R E, Backhed F, Turnbaugh P, Lozupone C A, Knight R D, et al. (2005) Obesity alters gut microbial ecology. Proc Natl Acad Sci USA 102: 11070-11075.
9. Ley R E, Turnbaugh P J, Klein S, Gordon J I (2006b) Human gut microbes associated with obesity. Nature 444: 1022-1023.
10. Frank D N, Amand A L, Feldman R A, Boedeker E C, Harpaz N, et al. (2007) Molecular-phylogenetic characterization of microbial community imbalances in human inflammatory bowel diseases. Proc Natl Acad Sci USA 104: 13780-13785.
11. Montague C T, Farooqi I S, Whitehead J P, Soos M A, Rau H, et al. (1997) Congenital leptin deficiency is associated with severe early-onset obesity in humans. Nature 387: 903-908.
12. Lozupone C, Hamady M, Knight R (2006) UniFrac-an online tool for comparing microbial community diversity in a phylogenetic context. BMC Bioinformatics 7:371.
13. Schloss P D, Handelsman J (2005) Introducing DOTUR, a computer program for defining operational taxonomic units and estimating species richness. Appl Environ Microbiol 71: 1501-1506.
14. Suzuki K, Meek B, Doi Y, Muramatsu M, Chiba T, et al. (2004) Aberrant expansion of segmented filamentous bacteria in IgA-deficient gut. Proc Natl Acad Sci USA 101: 1981-1986.
15. Ley R E, Peterson D A, Gordon J I (2006a) Ecological and evolutionary forces shaping microbial diversity in the human intestine. Cell 124: 837-848.
16. Lupp C, Robertson M L, Wickham M E, Sekirov I, Champion O L, et al. (2007) Host mediated inflammation disrupts the intestinal microbiota and promotes the overgrowth of Enterobacteriaceae. Cell Host Microbe 2: 119-129.
17. Peterson D A, McNulty N P, Guruge J L, Gordon J I (2007) IgA response to symbiotic bacteria as a mediator of gut homeostasis. Cell Host Microbe 2: 328-339.
18. Huson D H, Auch A F, Qi J, Schuster S C (2007) MEGAN analysis of metagenomic data. Genome Res 17: 377-386.
19. von Mering C, Jensen L J, Kuhn M, Chaffron S, Doerks T, et al. (2007) STRING 7—recent developments in the integration and prediction of protein interactions. Nucleic Acids Res 35: D358-362.
20. Kanehisa M, Goto S, Kawashima S, Okuno Y, Hattori M (2004) The KEGG resource for deciphering the genome. Nucleic Acids Res 32: D277-280.
21. Deutscher J, Francke C, Postma P W (2006) How phosphotransferase system-related protein phosphorylation regulates carbohydrate metabolism in bacteria. Microbiol Mol Biol Rev 70: 939-1031.
22. Jaffe J D, Strange-Thomann N, Smith C, DeCaprio D, Fisher S, et al. (2004) The complete genome and proteome of Mycoplasma mobile. Genome Res 14: 1447-1461.
23. Hasselbring B M, Krause D C (2007) Cytoskeletal protein P41 is required to anchor the terminal organelle of the wall-less prokaryote Mycoplasma pneumoniae. Mol Microbiol 63: 44-53.
24. Batzoglou S, Jaffe D B, Stanley K, Butler J, Gnerre S, et al. (2002) ARACHNE: A whole-genome shotgun assembler. Genome Res 12: 177-189.
25. Noguchi H, Park J, Takagi T (2006) MetaGene: prokaryotic gene finding from environmental genome shotgun sequences. Nucleic Acids Res 34: 5623-5630.
26. Giovannoni S J, Tripp H J, Givan S, Podar M, Vergin K L, et al. (2005) Genome streamlining in a cosmopolitan oceanic bacterium. Science 309: 1242-1245.
27. Duncan S H, Belenguer A, Holtrop G, Johnstone A M, Flint H J, et al. (2007) Reduced dietary intake of carbohydrates by obese subjects results in decreased concentrations of butyrate and butyrate-producing bacteria in feces. Appl Env Microbiol 73: 1073-1078.
28. Hooper L V, Mills J C, Roth K A, Stappenbeck T S, Wong M H, et al. (2002) Combining gnotobiotic mouse models with functional genomics to define the impact of the microflora on host physiology. In Methods in Microbiology, Molecular Cellular Microbiology, eds. Sansonetti, P. and Zychlinsky, A. London: Academic Press, Vol. 31, pp. 559-589.
29. Bernal-Mizrachi C, Weng S, Li B, Nolte, L A, Feng C, et al. (2002) Respiratory uncoupling lowers blood pressure through a leptin-dependent mechanism in genetically obese mice. Arterioscler Thromb Vasc Biol 22: 961-968.
30. Mavromatis K, Ivanova N, Barry K, Shapiro H, Goltsman E, et al. (2007) Use of simulated data sets to evaluate the fidelity of metagenomic processing methods. Nat Methods 4: 495-500.
31. Mulder N J, Apweiler R, Attwood T K, Bairoch A, Bateman A, et al. (2005) InterPro, progress and status in 2005. Nucleic Acids Res 33: D201-205.
32. Rodriguez-Brito B, Rohwer F, Edwards R A (2006) An application of statistics to comparative metagenomics. BMC Bioinformatics 7: 162.
33. Judd C M, McClelland GH (1989) Data analysis: a model-comparison approach. San Diego: Harcourt Brace Jovanovich.
34. Cole J R, Chai B, Farris R J, Wang Q, Kulam S A, et al. (2005) The Ribosomal Database Project (RDP-II): sequences and tools for high-throughput rRNA analysis. Nucleic Acids Res 33: D294-296.
35. Posada D, Crandall K A (1998) Modeltest: testing the model of DNA substitution. Bioinformatics 14: 817-818.
36. Swofford D L (2003) PAUP*. Phylogenetic Analysis Using Parsimony (*and Other Methods). Version 4. Sinauer Associates, Sunderland, Mass.
37. Moore W E C, Johnson J L, Holdeman L V (1976) Emendation of Bacteroidaceae and Butyrivibrio and descriptions of Desulfomonas gen. nov. and ten new species in the genera Desulfomonas, Butyrivibrio, Eubacterium, Clostridium, and Ruminococcus. Int J Syst Bacteriol 26: 238-252.
38. Hooper S D, Bork P (2005) Medusa: a simple tool for interaction graph analysis. Bioinformatics 21: 4432-4433.
39. Papineau D, Walker J J, Mojzsis S J, Pace N R (2005) Composition and structure of microbial communities from stromatolites of Hamelin Pool in Shark Bay, Western Australia. Appl Environ Microbiol 71: 4822-4832.
40. Huber T, Faulkner G, Hugenholtz P (2004) Bellerophon: a program to detect chimeric sequences in multiple sequence alignments. Bioinformatics 20: 2317-2319.
41. DeSantis T Z, Hugenholtz P, Keller K, Brodie E L, Larsen N, et al. (2006) NAST: a multiple sequence alignment server for comparative analysis of 16S rRNA genes. Nucleic Acids Res 34: W394-399.
42. Ludwig W, Strunk O, Westram R, Richter L, Meier H, et al. (2004) ARB: a software environment for sequence data. Nucleic Acids Res 32: 1363-1371.
43. Page R D (1996) TreeView: an application to display phylogenetic trees on personal computers. Comput Appl Biosci 12: 357-358.
44. Frias-Lopez J, Shi Y, Tyson G W, Coleman M L, Schuster S C, et al. (2007) Measuring microbial community gene expression in ocean surface waters. Proc Natl Acad Sci USA, in submission.
45. Huang X, Wang J, Aluru S, Yang S P, Hillier L (2003) PCAP: a whole-genome assembly program. Genome Res 13: 2164-2170.
46. Caspi R, Foerster H, Fulcher C A, Hopkinson R, Ingraham J, et al. (2006) MetaCyc: A multiorganism database of metabolic pathways and enzymes. Nucleic Acids Res 34: D511-D516.
47. de Hoon M J, Imoto S, Nolan J, Miyano S (2004) Open source clustering software. Bioinformatics 20: 1453-1454.
48. Saldanha, A J (2004) Java Treeview—extensible visualization of microarray data. Bioinformatics 20: 3246-3248.
49. Samuel B S, Gordon J I (2006) A humanized gnotobiotic mouse model of host-archaelbacterial mutualism. Proc Natl Acad Sci USA 103: 10011-10016.
50. Passonneau J V, Lowry O H (1993) Enzymatic Analysis: A Practical Guide. Totawa, N J.: Humana Press.
51. Sonnenburg J L, Chen C T, Gordon J I (2006) Genomic and metabolic studies of the impact of probiotics on a model gut symbiont and host. PLoS Biol 4: e413.

1. A method for decreasing body fat or for promoting weight loss in a subject, the method comprising altering the microbiota population in the subject's gastrointestinal tract by increasing the relative abundance of Bacteroidetes.

2. The method of claim 1, further comprising decreasing the relative abundance of Firmicutes.

3. The method of claim 2, wherein the relative abundance of Bacteroidetes is increased by about 1% to about 100% and the relative abundance of Firmicutes is decreased by about 1% to about 100%.

4. The method of claim 2, wherein the relative abundance of Bacteroidetes is increased by about 40% to about 60% and the relative abundance of Firmicutes is decreased by about 40% to about 60%.

5. The method of claim 1, wherein the relative abundance of Bacteroidetes is increased by administering a probiotic comprising Bacteroidetes to the subject.

6. The method of claim 2, wherein the relative abundance of Firmicutes is decreased by administering an antibiotic to the subject.

7. The method of claim 6, wherein the antibiotic has efficacy against Firmicutes but not against Bacteroidetes.

8. The method of claim 1, wherein the subject is selected from the group consisting of a human, a companion animal, a zoo animal, and a farm animal.

9. The method of claim 2, wherein the subject is a human diagnosed as obese or having an obesity related disorder; the relative abundance of Bacteroidetes is increased by about 40% to about 60% by administering a probiotic comprising Bacteroidetes to the human; and the relative abundance of Firmicutes is decreased by about 40% to about 60% by administering an antibiotic to the human.

10. The method of claim 9, further comprising placing the human on a calorie restricted diet.

11. The method of claim 10, wherein the diet is a reduced carbohydrate diet or a reduced fat diet.

12. The method of claim 9, wherein the obesity related disorder is selected from the group consisting of metabolic syndrome, type II diabetes, hypertension, cardiovascular disease, and nonalcoholic fatty liver disease.

13-15. (canceled)

16. A method for selecting a compound for treating obesity or an obesity-related disorder in a host, the method comprising:
a. providing a microbiome profile from the host;
b. providing a plurality of reference microbiome profiles, each associated with a compound, wherein the host profile and each reference profile has a plurality of values, each value representing the abundance of a microbiome biomolecule; and
c. selecting the reference profile most similar to the host microbiome profile, to thereby select a compound for treating obesity or an obesity-related disorder in the host.

17. The method of claim 23, wherein the host microbiome profile is identified using an array.

18. A method to determine whether a compound has efficacy for treatment of obesity or an obesity-related disorder in a host, the method comprising:
a. comparing a plurality of biomolecules of the host's microbiome before and after administration of a drug for the treatment of obesity,
b. such that if the abundance of biomolecules associated with obesity decreased after treatment, the compound is efficacious in treating obesity in a host.

19. A method of predicting risk for obesity or an obesity-related disorder in a host, the method comprising:
a. providing a microbiome profile from said host;
b. providing a plurality of reference microbiome profiles, wherein the host profile and each reference profile has a plurality of values, each value representing the abundance of a microbiome biomolecule; and
c. selecting the reference profile most similar to the host microbiome profile, such that if the host's microbiome is most similar to a reference obese microbiome, the host is at risk for obesity or an obesity-related disorder.

20. The method of claim 26, wherein the plurality of biomolecules of the host's microbiome is identified using an array.

21. A computer-readable medium comprising a plurality of digitally-encoded profiles wherein each profile of the plurality has a plurality of values, each value representing the abundance of a biomolecule in an obese host microbiome.

22. The computer readable medium of claim 28, wherein each profile of the plurality of digitally-encoded expression profiles is associated with a compound for treating obesity or an obesity-related disorder.

23. A kit for evaluating a drug, the kit comprising
a. an array comprising a substrate, the substrate having disposed thereon at least one biomolecule that is modulated in an obese host microbiome compared to a lean host microbiome, and
b. a computer-readable medium having a plurality of digitally-encoded profiles wherein each profile of the plurality has a plurality of values, each value representing the abundance of biomolecule in a host microbiome detected by the array.

24-26. (canceled)
